SEQUENCE LISTING (66 sequences)

SEQ ID NO: 1 - protein, 189 aa, Mus musculus
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 2 - protein, 207 aa, Homo sapien
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 173 aa, Mus musculus
MEHSGILASLILIAVLPQGSPFKIQVTEYEDKVFVTCNTSVMHLDGTVEGWFAKNKTLNLGKGVLDPRGIYLCNGTEQLAKVVSSVQVHYRMCQNCVELDSGTMAGVIFIDLIATLLLALGVYCFAGHETGRPSGAAEVQALLKNEQLYQPLRDREDTQYSRLGGNWPRNKKS

SEQ ID NO: 4 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 5 - protein, 182 aa, Mus musculus
MEQRKGLAGLFLVISLLQGTVAQTNKAKNLVQVDGSRGDGSVLLTCGLTDKTIKWLKDGSIISPLNATKNTWNLGNNAKDPRGTYQCQGAKETSNPLQVYYRMCENCIELNIGTISGFIFAEVISIFFLALGVYLIAGQDGVRQSRASDKQTLLQNEQLYQPLKDREYDQYSHLQGNQLRKK

SEQ ID NO: 6 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 7 - DNA, 4554 bp, Artificial Sequence
  5i - homology arm
accataacccaacactagagccagcttttcaataaaataagacagtgtgaaacaagtttaatattactcacacccaaagatatttgaatccctactgtggtgattcgaatatgctcaggcccatgggaagtgtcactgttaggaggtgtggcattgttagagtgggcgtggccttgatggagaaagtgcctgactgtgggggatgggctttaatgtcctatgctcaagctccacctggggcagaagacagaccctcctcctggctgcctgcagaagacagtctcctgttatttattgcctttggaccaagatgtacaactctaggctcttccagcaccatgtctgcctggatgctgccatgcttcacacgatggtgataatggacttgaacctctgaaactgtaaaccagagccccagtgaaatgttttcttttataagagttaccttggggggctggtgagatggctcagcaggtaagagcacccgactgctcttccaaaggtccggagttcaaatcccagcaaccacatggtggctcacaaccatccgtaacgagatctgactccctcttctggagtgtctgaagacagctacagtgtacttacatataataaataataaatctttaaaaaaaaaaaaaagagttaccttggtcatggtgtctcttcacagtaataaaaccctaactaagacaccctctaaactgaatccagtgaccagaagtttccgagataaaaaaaaaaaaattctgagcactgtccatggaagcagccatggtaaacagcggcagaggttggcatctggaggtgacagggacaacaaaaacaaaaccaataaacacagaaagtacaagagacagagacagtaaaaataaaaccaaaaaacacagaaacagaaagctcacacactcaatggagacaagtcagaaacaaaacatgtgctggctgccatcattcatacattcaattaaacctggtccatttccatcttggttcccatgcttgcaacaaaacttctgcaaagacaagacacaaaaccagaccaagaaactcaggttggcaggtgcaacatctcagttttttgccttggatgggagaatatttgggtcctacagaatgcctcctcaccccagatgggatcccaacagaaccagatgtcgcagaaaattcgttttcaggggaggcctccatagatgtttgctgcttttctccacatggtagttcactctactagtccaggaaactgaaagtacacttcgaagccttggaacttatcaaagttgacacctcctgaggtggcccatcagccccagagtcccagagtcagaggcaggctttagaatttggatgagtggtctgatcttgctggggcctcctgaaaacattaatcctatcaagtgagaataagatcactaccacaaaatctgagccaaatctgaagcaagctttattaaatacttaataaagccaggttgatgggttgtggtcaggtccatacccagtattctcagactatggtcccaagttacattagtcaggggcttataaaagcaaaatccacacaaggctacatccttcctacctttgtccaatcaggagcaagcatacatcctgacataactgctacctatagttttcacatctacacgtgattaagcatctcctgtgcagttggggtaaccatcttgtttaccaaagcaaaatttcatggcttattatcttacgtgaacaacaagcccccagcacttcaggaagttatctgtccttgaacaattgggacttacaggttagcacagtaatttaaatttaaaacataacattagccttcacatgacccccatatgtgcactatgaagtgtgtgatcgtgtgcccatgtacacacacttttaaaatatgaaaaaaaggggggaacaggatctggaaagataaggtcaggagtcacgagtactcgctgctctgtcagagtatttgaatcctattccagcgtccatgtgaggcgcccataactgcctataactcgctccatagaatctgaaaccttcttctgcctccttgggcacctacacacatacaactcacacacacaaagatacaaataaaaaataaaccttttgacagatggtggtggtgcacacctttaatcacagcattcaggaggcagaggcaggtggactgctgagttcaagaccatcctagtctacacagtgaattccaggatagccagggatatagaaagaaactctgtctcaaaaaacacacacttgatggctcacagccatccttttgagatctgatgtcctcttctggggtgtctgaagacagctacagtgagcgagtgggaagaaaaagtgtctgaaggcagctgcagtgtacataatgtattataaatataataaataaataagtaaatatataataagtaatataataaataaatctttttaaaaaacaaaaaaattaaattaaataataagtctttttttaatggaaaaaatagagaagtaactctcccgaatgaaatatgaataccacacccttgaacgttgatctgggttgccctgttcccctaaggaaactgtgtgtagggaggtgtgctcacgtacggctttaagtccagagcaggacatcaagtgtctccctctctcatttaccagctaattgccttaagacactattgggatccacctgcctctgcttttcaaatgggtgctggggatttgaactcgggctctcatgcttgcacacaaagtactgttatccaccaagcccctggcagtcattattattcctttgtagtcataaaacaaacccctaaatgcatgtattcaccaaatgcattggtgggaacagcaggtaggcaaattacaacaaagaatctccagatttcagatccactctggtgactttcttagcgttgggattggcgggagcttctggaccgtggatggcccaagataacggtggctctagttctgcaacattccaactctccccaaccacagagctctaatcggtcttcagattcttccacacagatgcagcttcttcaaggaatgtgttcattcacaaagatagcaggctgtagattcagtgggattcagaagacgggaaggttttaggagagcatgcgagtctaaagatctgaagaggagcccctcccatcttcagctgaaaactaagcatcaacttgaaaggatagggagaaagctttgcagctatcagggctggaaatagttgttccaactccacaatacaaggcactggaaagagctggaggttataaggcagaagagataaccactcatttagaaacctctttggtaaaggcactaatcccatttgggagggttctgtcctcaagatctagcctctcacagtggatctgcctgctaataccatcaacatatgaattaggagaaggcaaagataaaataaagctgggtgtgacagtacattccagtaatctcggtacttgggagggaggggaaggaagatagccaagagttcaagaccagcttagaccttgtagcaagttccaaaccagccaggactatatagtgaaagcttgtctcaaaaataaataaatagataatgagagaggcaaaatgatgcaaatccatgacagtacgacaacttgaaggtcaatacatttcacagcctagattaaactaatacattccttgaataacatgataaagaaaatatttcagggcttctcacatttttttttctactcaagacacatgcctttttgcctaggtaaattttacacaaccctacatataaaggtatacaaatcaactatttagcgatgataaatcattacatttctattttaaaacaatcttggggaccagcaggatggctcagcttatgaaggcgtacactgccaattctgaagatagagtcccccaaaacccacatggcggaaggaaagaatcaactcttgcaagctgtcctctaacctccacatgtgtgcgtggcacctgcaaatattttttttaattacaaaaattaaaacaactatttggggtgtgtgtgtgtgtgtgtgtgtgtgtgtgtgtgtgtgtgtgttagcatttattacaagtaaaagatgaggggctggagacctgggtcagtgattaagagcacttactgctcctacacaggaactgagtttggttcccagcatcctcttcaagaaactcacaatcacctataactccagctctggggcaatctgacgtcttctcctggcctctgacggcattgcattcacatgcacaaacacacacacatacataatttaaaataaagataaatctttatttttagcatggaagatgaatatgcttatttatttctgaataaagaactaaatcttgactaaatattttattcaaagggcatatagcaccatcagtgtttataagagttgatcgatatttttggcttttagggctgaaaatgtactcggttggtagagtgcttgcctagtatacatgaggtttgatccacaccattgcacaagccaggaatggtggtacgtgcttgtaatcctaacactgtga

SEQ ID NO: 8 - DNA, 5274 bp, Artificial Sequence
  3i - homology arm
tctatccaaatagacatttctttttttaatgtttgctgggttcttacgcaggtcataattcagtgggccagtgcagcagtgaggtggcacatcaagaaaaagtatctgccacacaaatgtgacaacctgagcccaatcccctgagaagcagaaaaagagaacccactccaccatgttgttctcttacctccacatacacttgaagaatatgcacaccccatgcatacgcatcacacacacactatagtaataacaataagtaaaagcataacaatgataataataacaacaactactactactcagtgaatcacatatgcagtgaaaggtctccttccaccggagtgtccagccacagagtagccttccccacaggcaagtgagcttcttacatatgttcttaggtgtatactaagcatataagaaaagttctcaagtacactttattctcctttcccctatgtctcacactatgatataacactatatatcttgaattttctcctaatccaacctgaagattatgaaggataaagtagacaagaccaattactggctaaggattgattgattgtagcattttacttaaagcacagtttgtgctgcctgtttgccaggcatgacttcacttgctacctggaacaaaatgatgtggagacatagctgaaacaacaacgcatgtgtgaagaactccctttggagggctgaagcgatggctcagtgcttaagagcactggctgctcttccaagagacctgggttcaatccccagcacctgtaaaagtaacttttattcatgtgatgtgattgtcgtctctaagccttactgcctctgtctgctatcctaggcctagtcctggaagcttccagcctctgtacaacatggtggagtgggttctctttttctgtttctcaggggattgggctcaggttgtcacatttgtgtggcagatactttttcttgatgtgccacctcactgctgcactggcccactgaattatgacctgcgtaagaacccagcaaacattaaaacttagaatgttttgtttgttttggtttttttgagacagggtttctctgtgtagccctggctgtcctggcactcactttgtagaccaggctggcctggaactcagaaatctgcctgcctctgcctctgcctctgcctcccaagtgctgggattaaaggcgtgcgccaccacgcagggctcacaggtttcattcttgacgttttattttatggttttggtgtttgccagcatgtatgtgtatcacatgtatgcctggtgctttatgaggccagaaggagttagatctcgaaaaaccaaaaaaaaaaaaaaaaaaaaccaaaaaaaaaaaaaaaccaaaaaaaaaaaaaaaccaaaaaaaaaaaagaagcctgtatgatatataattagcctatgataatatttgccaattttaagtgatttcacctgttctcagcagtttttagtaatttgccccatcataattcaggttgaaaacatcaccaccactgttttaatagtccttatgctgactctcaaccccaagcagccacaagtctctgtctcttcaagttttctttttctttattgggagagcttgattgcagaggcgaaaccccaagcccagcactggtgctgcttatataggcctaggagaggcgtgtctcacacccggattggttatgcattctacctcatttgcatgcctcacacctgattggttatactctcagtacctcacagaacctcattatcatgcccgggccaggcagtgtcttggcaaaaaacgttattgcatatgtacacattggttgtttacccaaccgtatgcgtggtggccagtggtagccagcgtcaccttgtaatggcatatgtggcttcccacaggtcctgctatctatctagccttgaactccaagtatatgtgtattttgtttcttcttgctgaaaagccatttcatgggcagtccactgcttgtccattccccagtcaaaggacacatgatggtttcacttctcggctattataagcactgctgcaatgaggattcttttgaaatgttctattttgtttttattttggttttttcaagactcagtttctctgtgtagccctagctgtcctggaactctctctgtatatgaggctggccccaaactcacagagatccatctgccctgtctccaagggctggaattaaaggcttacaccaccacagcccagcgtgcaaggctctttatgcaactataatttcatttctctggagtaaagacctaggaatgaagatcatatggcaaattcacttttagctcttaaagaaacagccaaattcttttctaaactaacagcatcgttttacatccctactgacatgacagctttttgtttttatgaatcttgaattttcaacaatatttgtcactacttcaatatagccacattaacatttattttgtattatttttattttgtgtatatgggtgttagggctgttggtctatctttgcacaatgggcatgcagagccaatggaggccagaagagggcattgtatccctgagagtggagttagtgattctagctgctaggtgcatgctgaaatctgaaccctgcctcctttggaaagtaaggcggtgctcttaaccgctgagtcatctcttcagctcagagcaatggctttttacacaacacataatattaatctttgccccaacctcactttcttaatggtgacttttgaaatgcaaaagtctttttgataaattccattttatagattttgttgcatttgggaggtcatgtctaagacaagattgtctcctgtctcattatttgtaggtcattgttagggatttggtctagctttaacaataaatgtcacttagaatatatgtaaagtattagtaacaaataagaataaaagatatcattgaaaaatatctgaacatgtaattttattaaactaaatctcacttcatagcagttatatagatatacactatcaagtacagcaaagaagtgcctggtactcctatgcatgtatgctaccaatctcttgctgaggtattgagctgtgccaatataattttttttaaggggtatctcagaatagggtagggttttggttggttggatggttggtttgaagtaaccctaggttagtccaaactcactgaatagcagaggatgacttcggatgctcaattctcctgtctctatgtaccaagtgctagaattacagcatgccccacggtgtcccactcactacagttttaattttcattttccttactaaaactaagaaccaaggctggagaaatggctcagacgacccagattcaattcctagcacccacattgcagctcacaactgtctgtaactccatatccagggaatctgacaccctcacacagacatacatacaggcaaaacaccaatcaatgctcataaatatttttcaaaattaaaaactaaaaattaaccagacttgtaggctgtatactgttagcattgtatagataaatctaagaccaacccaaaatagcgcgctgcctggcacctaatacctgctcaaaacgtcttatacattgtccttgttggtattgttactgcttctaagccacaacgagtgaaacggctccaaaaagccataggacagaagggagcgtaaaaagcaaaaagtcacagtctgtgcagagcccgacaccacaatagtgtatctgccctcaagagatggatttccaaggtctagcttcatattccacaaaacttcaaagcacgcagtcatagccaaccctgccccgccccgccctgctgcctagcccaatcccaaagccacttccggcccgccccccaccctcgctagctgccagcaaacgtgcgcatgcgcctcctcggtgttttctctgtcattagcgaagcccgacaccgccctacttccggcgcggggtaaagacccaacaggctcggcgctcagcgatgatgtcagctccggttgccttgctgagtcggaagtaagaaccttttggtcgctgtagctgtgaggtgtcgtcgtccctgccctttcaggctccggtaagaggcaaggtataagaggacgagagtgttgaggctgcgacccggttttctcctaagcaattgccagtcaaggcccctgcatgcctgctgttctcaactgcttgcctgacccgggtccgtcttgaacttcgaccctgtctaatctggaaacccttacggtgctccggcgtccggtggctctgcacattcccttcttctttgccaagtccctgctaggaaaggcccgagtgtgggctgtggctgtcttttgacttattttatctaggacctgcgcagggtgggtgggtctccctgctgcctatgaatgttactgaaccccaaaggataaaatacgaagagtgaacgacagtgccagatgccaaggagacaaaatgaagctcgactcatagcaatcaaaccagtagggtgcgtgtagcaagaggttgacttttaaagcgagtgacggcttcccagctgtagctctttggattctgctcgagtttttcttggtcgagaaaacatttctcagattgaccatagagtcagacgttttgtgtttagtattgttccttttaggattcttttttgtctcctagagcaagaatgggttaagtctcaatgtcattgacttttttggtcccagagacataaaatggggcccaagatgaaatgccagggtttgttagagacggttttgtttgttgtgttgtgttctttttttgttttcggacgtgtatgtgcttcacttgatgagatcctagcttgcagataaacgtattgcaaggatttttgttacgacacctaatgtaacactgaaggtatttccaggtagacatgttactttgggaacatttttaaattccaaaatagagactttatggagtacaatttaatacagtgtaatttgggcttggggtagggagttgctggggttataaactaagccctggtgcatactaagtgctctagaacagagctacacccaaccccaccttagccatagaacttggctatagcaatctgtttttcagtgttaccaatagttctactttaaaactggattggacatgatcttaaaatatctgtatgctctcaggagccagtagtggccttaaaggattagcagcactctatataagtagaagcttgagtcaggagaaaatgagagaattgtatgggtccaatggttgacacttcctagagatgtag

SEQ ID NO: 9 - DNA, 17070 bp, Homo sapiens
gtggctcatgcctctaatcccagcacttttggaggctgagtgagtggatcatttgaggtcaagagttcgagaccagcctggccaacatgatgaaaccctgtctctactaaaaatacaaaaattagctgaacttggtgacacatgcttgcaatcccagctactcaggaggctgaggcaggagaatcacttgactccaggagacagaggttgcagtaagccaagatcatgccacagcactccagcctgagcaacagagcaagactccatctcaaaaaaaaaaaaaatccctgtctcaaactcctgctttccaggtagatggagcgggataatgtgctttaattggtgaagggtttctgatccccatttatcatgcagaaggcaggagcaagccagagacaggtcctggcgaaaggaagctaaaatggtgcctttgatacaggggaaagggatacgaaaggggcttaggtgagacctccttacagctagtggcacctggtcagcagggccgtagagttgaggccatccccttagcagcagtgggactttgttacacacaccctctgagtcttggtgagaaactagtcccagggccccttgttgtctctgccactcccattcatgacatcatggaaacttctgtgcggctgggctcctgaacacagcagcctgccctcggctggatgaggcagccaaaaccctaaaagggttgtgatagggactggagtcaacaagtagctgtgggaacgtggtggcgcagacccttcacccagggagccctgtgggctagaaacaccagacagccaggatgtggcgagagactgcccaacacactgtctgtgtgaggcagaggaaggaaggagatgaaggaagaaacaggtaggtaggagggcgagatcccaagcaagtcaggacaactcccacgccttgcccaggagccttaagaagcccaggcacctgctgagtgaaagaggatatatttattggctgagcaagaagggaaggtacagttggtaatggctgcttctagaagccaccagtctcaggttcacttgttccgagcccagtttcctccaaggtggctgtactgagcatcatctcgatctcggaggggctaagagaggagaagagaaaacggtcaggaggcagggttagaactcttcaaggaagggccccagcaggccctgacgatgagagctcctgcctgacctctccagtcacacccagcaatgaactccccagtaggacccttcccacgtgcaaacagcattcagtgtgagcctctctatccccacttaccctcccttcattcctgcctccttcccctcaacgctcacctgatagacctggtcattcctcaacagagcttgtgtgtcggcagctagaagaaccagagagagacatcaatggcctagcagatgggactgtgagatccaccctcccacaccctcagaagtctgcatgagtgatatgaacacacaagttctttcaacagtcaaagttctaggagtctttaggagattgcaagagtaactcccagctgagactaaacctaccaccagccccattccttagctggtgcataagctcactggtacacacacacacacacacacacacacacacacaaacacactctcatgctctgctcttccactaacccccagacagccttccagtctcatgtccagcaaagcagaagactcccaaagcaaggagcagagtggcaatgacatcagtgacaatgatgccagccacggtggctggatccagctccacacagctctggcacactgtgggggaagggaggagagaggagaggttgagagcctttaagatcagggaaccatcctctgcctcctagggcaacccttagatctcttatgccaaaacccaaacttcaataagaccctgggagaaaggcctggtgatgggcttgccacaccttcctccaccctgcatcctaaggaatccctgggaagagccaagaagtaatctcagcttttgggaccttgaacaaggtggtgggccaaacctctcacccaaccaaggctgcagtaacatgacccctactgctctgtcttgctgagttaagctctctcttccactttgaaggtccccaaatctggcttgtaccattacaatagtgacctcactttgtttagagaacagatgggttcaccatatccctctctagccagaaagttctcacatccagaagccctatccattccaacccaaagggttcaggaagcacgtacttcgataatgaacttgcacggtagattctttgtccttgtatatatctgtcccattacacctatatattcctcgtgggtccaggatgcgttttcccaggtccagtcttgtaatgtctgagagcagtgttcccaccgttccctctacccatgtgatgctggtattgcaattcacaaacactctgtcctcaagttcctctataggtatcttgaaggggctcactaaaggggaaaaaatatcacagttggagacagctctttgatctgcaccaagccctttgttctgcggaagctcatacttaacagagaccattttcctggtccaggacagtttatggcttccatcaagagagacagaagtcacaagaaaaagccttcagaaagttccccaccaactgcaggggtcaagggggacatgaggatgccattcaagcagaggacaggtcttggggccttggtgcaaaagaggacccctcagagcaggattgacccaagcaccttcctggaaatgaatccagaccactgatgaggagtagggggagcacggaccactgaagcacctggaagatgtggaaagacagaagaacattcctcgattggaaatgtctgcattttttcttcaaggaaacatctaattccacttcccagccatctacaacactcccacttcagcttcttatcctcatccttcctcattgcccctgctccattgacaaccaagaaagcggggctctcaacactgaagcctttcccagggccagggatggctgtgggtggagaccagctggtttaccagcccctgaattatcagccaagtggtccagaacgggaccagggcaaatcccatgtacagttttccacccttggttagaaggaggagaacaggaaaaaaattttattgaatccatccctagagctcctcacaagtcaagtcttgtgggagacttttagggctggaggtgagtgcagcaacattccagatgcagtgagttcctctgacagcctgagcacatctccacaggccacagaggcactacagtctatgcctccaaacacagggaaaagtggaggctacattcattcatcctgggcttcacactaagtcccaaatttggatacaagagcatcttctagaaaaccctgaaacagctgttgctcacacttctgaagcaggttggaagtatatgcatgtatcctcagggagacacatgcacatcaaatgcttcacgtcctacagtcgcgtcctcttcagggatctgtctccagtggaaatcctgagtgccctagtgcagccaactattaggtgaccattggacccagtttgcttagtgttgaaggggttcctcggacatgggactttccattttaaaactgaaattggcaaactgagatgagttaaaatcctaccatgtaacaacccctcaaatcttccctccgtcctgctcaacctaaagttaacttctcttaaagcattcacataagtgctaggacatgcctccagggatgacataatcatggccaaacaaacaagagtcctgattccagaggccatcaggcctaaaaggagtagtgcaggaagctgtgctcccatggccagtcccagattcaggtacatacgtactgagctattttctgcagatctctggcctaaggccttctgagagacattctaggcccacatgcacccatggctggagtcagtcaaagccaagagcctgtttcccagactctatgctacatcctgcccctgccctcctgacacccctggggtgcctggtgaactgaagctagcaccgagaagcacttttttttttttttgagataaggtctcacaggttgcctaggttggagggcagtggcatgatcacagctcactgaagccttgaaatcccgggctcaagtgaccctcctgcctcagcctctcaaatagctgggactacagttgtgcaccaccatgcctggctaattcttttgttttttgtaaagatagagtctcatcatgttgctcagactggtctcaaactcctggcctcaaaggatcctcccacttcggcctcccaaagctctgggattactggtgtgagccaccgtgcctggcaagaaacactttcaagtgggcctcactcccatcagtaatgtccctctcaggtcccttcccccacccacctggagtagccttaccttgcgagagaagggtagccagtaccaggccagagagaaacgtgctatgttccatctcccagcggaactcatccagtagataaagccaggtcaccgaactatcagcctgggtgagagctgccctcccctagctgactcacaggtaccggaaagaggagagtgggggaggaactagaagatgtctgcttctctgctttctgccgttgatggtgggaaattttcagagtgggggtgggctaggggtagggtaggaaggaagcggtgtaaattgctctatctctgagcagggagctggcagagaatatggaaaaggtggcctgaactttagcgtccctattgacaatgcaaccacatttacacacaacctaaacactgccacatctcgaagccccttgagagaagccgtcggccccatagcgcaagccgtagcagctagatttctcatggaggctgatctttctcaggacccttcactaggcagccagggacaccagatctagcagcttcttgtcagtgggaggttgggctttagagaccccagccagagatttgaatcctgggtccaatactgcctacctgtggggcctgggccagccataaaatttttcagagtcttattccattagtaccattattaggattcaaacaagatatttgcatggtgcctcacgcatcatatgtgctcattaaggggtagttattaataataatataattgactgacaggcaatattgagcctcccggtgagacaaatggacctttttcccctgtggcctacgaggatctgaaactcttcacgctgctgcagttagactgtcacttacctggggacagagtcatgcctgtcttgctcactgctgtatcttgtgcctggcacataacgggagctctgcacatttttgttggctcactgactgactggctgagggagataggggcctgagatcctggacattcagtccgggctctggcccctgaaaatgtgctggcctgtcctcggaattgttccacctattgccttccaggcgcctctttcatgatctcaaaagaatagtgaaaccaggtgccgtgtctcacgcctgtaatcccaacactttgggaggctgaggcaggtggatcacaaggtcaggagttcgagaccagcctgaccaacaaggtgaaaccccgtctctactaaaaatacaaaaattagccgggcttgctggcacgcacctgtaatctcagctactcaggaggctgaggcaggagaatcgcttgaaccccggaggtggaggttgcagtgagctgagatagcgccactgcactccagcctgggcaacaaagtgagactctgtctcaaaaaagaaaaagaaaaaaaaagtgaaaaaaattcctgaatgaaggcctggactgaggtggctttccatttggaggtccagccccaagcatctgagagtccctcctaaattcattacctacagcaacaacaactagcaacaagtaacaactggctacgatcctaacaactaatgacagggacatttatctccctcatgaagaaacggtcccaggaccatctcccacccagcatccattgcggttccctgtgcaagatgagtctctgagtgggaatccagcactctctccctcttcttccccaccaccttcaccctccttaacggaaaaacaaaaggcatctgcacctgcagccctgctgaggcccctgctgctcacacttgcagcagagggtggaggctctgggttcttgccttctctcaaaggccccagccccaacagtgatgggtggagccagtctagctgctgcacaggctggctggctggctggctgctaagggctgctccacgcttttgccggaggacagagactgacatggaacaggggaagggcctggctgtcctcatcctggctatcattcttcttcaaggtaagggcctactaggggtctggaagcctggggaagggctcaagggaagagcccatcactagtgagacaggaatattggtatccctaaccttcagcctacctctgctgtcaccttagagttcaaagaagggcaaaatggaggctcttaactgttctctgctagagagaaacagtgtcccatggaggagaaggaatccttgtctctgaaaaatgcaaacagagtacttaaatggctgaagagaggaccctgttaccgccatcttagatttgaatgcagcccaaaagggcataggccaagaaactaaaaggaaaaagtatatgttccctacttcagagctgggggctagcagtcgacctaggaaatgtccattcactcagttgggcagttggccaattggtcaacaaagatgggtgagaccctaatatgtgatatgccatgaggaaataaaaatgaataaaacacttatgctctacagaactcatagtctcattggagagattagttttcatggtgccagactgagtgtacagtatagtatgggtgcctgcatgaatactccattggactatccaataggaagtggatgaactgggttgatgcctggagtctggacatcctgggaaagttaaaaccatggaatttgctgaagtcacacaagtactatgtgaggaagagggagctgaagatggaacagtgagggcaccaattttaaggggcagttagagaaaaaaaaacattccaggaaagagataaaaggagaatcaaagagctgaaataatcaaatgtatgcactaccttggaaaccaagagagtagagagcttctagaagaagagaattaccaagagtataaaatgttaaagagagcccactaatataagaatatatttaaaagtttaagtctgcgggatgtggcagaatatgcatcatgggtgatttagcacagtttcagcagggtattgtgagtggaagccaagtgaaaattggtagaaggtgaaggtcaggtgatgaggtcagcaagtgtagactattctttaaagacatttggatgaggagggagaaaagactgggtggtaggtagaggaggaagtagagtctaagaataaatgtttttgagggtgagagaaacttgagcacatttctaggctaaaggagaagatgatgagtagagcaaggtcctggagttgatggtggaaggacagagtcaagagtggggtcaagagcacagagaggaaggcaacgttaccctctaagaccaggagaaaggaagtgcaaacggcactggccacaaataggtttataaagggaaaggaagctaaaggagaaaggacaataacaacttccgtgttctaaaaaatccaggtgggaggcaatgccacctgctgaaactgagctgggtgatgaccggttgggagacttgaagaggacagtgagagtttagaatagaattatttctgagagaaatgcaaggaaaagtcaggtaaagacaacacaggttgaggagcaactctcaagcctgggtgaggtggctgcatatcatggaaagagcatttgagttcagcagtagaaaacctgagctctggatccagcttggacaccaatgagctatgtgtacttgaacaactcctccactgtctgggagttctcatgccagccaatccagaaatgtggcagggacgggcatggtggctcatgcctgtaatcccagcactttgggaggtcgaggcaggtggatcacctgaggtcaggagttcgagaccatcctggtcaacatggtgaaactctatctctactaaaaatattttaaaaattagtggggtgtggtggtgggtgcctgtaatcccagctactcaggaggctgaggcaggagaattgcttgaacccaggagatggaggttgcagtgagccaacacggtgccactgcactccagcctgggcgacagagtgagactccgtctcaaaaaacaaaacaaaaaaaaaagaaatgtggctatatgaactcttaagtcctaaaaggaaacctcactcagatatgcaacattaaaagatgtcaatcagccaggcacagtggctcatggctataatcccaacactttgggaggctagggagggaggatcatttgagcccaggagttcaagaccaacctgggcaaaatagggataccctgtgtctacaaaaactacgaaaattagccaggcatgttggcatgcacttatgccagctacttgggaggctgaagtgggaggattgcttgagcccaggagcttgaggctgcagtgagccatgatcacaccactgcacttcagcctgagtgacagagcaagaaaaaaaaaaaaagatgtctcaaaaaaaaaaaaaaaaactatgtcaatgatcagtgcaatctccttccctgaatccaatttagaatgaggggtctctgtcttctctcggtcttctgaccaggtggtcaggagaaaaactagttagaaacctctttaactgtttgtcttgccttttctcccatttgctcatttattcatttgcacattcattcaacaaataaagttcctaccatgttccagacactgcactggatactgttcaaaaataatcccgccctgcatgaggttctaagacagttcacttcttgtatttccattatactttgactcatgaccgcctctccctatcacacacactccacctctagccacactacctctcttgtccttttccaaagctgcatcttattcagctattctttctactcagaatgactttctctatctggtatattcttgcttactcttcaaggcccagacaaatgtcgtttcctctattaagctttccctgatcctcacccttggacaaaattaatagcttctacccttttttcatatgcagtcatataagcactaaaagataatgtatacattctattaaattaattgcatatatgttttaattttttcacttcattcataaactttggaagcagcagttgattcttcatatcagcactcttttctttcataatatctaattattttaggtcataatagtcctcgttaaatgtttggattaaatctaattgaattgaattcaagtgccagatctctgtaatatgtctaccatgcaactctactaccctaaggttttgtttgtctgtttgtttgtttttatttttttgagacagctttaacacctaggctggagtgcagtggtgcaatcttggctcactgcaacctccacctcccaggctcaaccattcctcccacctcctgagtagctgggactacaggtgcataccaccatgcccggctaatttttttgtattgtctgtagagacagggtttcgccatgttgtccaggctggtgtctaactcctgggctcaggcaatctgcccacctcagcatcccaaagtgctgggattacaggtgtgagccactgctgccagtcaactctgagttatgagacagatttgaggactaattgaaaagctaacttccctgggacatccagggtttttataataaaaggatcaccaaggctgaatattttataaggaactcagcctaaaggttttggatagtgcacacccactccttgccagggcattagctgctcaagaagcagagtgttctggactggataggcaatatccttacttacatatactacaatacaattctgatgctaaccccccaaattagtgtcagaccctacaggttaagagcatggttcccaaccagattgccctcactttaaatgccagctacaagttcagggtaccccagaccactcacatttctgacaaactgcctgcaatcttaaggattcccatgaccctcctcattgataatttgctagaataactcacagaatttaagaaagtgctgtacttcctatttcagttttattaaaaaataaaataaaatcagaaccagctaaatgaaaagggcaaggtctaggagggtcccgaacacagagcctccgtgccctctccccgtggaattagaacacatcgcccccagcactctcccacactctgccagcacattgatgggttcaccaaccaggaagctccaccaagtttttattgaggtatctttacataggcatgattgattgaatcattggccgcttgactgatctcaatctctaggatcccttcctggggctgataccactagtttcaaagctgcaatcctcttaccatatggttggtcttgaccagcaccatcctgagtcattgccatgcataaactcaggtatggtctaaggatccaccacagataacaaagccactcctgtcactcatgaaattccaacggttagaaacaccctcccaggataccaggacaaagatgagacaaattgtttattatcaccttgaattcaattgagtgatttagtctacaatccggaaaactaagtatagatactaccattttcatggatttggatctttcttcatcttggcctcaaataaccatggaaatacttcagggcatctgaacaactccatgcccagctaatactctatctctcttctgtctttacaggtactttggcccagtcaatcaaaggtaggagaaatggcttctttctatactcagactcagaatattgacggaaatttggcttcctacaacagtagtcctacaggagcgaacagtttagaatgaatgaaatgacggggatagagaggtgatgtctctattgtcaaccaaatcagtgacctgacataacctgttccgggcagcttgcctgtagctaagcatttaactggtctcttacaggagaagcaggaccctagtagctagggacacatctcaaactgtgacccatgaaccagtaggtattggcgtcacctgggatcctgacgttagtctctgtcaatccttctctttagttcatctattctacccaaagtgatctcatcatctggtatgctgttagcagtttcttacctgtatagtatcttccaaataacatgccccaaaatcccaaagttttacccctactaattacagcaatgtctcttttattcttcaccccctgacgcagatattggcgtcacccgagagcatgttagtaatgcagaatctcccctccccagaactactaaatagcacctgaaattttaacaagatccccatgtgattcatgtgcacatcaaagtttgagaaacactactctaatgatctcctggtatgcagaagcagggagaatttcagaggcaagatccttaatagaaccacggcttttctcatttcaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaagtatgtaatcccctttggtctgtttgttgtgaaattaatcagtatttgctgttctggtgagctttttatctggggtgaaagtggaaatagatcctcaacagtaatattatcgcctgttctcttaatttcagcttgcctcttttaaaatactgtaagatacttccctcaccctattgaaaaactacagccagtcctgtaaaattttgtttacctttgggtgggctccatggattcaagcaattcagcactgagttgaatgaaggggttggggagacagaggtgatcataaggtgagcagcagtgagaactggagcgcagtggagcatgaagagtatctagtcttccttctgttccagatacttcctggtagttagactacatgggcttccccaggaatcctgggggacttaagagcatcaagatgcattgagttttggcgcaaggatcttcccttgccctgccacccacagaggaaaagcctgctgccctccacagcggcattattgcagacaggcaggagaaaacgaaccaggaaaaacaactttcgcaacctgaaggtttgtctctccttttccctacagtgtgtcagaactgcattgaactaaatgcagccaccatatctggctttctctttgctgaaatcgtcagcattttcgtccttgctgttggggtctacttcattgctggacaggatggagttcgccagtcgagaggtaaaagaatgctcttagatgagagatgggaccacctgagaccctcagctttcctcctaccattatgtacccaatggaagagactgagttggtgcttcttgctagtgtgcatagttgggtgaggctgtatttctctgagacaggagaaaggatacttggtgttatcacagcatgttcacctgtctcaagatacagctccctctgtgaaaaaaaaaaaaaacaaaaacaggcgcagtggctcacgcctgtaatcacaacactttgggaggccgaggcgggcagatcacgaggtcaggagatcgagaccatcctggctagcacggtgaaaccccgtctttactaaaaatacaaaaaattagccgggcgtggtggcgggtgcctgtagtcccagctactctggaggctgaggcaggagaatggcatgaacccgggaggcggagcttgcgtgagccgagatcgtgccactgcactccagcctgggcgacagagcaagactccgtctcaaaaaaaaaaaaaaaaaaaactattttaagtaaacccttacatacagaaaagtgaatcacaagcatacagctcaaaaaaaaaattcacagagcaaatacacccatgcagccagcacataatttaagaaagacaatattaccaatgccattgaagcccctcttgtggtcacttccagccactacccatcctcaagagtgagcattgtcctgacttctaacaacaaggattaactttgcttgtttttgtcctttatgtaaatggactcatatactaagtattcttgtgagtagcttctttcactcaacaacatgtttatgtgctttatccatactcttgtgtatatataaaactcttattgttatttagtattccattttgtgaatataacccaatttattatgcattctaccttgtgtggattctgtttcttttttgtgcagcttcagacaagcagactctgttgcccaatgaccagctctaccaggtaaggggatgaagaataaaagagacattgctgtaattagtgggggtaaatctttgggattgaggggcatgttatttggaagatcctatacaggtaagaaactgctaacagcataccagatgatgagatcagtttgggtagaataaatgaactaaagagaaggattgacagagactgaggtcaggatctagtgagcacaagttgaagaacacactgagagggacacacgaagaaactctcgacaggctgggcactcactatagacaggccatggctcatgcctgtaatcccagcactttgggaagccaaaacaggtgaatcacctgaggtcaggaattcgagacaagcctggccaacatgatgaaactctgtctctactaaaaatacaaaaaaaattaaccaggcgtggaggcgcgcgcctgtagtcccagctactcaggaggctgaggcaggaggaccacttgaacccaggaggtcgaggttgcagtgagctgcgattgtgccactgcactccagcctgggcaacagagaaagactccgtctcaaaaaaaaaaaagagagagagagaaaaagaaaaaagacagagcctccatctccttgtcctctttccatcctcaggaccatgaagtacccactccaaattctcacatataaaaaacattcaataaacatgcatcaaattaattaatagaggatggaaaaaatgacttatgactgtgctgtcctttccagcccctcaaggatcgagaagatgaccagtacagccaccttcaaggaaaccagttgaggaggaattgaactcaggactcagagtaggtgggttcttcaatgccaattctaataaaggacccttgcatcaactgccctcgcaattgcttctaagtctagctcccttccctaagcggctataagcatcagactctggggatcagggattgggacgtggtttggggtactcttttctaaaaattctggggccatactgattgtcttggcctaggtaaatatgaattttatgtatctgtaaatcctgtcagagcagggcctcaagccatagagatgctgaatattaatcttaacctacatttgaatttctcattatctacactattaacattttgggctaattaattatttgtgatgaggggctagcctgtgcattgtaggagttatggaagcatccctggcctctctccaccagatgctggtagattgtccagtgtgacaatcaaaaatgtgtccagacattaccaaatgtgtccaaacatcacctccagggcaaaatcacccttagttaagaaccactaacccatattaaccttccaatcaataaatcaatcagtcagaagttatgatttaattaatctatctgaagtttctatcaggaagacagggttgaaagcattatttgttttttttgaacaaattgcaatttttcttttttcagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgtaaaagaaaaatgaaaagatcaaataaccccctggatttgaatataattttttgtgttgtaatttttatttcgtttttgtataggttataattcacatggctcaaatattcagtgaaagctctccctccaccgccatcccctgctacccagtgaccctgttgccctcttcagagacaaattagtttctcttttttttttttttttttttttttttgagacagtctggctctgtcacccaggctgaaatgcagtggcaccatctcggctcactgcaacctctgcctcctgggttcaagcgattctcctgcctcagcctcccgggcagctgggattacaggcacacactaccacacctggctaatttttgtatttttagtagagacagggttttgctctgttggccaagctggtctcgaactcctgacctcaagtgatccgcccgcctcagcctcccaaagtgctgggattacaggtgtgagccaccatgcctggtcttaaaaccagtttcttatatatctctctggaggtattctaggcatatatgagcacattctcaagtacatattatcctcccttcccctatcttttagacaaatgatatcaaactatacatcttgtgagattattgcataccattatatgaagataccattatatcctttttaatgcaaccatattgtacaaatagactatgatttatttaacctgttatctatcagtggatatttaagttggtagttggttccaatcttttgctcttacaacaattctgcaatgactaacattgtataaatatcatttttaaaaataattgcattgaagcataatgtacatgccataaaatccacccatcttaagtgatttcacctgttctcagaaatttttagtaaatttaactaattgtacagccattaccataatccagctttaggacattttcttttttttcttttcttttcttttttttcttttttttttttttttgaagtggaatcttgctctgtggcccaggctggagtgcagtggcgcgatctcagctcactgcaacctccacctcctgggttcaagcgattctcttgccttggcctcccgagtagctgagactacaggcacatgccaccacgcccagctcattttttgtgtatttagtatttgtgtatctagtatttgtgtacttagtagagacagggtttcaccatgttggccaggctggtctccaattcctgacctcaggcgatccacccgccttgacctcccaaagtgctgggattacaggtgtgagccaccgcgccaggcccgtaactgtattttaatatagccattctatggatttaatatggtattttattatggccttaatttgcatttccctagatactaaccatgctgagtgtcctgtcttgtgtttattaaccattcatatatttttagtgaaatgtgtatcaaatcttttgcccatttttaagttgacttatttgtttgtcttcttactattgggttgcatatgtttttgatataagtcctttatcagatatatgatttggaaatattttctaccaatctgtggtttgtttttcttaatggtgtcttttgaagtgcaaaaggtttgaattttgaagtacattttattgattttttcttctatatattgtgcttttggtatcatgtctaataaatctttaccaaacccacagttacaaagattttctcctgtcttctttttatactttttacagctttatggttttagctctaacaataaatgtgattttgaacatacataagactatttgtaacaaacacaaataaattgaattgttgggcacttgtatttgcatttgtaattttat

SEQ ID NO: 10 - DNA, 100 bp, Artificial Sequence
  connection sequence
cacaaataaattgaattgttgggcacttgtatttgcatttgtaattttataagcttgatatcataacttcgtataatgtatgctatacgaagttataggt

SEQ ID NO: 11 - DNA, 100 bp, Artificial Sequence
  connection sequence
cttcataacttcgtataatgtatgctatacgaagttatggatccgagctctctatccaaatagacatttctttttttaatgtttgctgggttcttacgca

SEQ ID NO: 12 - DNA, 5246 bp, Artificial Sequence
  5i - homology arm
ctcagatgttcctgcaatcatgcctttgctctgccaccatggactctggctctaaaactgtgatccccaggttaagggctttcttttataaattgccttcactgtggtgttatcacagtgataggaaagtgactgaaacagacagtaaatgctgagataggacagttgtttatgccagagctgggccatagtaagaaaaacctggacccctgaaactgatgggatgacatgggatgggatggcatgagacaggataacatgggatggggtagaagggatgagaggagaaagggtaggatgagagaggaagggagaggaggggaagaatgggaagggaggggatggaataggatgagagaggagaaagaggaggagaggggaggggagggattgaatgagaagggatgggatgcgagtcttgattttccagatcgtccttgaaccattaggcctacagaattggaccacacatttaaaggcctcacatgatgtgtcctataaggcaccactaaccccattcccctcctggccatcaaacctgttactagagccaattctcaaggtgactcaactggttaagtactagagaggttaatgtgccaagtatcgcaggctcaggctcgtgctttgaaagctcagtccccagttaatgataatgctttaggtggctgtggaattgttagggggtgtggctaagctgtcagatccaggaagtcacaaggttggaggggtgcagcaataatccactgtgagatagaatgaatgaactctatgtgggagatgaatgccactgctactggagttcatacccaagactcttaagctgtggtggtttgtatatgctcagcccagggactggcactattagaaggtgtgaccctgttgagtaggtgtgtcactgtgggtacgggctttaagaccctcatcctagctgcctggaagccagtcttccactagcagccttcagatgaagaggtaggactctcagctcctttggcaccatgcctgcctggatactgacatgcccttgtcttaatgataatggactaaacctctgaacctgtaagtcagccccaattaaatgttgtccttataagagttgccttggaggctggaaagatggctcagtggttaagagtattgactgctcttccaaaggtcctgagttcaattcccagcaaccacatgatggctcacaatcatctgtaatggggtctgatgccctcttctggtgtgtctgaagacacctacagtgtactcatataaataaaataaatcttaaaaaaaaacaaaaacaaaaacaaaaaagttgccttggtcatggtatctgttcacagcagtaaaaccctaactaagacagaagtcatctatcacttcttcaccctcagatcagactgtggctggctacatagcaagctactccctaagcaacggctggaagaggagggtgaagctgagctttgttgtcaaatctcacctgagcatgtgagtgaaagtcaaatatgaaaagctgcaactgtgcagctccactcatggctgaagacagccataagaggagaccctcccactggcagggacctggcattgtacctgatcatctactttatatgggaagggaaatggcctaacgtcctcagacacaccagaagagggcatcggatcccattacagaggttgtgagccaccatgtggttcctgggaattgaactcaggacctctggaagagcagccagtgcttttaaccactgagccatctctccaggcccccacccccaccacgagacctcactcctaactaaggagctattgacagttagtgaccactaagagaggaagaacacttttctttgggggtgcctgcttgtaggttggccttgccccagtggatgaccttacactcactcacttccacactcatgcacccctgattaggatttactggactcagaggactagtaataataattggggaagacaatttaggtggataaaatctaaatacattatattcatgttcaaaattttcaaagacaaaataaaaagtttaaaagaaaacttctggagggctggagagatggctgactgctcttccagaggtcctgagttcaattcccagcaatcacaaccacctgtaatgggatctgatgccctcttctgttgtgtctgaagacagcgacggtgtactcatatacataaaataaataactaaatctttaaacaaagaaaaggaaaggaaagttctggttagagagatgcctcagaggttaagagcactcgatgctcatatagaagccccgagttcagatcctagcacccacctggcagctaacagcagtctgtaactccatcttccaggacctgatgccctgttctggcttcctcaggtactgcacacacatgatatacatacaatgcatgctggcaaagcactcgtgcgtataaaacatcttttttataaaaaggggtccatcaatatcctgaagggactgtttgaggatgagggaagcaggtgacggggcactgtcacctgaactggatctgcgtgacactgcctaggacccccagctgatagggttgctgccccctgagagtggcaaacagccagaccataggtatcaccctcgggagagcctactgcctcacccaagtccacaccacctccggggaccagcttccatccaactactgggtaacgtgccccttgccatgccataactcaagaaatcagagacagcgctttactgctctcctccctcctggcggctctcatgctcttcctgacctctcttcctcaatgtctttaaggtttttagaaggaaaaagtttaattatggaagtgatacatcattgcttctcagacgtttggctaacatcaagtgtggaagtaagaccgtgcatgtattaaaaccaatgaagagcataggagaggtgtataataacaccacgccccactcatcccaccactatgccttttctcaggggcacaaccacctcaacggaccccttcctctgggcttatcttcacatcccacaaagtgcacagacatcctttgagttccctgggaaacaaaccttgaggtggagatttgtgtgcaggtggctcgtgggaatgcaggacacctgcaaaggagagagggaagccacaaggcagacagagaaagtcaacttcagggagcgacttcagggagcggcagctcagaggcctctgcttgggcctctgtgctcatgagctcactgcagctctggttaccatgcagcatttcagcaggcagcaggtaactgcactcagagggtggggggcgggtgaggggggttggggaggggagagatgtacgggggatgtctgagggcagtgaaagggagaaatcaaggtggatatgatcaagatacattacatacatgtatggaattgtcggagaataaataaatcttttttgaaaaaaggggagagagcgccagagagtgagacagcaaggcagaaagcaaaccccaactcaaaaacaaggcggaaaggggactcagactccaaactaaggtagaaagggggatcgagaaagagcgcttagtatccatctctggcctccacaagcacctcacacgtctgcacactcaaactccatacatacatgaaaagattttgtaatttccattaattaatttatttactcattttacatcccaatctcagcccccctcctccaagccccccctccaagtccccgcccctcagatagccgctccccccacccctccttccccttcccatctgagaagagggaggtccctctgggcactgagtctgatactatcccttatctggagatgtgggtgggcaaaaagaatgtaagagtagaaactagggagaagggctgtgaagcactgccttctagtagctgtggcagccagcagtggatccacatgcaactgggcttatcaacagtcaatcatgcattggggcgggacttctggggctctaagcctttctactgaactataggttatagatggattcagaaagaaaggcagccgttttcttcagtcctgtacccactgaggagctcttcaggctccttcagtagttagttccaaatccactgtcacacagctaactctcgttaaacccagtagatatatcaagaagcggagccagagagatacctcaacagataaagttgtttgtggttaaaaacctaaagacctgaattgaaacctgggactcacatgttagagagaatccctcaagctaccctcgacttccacacatgctgtggcaagtctatgcccacacatatacacatataataaataggtaaataaataacgtaaaacttttttttcgtaaaactttttaataaaagacttgtcgagaaagggagattaatcatcagaaagtgtgttacaattgtatgaaattatcaaagagctaagtcaataagcgttttttacaaagaaaaagattgttgcttataactcaggaagtacttatgctagattttaaataataatgctaaaagctaccctagtgggtgcatgagtaaagcccctagcatagtgtcagtacaaagacaggagctatgtgactctctcattgtccagcaactgcccagcatgtgcaatcaaaggccaggacggctacatgctaaaaatcctgtctcaaagaaagaaagaaaagaaaagcaagcccctcgaagattattaatgagacggctccactccatcctgctgctcagacccaagggcaccataacagaaggcagatactgagtgcttcaacgtcttcctgcctccgctggaggggttatgcataaggaaggctccctcagtgtggaggtgctttgcacagggcacaagtaggtgtttttcacagcactctcgggggcttgctagagccccccttgttcatccttatgggaaggctgtgaggcgcggatttcctcagttaaccaaggcggaggcttctgcctcaaacatttccaagtgacgtggagcaggcgggccctgagtcccctctacacttcctgtgtggggttcagacacctgcctcccctgtctgcgtctggtgccttcttcagaaatggtaagtctctccttgtgaagtgccccccactctccttcatcctactatggcaaccaatgatccagggtctttgcatgcagccactgtgttgaaaacctcatctgtttctttttcagaagtaatgagctggctgcgtccgccatcttggtagagagagcattctgagagg

SEQ ID NO: 13 - DNA, 3547 bp, Artificial Sequence
  3i - homology arm
aagtgctgctgaacagagccagggaagacgagaaaagccgtgcgtgcgtgcgtgcgtgcatgcatgcggttccctcctgccctggaggcagctgtctgaagccccaagcagtagtggttatacagcaggatgtgggctgtgctgggagtgaggatgagagaggtgtggtggtagccacggaccagacactggaggctatcgatgagagaatgtggtccttggtctcccacatactctgcttgcaggagagaggaacccagagcccaagcaagctatcctaggccagctcaagccatcggactctcttctccacctttctgccctcagacacatccctgctgacttaagggctatttgttatgtgactttagcttctgtgcctagcagcggtccacacactgtccatactctgccttggatttgtaactacagttcctgctagttggtctccactaaactgtcaccttttcctctctaggcatcgatgcttatgttaaaatgagtcttgatgattttgcctatcaccaaacactaataagcaggtggcagattttgtcctgcatatatttgatgtgtccagtcaaggacattcgtagctcaacatgataaaaagcattaacatttggtgtttactgagaatttaccatgctccagaagttgttatagccctatatgtgctgtctcatttaatccttatagagacgccacaaatatatgttgtcatcttctatatagtgggctcagagaggtagcatcatttatccatgtgaagtgaaatgcagggctacaattagcagctagagatgtctaattctggggcacttttttttttttttttggatacagagtctcactatgtagctctggctgtcctagaactatgtatgtatgaactaagcatgtagactagaaactatgtagaccagactgtcctcaaactcacagagatcctcctgcctgtgcctctgcttcccgagtgccaggattaaaagtgtgcactagtaatgctctgcttctaactccagcactctggcacctacccactatgaccgagaccatggcaatgttcttcttcctaccatatgcctctggaaaaaatggaggagttggagatggagcagaaacctagagtctgttttctatatgggtcggaggcaggatacaataggatgaaggcagatgccgtgtcaaggaaagtgagggtcatgcaaaacacagctcttactccaaatagaatagcagttagtttattctggagccagcaagatggcttagcaggtgacagtgcttaccaccaagcctgaagacccgagtttgattcccagaacctacctggtagaagaaaaccaactttcataaattatcctctgactgctaaccccaccaggctagaagagcactaccacaatttatgagccaaatttgaagcaagctttttactggccaagtctatggatactggccagatccatacgcactatctatccccagagctccagccctttataaggcaggggctataaaggcaaagcccataagactaccgacttcttcccaccttcatctagtcagggacaagcatacaccctgccacactttctgcctgtgaacctgctggcaatgggtaagcaagcacatcctgtgcagctggggcaacccagcttctttatagaagcaaaaactcccggcttgttatcttccatgatcaacagcccccagcatcccaggaagttggctgttcttgagcaagtggggcttacaggttagaggcattttgtttaatagttagatctcttaagcacagtaacttatacttagacataatgttagcctcttgcgcgcgccggactggccagcagtaacaacgctgcaacaggatccttctgcacatgtttattgggagagcttgattgtagaggcgaagagaccccgagcccagaactggtgctgcttatataggcctaggagatgcgtgtctcacacccggattggctatgcactacgcctcatttgcatgttcctcatctgattggctactctctctctctctgtacctcacagagcctcattatcatacctcatttgcatgtctcacatctgattggttatattctcaaagcttcattatcatgcccgggccaggcagtgtctttgcaaaaaactttactgcatatgtacacattggttgtttgtccaaacttatgcgtggtggccagcagtagtcagcgccactctgcaacggcacatgtggcttcccacattagccatcatatttctttcttgagctgtgtatcctgagtcaaatccttgactctaaagtgggtagcggctcaaattaagatctaataaccatcagcttaatggcacccagacagcaatttacatatatagttaagatattaatgatgcaaggttattagtaactagcagaaacagaagggcaaaggccttgtgatggctgacagcagaactactacctaagtggagcccaaaaatgagaatgggagccagtcatttgtctcatcttggggcctgtttttctgtaggtttgggtttgctttgttttgttttgttttgttttggaggggttgtttgtttttttagtacattcagactatctcttatgatttcctgagtggtttgcaaagaagcaacaatttttttaaaccatacaataacctcattttacatagagcaggtcgagtgtcctatcatgttctatcatatttcagctaataaatctacttgttaatagatctgggtctatttggctttagcaggtacctatcctcatatgtcaataggctctataggtgcctatttgtcctatataccgtggagtttccttttaatctaaccttccagccttttgtagagggtaaagggtaatgtactctcttctttaactacttcctgtggaaattaggacatcactgacagggctcaggaaggctggaatgctagtcaaaggccaggaggggattcagcagaagcattgggttagctggcccagctgaagagacagggagcaactatatctgctggactgttttgtccacatcagcattcagcaagtccagggcttttagtcacttggagcaggttgtgtagtccacagctgattcctggatggtgtttgtcagccaagtggaaattcagctgggacaggtatagaccacggacagaagtttaccagtgtttgtaatttgtgcttaaaatccacgttgtagaaagggcagctggcaggtgtctgcaaaacatctggagtggactcatgcctttgagtcatagagaaagctttcacttaaaaagcttgaacattctttttctgtccagtggtctaggtatagtgatgcgaaatacctttaaatctacacttagaagacaaccaaaggaatttaaaaccttgagctcatggctgataatggcagtcagcgcttttgcagcttatcagaactccactgatgaaggttaaaactctgaacgtttggaaccttagcatgatgacaacatagcaagaaggaaaggcatctgaaacatttcttgt

SEQ ID NO: 14 - DNA, 10590 bp, Homo sapiens
atgcagtcgggcactcactggagagttctgggcctctgcctcttatcaggtgagtaggatggagtggaaagggtggtgtgtctccagaccgctggaaggcttacagccttacctggcactgcctagtggcaccaaggagcctcatttaccagatgtaaggaactgtttgtgctatgttagggtgagggattagagctggggactaaagaaaaagataggccacgggtgcctgggagagcgttcggggagcaggcaaagaagagcagttggggtgatcatagctattgtgagcagagaggtctcgctacctctaagtacgagctcattccaacttacccagccctccagaactaacccaaaagagactggaagagcgaagctccactccttgttttgaagagaccagatacttgcgtccaaactctgcacagggcatatatagcaattcactatctttgagaccataaaacgcctcgtaatttttagtccttttcaagtgaccaacaactttcagtttatttcatttttttgaagcaagatggattatgaattgataaataaccaagagcatttctgtatctcatatgagataaataataccaaaaaaagttgccatttattgtcagatactgtgtaaagaaaaaattatttagacgtgttaactggtttaatcctacttctgcctaggaaggaaggtgttatatcctctttttaaaattctttttaattttgactatataaactgataagtcctctctacttcacagattaagaaattgatactcaaaaaagttaaataacttgttttaaaccacatagtaagtgccgaagccaatctgtgagaccaggactgtttgtactctaaatggctgcaccacatgaggcaaaatggctcgtgatggttttatttcaaagacctagaaaacactatcacagctggtgctcccgtctcagacccacagcaacgatgtctccacttcctgcttcatcttgggtttctcacgtcttgaatgtgcacacaaatcacctggggatcttgttaaaaatgcagcttctgtttcaaaagacccagactggaaatggagattccgcatgtctagtacgctctcaagtttattaatctgctgctggtcctaggaacatattgagtagcgaggggcaggatgtgactcctgtaaggagtggccaggcattttcttagagacctgtgttataaagtatgcttttccttaaaaaaaagaagaaggaggaggaggagaagggccaggtgcagtggcttacgcctgtaagcccagcattttgggggaccaaagtgggaagatcacttgagctcaggagttcaagaccagcctgggcaaagtagtgaaaccccatttctacaaaaaattaaaaattagccaggcaaggtggcacacacctatagttccagctacttgggaggctgaggtggaaggatcacttgagcccaggaggtcgaggctgcagtaagccatgatcaagccactgcactccagcctggagtgtctcaaaatagataaataaataaataaataaataaataaataaataaataaaagagacagtatcaaagacccaatcacctctagacatctggcatcataggaatgtgcccagtctgctctggggataggaaagtggggatcctgtctccccctgtgtagaggtttcagtaaaagaaaggcctaggtgtgcagaaagctttcaggcaatgccagggaaactgatcattgtaatgaatccagggtattgctgagtgagggcatcctggagggcccggtggaaatgtggtcaggctcttcaatgcacaggccctagttgatgagtaatcagggtttcaaatatttccatctctgtctcaagcagaaaacaaatggaaaaactgaaccaccagaaaagcagagccagagatggaacaagaatcccagtgtttgtacccaaccaagagcgtgtttttcttccacagacaccaatgttcaaaatggaggcttgggggcaaaattcttttgctatgtctctagtcgtccaaaaaatggtcctaactttttctgactcctgcttgtcaaaaattgtgggctcatagttaatgctagatgcttccttcctctatttccccccaaatttcctgggaacccctggtcaataccagcagtaagttccactgttctagggtgtagaaatggctgtgacccagcagcaagagggaaggacatcagatgtcatcagtggtcatactgcaacacagccctttttctgtttaggaatgcaggtacccacaacatttactaacactttttttttcttatttattttctagttggcgtttgggggcaagatggtgagatatgctttctttctttcttttttatgaaatcaccccatcattctttgtagttatgaatggagctttctcttaggcctcccacagaacttccacagaggtcaggaaaaggagtttctgccatctacccctttgactttcctcacaagtctggagatatttctagcccagaagagggaagcaacagaggcaggaaataatgagtcttaaccatacaaaagaaaaattgagacttaaatgaagttgaaagcactaacagttttcatttgtttgcatttcatatttgatgtgagattctgcagaggagacgtagccagaatgcatgcacagggttactctggataagctgctggggcaacatttggatgtgtgttcagaatcacatgtctgaatactctgaatatatgtgtgtacatgtgtatttatgcaagtgcacatgcatatgagtgtgcccggcctgaacttactctctcaaccacagcggtagagtcaggagtgttccaacattggaagcccctctattcaatcagctcttccaaactgagtgaaccaatgttgtatttaatggcaaccatggctggacaccatggctcacacctgtaatcccagcactttgggaggccgaggtgggcagatcacttgaggccaggagttcgagaccagcctggccaacatggcgaaaccccgtctctactaaaaataccaaaatcagccagacatggtggtgtacgcctgtagtcctagctactcgagaagctgaggcaggagaatcgcttgaacctgggaggcagaggttgtagtgagccgagatcacaccactgcactctagcctgggtgacacatcgagactgtctcaaaaataaaataaagacaaccattatgccagcctagattccgccatgctgcctaatttgtagtgtccttaggagccatttttgtaaatagtcatcagataagatgtaaggcccataacagctttttctatgcagctgagggaattggaagatccattgtttcctaagagttgagggaagagtcccaacccacgggagcagggtctgatcttcattgccgatagaaacattactaatggcttcttactgtttccttttcaggtaatgaagaaatgggtaagaagatttccactctatctagcaaaagttttcaaatatggaatgaaatgctcatagagtacaatcacagtaacaaaccctgagaactaaaactattaaagggaaaatacaagtatctttcaatgggatccgtatgaaacttgcctgtatttgttgctagctgtcatgtcagattatagctgtgcatatatgtatctctgatcatacacatatggatgtgggttggagctaccatgtgtttttgtataagccatgaaatctttgaaggcagacagagacagtgtctcatttacctagcccagtgtctggcacatagtaggtgctcaatgaatattttttgaatgaataaatgaacaaacatatgaacacattgctaattacctcccctcaagaagctgatggtcttgtgtgagagacaaataattgaaaatatagtgagttgcatgttataatatgggtagatacagagtaaaatgaagtataaagaggggagtggtcaactctactgagtgtcgttgggaaaggttccctgggggaggtggtccttgagctgaattttaaaggataagcttatgttttagggaagaaaaatattttatgcagaagagataaagctgtatagtatgaggataagagtctaactgagctagatcagaatgtttgaatcttggctcaactctctacttgctgggtgtgtttgagtaatttacctaacttttctgtgccacagcatcatcatggtacaatggaaataatagtgctacctaacttgtagggttattatgaggaccaaatgagtaattcatttaaggcacttagaacattatctgacataaaaggcagtaggagggccgggcatggtggctcacacctgtaatcccagcactttgagaagccgaggtgggaggatcacctgaggtcaggagttcgagaccggcctggccaacatggtgaaactccatctctgctaaaaatacaaaaattagccaggcatggtggcaggtgcctgtaatctcagctactcaggaggctgaggtaggagaattgcttgaacctgggaggcggaggttggagtgagctgagattgtgccattgcactccagcctgggcgacagagcaagactctgtctcaaaacaaacaaacaaacaaacagacagtaggtgaattttagctattaatacatggaaagcatgctgactatagatgataagcattaaagtttactgagcatgtatgttttaggcattgctctaaatattttacttgaatttcctcatttaattcttccaacacccctactgtacagttaaggaaacaaagcctcaaataaatacagaaataaacaaaaataagtaaacaatccagtcctggggatataaatgcagatttaggccaagtgccatggttcatgcctataatcccaacactttgggaggccaaggcaggaggctcgcttgagctcagaaggttgaggctgcattgagcaaagattgtgccactgtactcgagcctctgtggcagagaaagaccctgtctctgaaaaaattaataaatagaaatttaaaaataaaaaaattttaatgcagatttatatgataccgaagttcattttctcaaccattatgaaatactgtttctggatatgtataaaatctttgtgagcacacatatctttttttaacttaactttcattttaaattcaggggtacatgtgcaggtttgttatataggtaaacttgtgccatgggggtttgttgtatagattatttcatcacccaggcattaagcctggtacctgttagttatttttcctgatcctctccctcctcccaccctccaccttctgagaggtcccagtgtgtgtcatttccctctgtgttcatgtgttctcatcatttagctcccacttctaaatgagaacatgtggtatttggttttctgtcactgtgttagtttgctaaagataatggcctccagtcccatccatgttcctgcaaaggacgtgatctcattcttttttatggctgcgtagtattccatggtgtatatgcagcacatttttttatccagtctaccactgacaggcatttaggttgattccatgtctttgctattgtgaatagtgcacaatgaacatacgtggagcacatttctgtctaagcacagacatctagacccttgtgtgagcatgagttaagtctaagctctgctactgaatttgtgccaataaaagttgtgagcaattttctttacatttttttcaaacaaacacacccagcagagtataatgtctatgtactttatttatgatttctagttcatttaacatgtctaagaaacatccgtgttgaaaaattatttataaattaaaataatataaactatctactgtccttatactcaactcccaattataagcaggtggaaaaacctggagaatgttttgtttacattctgtgcagtctttgtcagagggctgcctgagcaactgggtcagagtttagttctgctctgggagtagcaggacctcaagaaggaaaggaggaaaggaagtaactttttcttgagcacctgctatgtgtcattcactttcaccttcataatccatttaattttcgcaaaaactttgtgaggttggtgttttatctccatttccctgataaagaagttgaggttcagcaaagttaaatgacttgccctcagtcacacagactagggcagatccaggattcaaactcagggcttctgactcttgagtccagagctctgtccctgacagcagcagcactgcctctcctctcttccagctgttatgtccagactgtagcagaacccagtgttccagccacaagttttccaggaaataataaaggactcctagctccacctcccagggcaaaaatggctgctgtgggaaacacaggctggacctacgaatggcattagtggtttattagttgatttcagttgtccacactaataggcctccctctaacaaaaataattgagagctgattatgctcagatataatgtaaagtgaagccactttttattggaagaagcattccctcaaaacgtgtagagtatttcacattatttaaaggcaaatagagagaaaattatatggaataagaacaaagatgtttcttctctattatgagggactcagttctgagaaaggattttaaattgtaagaaataggtaagtccacgaatcagtgattcagtggtgtggagagctttatttctgagaaggccagtagcgctcccttctgacaagcaaatctaagacctggatgacagatgacttcctgcatttggttggttcttttgtcattcatatctatctgtaatacagttctggctaatttaagaggataagcttgaagacctctggaatttttcggctttaggactttaaggctttctgagcttcagtagatctagatctaggagctcatgctggtatattctgaatccgatgtatctgagttacatctatgagctacttaataaatatatctatgagctaaatctcataggctaagcatgaacctcacctccaagactcggggttcctaaatggatgagaccctctttgggaagtcttgtgggcagtgtctaattccactagaaaagttttacctacaatttaaacttaaaccatgatattttcttactgctgtttccttttttcattttcaggtggtattacacagacacgtgagtttattggtcttttatttatgccctgtctgaggatgcagattggtgggtagatgagaaggaactgattgagagagattaaccccaagaactgatatcttcccagcattgcattctcaactccattttagaaaggttccaaatagggacttctgtgggtttttctttacatccatcttacccttcccaagtccccatgtccctgcgtaaaccctaaagccacctctcaaaaggttctctagttcccttcaaggttctctagttcccttcattccacatatctcctcttccacaccctctagccagtagagctcccttctgacaagcaagtctaagatctagatgacagatgacttcctgcatttgggtggttcttttgtcactaatttgccttttctaaaattgtcctggtttcttctgccaatttcccttctttctccccagcatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagaggtaatccaggtctccagaacaggtaccaccggctctttagggaggaccattcaaaagggcattctcagtgattttccctaacccagctcacagtgcccaggcgtctttgcgcttcctcccacactcaatcctgggactctctggtaccacacggcatcagtgttttctggaatatagattaaacaccaatatgaggcttctgggtaaccccagtctgtgcgagatctaaaatagcaactccctaagagacaggactgggtcatttgcaccgcatcacacccaggttcatagcacaccaacatgagtttatctaatgcttcctccagagataaatttttcagaaaggtttgcaaaaaacactcaaggccactatagtaaaatggcataagctaaggtataataataaaataataacaatacttaacatttattgagtgcttattaagtctcaagcactgtctgtacccaacacttatcaaggattctttttcatgtaatcctctcaacaactatatgggttaagtatcattttattcccatgagtaaagggatgaggaaacagagggtttgtgagttgaaaacacatttcacgcttctcacagctagtgagtaataaagctgggactcaaacccagggctgtttgactccagtgcctctacccacggccaccactctttgcttgtcaatgttgttctaaacatattgaagggggggctctgaccgtggcaagcgtgtgagtagtaaggggagaatggccttcatgcactccctcctcacctccagcgccttgtgttttccttgcttagtgatttcccctctccccaccccaccccccacagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggtaaggctgtggagtccagtcagaggagattcctgccaagggggacgaccagcctgggccagggtgggtggcaagtccacagctaggtcagaacagcttctctagagcttctatgcacagcttctattactgtgatgacaagatctcaacagacggtttcaaatctcacatcactcccctccttcccatcctagaaaagtgcaaaaaagtttatgaaagtgatgggcttcctcacatacctgtcaatgcctgcagtcatccgattccgcccctaagctgtgggaagagagactttgatatattagctcctgccttttcctttcccttcccctatggagagaaacaatgggaggatcttgagctgaggaaagtcacaaaatgatgagaagagtgtagggtccttagagatgaatgaaagaaaaaaaaagagaaaggacgtctgaacagaaaagggagcggtagaggagagaacaatggggtttgccattctctatctgggtctcactggcacagacagtgctgcaagattggttccctcatgggaatgaaatgtttcccctccttcctccgcaggacaaaacaaggagaggccaccacctgttcccaacccagactatgaggtaacgtgggatagaaatgggccaggacgctggaggggatgtccctccaggggggaaggaaacagatgggatggcccatcttgtctgccagatgcctcaaagcccctcactcagggcttccattacaaccctctatgtgccacctctgcgtccttcatggtaaaacaggactgtctcaaaggctgcatggcttccacaaccatggagaggtggaagcttgcaggagacatactcctctttctctggcttattcattgactgggatacagccatggagaatattatatatgcaaattctaacacaataaattctgggctgatattccaccagcatgcaccagtatagcgagttattgaaatattaaaattatataaatattatataaaagttattgaaatattaaaatactcattgggaaatagccccaaactttgctcaccccaacccacccttacacacacacatacacacacacacacacacacacacacacacacacacacacgtgaccagacatcccagtccctcccctaccgggctgcctcttgagttggggtaacaaagagttaatgcctggcatggcagaggatcaccaggattgttctagttgattggtatgtgtgcactcctagttgttaaatattttcactatcacacctggatatactcaacaaatatttgttgagccaaatactcaacaccagccaaacacgtagtatttactttagcttaagcgaattatttagccctgacagaagccctggaatgtgggtctttaagttcctatttttgagatgggaaagctgaggctcacggaaggaggtgaccagctcaagtctcctaccgtccatgccaaattagaattccagcctgcctcctgacttcaagtccaaagttcttcccacgcactaaagctagctcttcagtgtcctttcttaggaggtacttcctcccgcaccactgaccgccccctctctatttcacccccagcccatccggaaaggccagcgggacctgtattctggcctgaatcagagacgcatctga

SEQ ID NO: 15 - DNA, 100 bp, Artificial Sequence
  connection sequence
ggaaaggccagcgggacctgtattctggcctgaatcagagacgcatctgacagataggagagacatcgccttctgtggacccagatccagccctccgagc

SEQ ID NO: 16 - DNA, 100 bp, Artificial Sequence
  connection sequence
tggtgaggaaggaattggggctgagaagctggtttccatcagctgccaggaagcttgatatcgaattccgaagttcctattctctagaaagtataggaac

SEQ ID NO: 17 - DNA, 100 bp, Artificial Sequence
  connection sequence
aaagtataggaacttcatcagtcaggtacataatggtggatccagaattcaagtgctgctgaacagagccagggaagacgagaaaagccgtgcgtgcgtg

SEQ ID NO: 18 - DNA, 1511 bp, Artificial Sequence
  the CD3E mRNA sequence of the transformed humanized mouse
agacacctgcctcccctgtctgcgtctggtgccttcttcagaaatgaagtaatgagctggctgcgtccgccatcttggtagagagagcattctgagaggatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgacagataggagagacatcgccttctgtggacccagatccagccctccgagcaccctgctactccttgttctctggacagactgcagactccacagcttgctcttcagcctcctggtgaacacgtgtcctagaaccttgctctcctgcctcctctgctagtagccagtgctgggacattgctgactcaacagcctttgaaagaatcaggctgctcagattgtctgccagccaccttgtggggatacttttttcagccgccctgctgccagctccccgctgcctcaccagtgtcctctctgcctcagttcctttcctctcctaattggccctcatagctaagccctttcctacagctttctgttttttctttttctttctttttaggttttctttcttttcttttttattcctttttatttaatcttttttttttaaacactccagattttattcccttcccggcccatcctccgactgttacacaccccatacctcctccctgcccccttgtctctacgagaatgtcccccacccccatcccccacctgctttctggtttttggtttttggtttgttttttttttttaaactctgtgttttacactcttctctgggatggattctgtaatcattggcacaggtcctgccccatttatagatcctggcccagcccctgccacaggtgcctctccagatttccccttagatcctcggatggtcatctccatctccatgaatacaccagccccctctctgctaatgcaaaaggcaataaagtgtattggctgg

SEQ ID NO: 19 - DNA, 27 bp, Artificial Sequence
  primer
acattgtgcaacatggttcctttatga

SEQ ID NO: 20 - DNA, 25 bp, Artificial Sequence
  primer
ggcaccattttagcttcctttcgcc

SEQ ID NO: 21 - DNA, 25 bp, Artificial Sequence
  primer
ctcgactgtgccttctagttgccag

SEQ ID NO: 22 - DNA, 27 bp, Artificial Sequence
  primer
acccaaattccttgttttcagttgcca

SEQ ID NO: 23 - DNA, 25 bp, Artificial Sequence
  primer
gagctacacagtgagactaccacag

SEQ ID NO: 24 - DNA, 26 bp, Artificial Sequence
  primer
cacagtatctgacaataaatggcaac

SEQ ID NO: 25 - DNA, 20 bp, Artificial Sequence
  primer
gctcgactagagcttgcgga

SEQ ID NO: 26 - DNA, 27 bp, Artificial Sequence
  primer
ctctcagtaatcaacttctcgtgtgtc

SEQ ID NO: 27 - DNA, 22 bp, Artificial Sequence
  probe
tacagaacacatggaggcacag

SEQ ID NO: 28 - DNA, 22 bp, Artificial Sequence
  probe
gtaacaacgctgcaacaggatc

SEQ ID NO: 29 - DNA, 25 bp, Artificial Sequence
  probe
cacactctcatatcactgtacacac

SEQ ID NO: 30 - DNA, 23 bp, Artificial Sequence
  probe
tgatggatccaagagtgggcaac

SEQ ID NO: 31 - DNA, 25 bp, Artificial Sequence
  probe
tcgatgtaggagggcgtggatatgt

SEQ ID NO: 32 - DNA, 25 bp, Artificial Sequence
  probe
tgtattgaccgattccttgcggtcc

SEQ ID NO: 33 - DNA, 25 bp, Artificial Sequence
  probe
gcttcaaggatgtttaactgcaaag

SEQ ID NO: 34 - DNA, 20 bp, Artificial Sequence
  probe
tgcaacagatgtgctggcga

SEQ ID NO: 35 - DNA, 70 bp, Artificial Sequence
  connection sequence
tggtacgtgcttgtaatcctaacactgtgagatatcgtggctcatgcctctaatcccagcacttttggag

SEQ ID NO: 36 - DNA, 72 bp, Artificial Sequence
  connection sequence
gctgcgtccgccatcttggtagagagagcattctgagaggatgcagtcgggcactcactggagagttctggg

SEQ ID NO: 37 - DNA, 21 bp, Artificial Sequence
  probe
ggatcggccattgaacaagat

SEQ ID NO: 38 - DNA, 22 bp, Artificial Sequence
  probe
cagaagaactcgtcaagaaggc

SEQ ID NO: 39 - DNA, 24 bp, Artificial Sequence
  primer
gatccacaccattgcacaagccag

SEQ ID NO: 40 - DNA, 25 bp, Artificial Sequence
  primer
tgctttgagttgttggttgagtttc

SEQ ID NO: 41 - DNA, 25 bp, Artificial Sequence
  primer
aaatggggatcagaaacccttcacc

SEQ ID NO: 42 - DNA, 22 bp, Artificial Sequence
  primer
atggcaaccaatgatccagggt

SEQ ID NO: 43 - DNA, 20 bp, Artificial Sequence
  primer
ctgagtccccagcccttgtc

SEQ ID NO: 44 - DNA, 21 bp, Artificial Sequence
  primer
atgaggctccttggtgccact

SEQ ID NO: 45 - DNA, 25 bp, Artificial Sequence
  primer
gcagtgcttcctgttagaggaggtg

SEQ ID NO: 46 - DNA, 25 bp, Artificial Sequence
  primer
ttctcgtcttccctggctctgttca

SEQ ID NO: 47 - DNA, 26 bp, Artificial Sequence
  primer
tgttgggcacttgtatttgcatttgt

SEQ ID NO: 48 - DNA, 25 bp, Artificial Sequence
  primer
gggattgggctcaggttgtcacatt

SEQ ID NO: 49 - DNA, 25 bp, Artificial Sequence
  primer
gacgtgccctctagacagtgacgag

SEQ ID NO: 50 - DNA, 25 bp, Artificial Sequence
  primer
acacactcgagctttcaggtacaag

SEQ ID NO: 51 - DNA, 25 bp, Artificial Sequence
  primer
taggcagtgatgaggatcacctgtc

SEQ ID NO: 52 - DNA, 25 bp, Artificial Sequence
  primer
cttgttttgtcccctttgcctgccg

SEQ ID NO: 53 - DNA, 25 bp, Artificial Sequence
  primer
aacggtggaaggatggtttgcaaag

SEQ ID NO: 54 - DNA, 25 bp, Artificial Sequence
  primer
gcttgaacctcagcagccccagaag

SEQ ID NO: 55 - DNA, 25 bp, Artificial Sequence
  primer
aattgcaataccagcatcacatggg

SEQ ID NO: 56 - DNA, 25 bp, Artificial Sequence
  primer
atctcggaggggctgatagacctgg

SEQ ID NO: 57 - DNA, 25 bp, Artificial Sequence
  primer
agcccagacaaataaagcaaagaat

SEQ ID NO: 58 - DNA, 25 bp, Artificial Sequence
  primer
gtcccgcaatgagatatacaccaag

SEQ ID NO: 59 - DNA, 25 bp, Artificial Sequence
  primer
ggcccagtcaatcaaaggaaaccac

SEQ ID NO: 60 - DNA, 25 bp, Artificial Sequence
  primer
ggagtggttttgacttgttctgtga

SEQ ID NO: 61 - DNA, 22 bp, Artificial Sequence
  primer
tcaccatcttccaggagcgaga

SEQ ID NO: 62 - DNA, 21 bp, Artificial Sequence
  primer
gaaggccatgccagtgagctt

SEQ ID NO: 63 - DNA, 8780 bp, Homo sapiens
atgcagtcgggcactcactggagagttctgggcctctgcctcttatcaggtgagtaggatggagtggaaagggtggtgtgtctccagaccgctggaaggcttacagccttacctggcactgcctagtggcaccaaggagcctcatttaccagatgtaaggaactgtttgtgctatgttagggtgagggattagagctggggactaaagaaaaagataggccacgggtgcctgggagagcgttcggggagcaggcaaagaagagcagttggggtgatcatagctattgtgagcagagaggtctcgctacctctaagtacgagctcattccaacttacccagccctccagaactaacccaaaagagactggaagagcgaagctccactccttgttttgaagagaccagatacttgcgtccaaactctgcacagggcatatatagcaattcactatctttgagaccataaaacgcctcgtaatttttagtccttttcaagtgaccaacaactttcagtttatttcatttttttgaagcaagatggattatgaattgataaataaccaagagcatttctgtatctcatatgagataaataataccaaaaaaagttgccatttattgtcagatactgtgtaaagaaaaaattatttagacgtgttaactggtttaatcctacttctgcctaggaaggaaggtgttatatcctctttttaaaattctttttaattttgactatataaactgataagtcctctctacttcacagattaagaaattgatactcaaaaaagttaaataacttgttttaaaccacatagtaagtgccgaagccaatctgtgagaccaggactgtttgtactctaaatggctgcaccacatgaggcaaaatggctcgtgatggttttatttcaaagacctagaaaacactatcacagctggtgctcccgtctcagacccacagcaacgatgtctccacttcctgcttcatcttgggtttctcacgtcttgaatgtgcacacaaatcacctggggatcttgttaaaaatgcagcttctgtttcaaaagacccagactggaaatggagattccgcatgtctagtacgctctcaagtttattaatctgctgctggtcctaggaacatattgagtagcgaggggcaggatgtgactcctgtaaggagtggccaggcattttcttagagacctgtgttataaagtatgcttttccttaaaaaaaagaagaaggaggaggaggagaagggccaggtgcagtggcttacgcctgtaagcccagcattttgggggaccaaagtgggaagatcacttgagctcaggagttcaagaccagcctgggcaaagtagtgaaaccccatttctacaaaaaattaaaaattagccaggcaaggtggcacacacctatagttccagctacttgggaggctgaggtggaaggatcacttgagcccaggaggtcgaggctgcagtaagccatgatcaagccactgcactccagcctggagtgtctcaaaatagataaataaataaataaataaataaataaataaataaataaaagagacagtatcaaagacccaatcacctctagacatctggcatcataggaatgtgcccagtctgctctggggataggaaagtggggatcctgtctccccctgtgtagaggtttcagtaaaagaaaggcctaggtgtgcagaaagctttcaggcaatgccagggaaactgatcattgtaatgaatccagggtattgctgagtgagggcatcctggagggcccggtggaaatgtggtcaggctcttcaatgcacaggccctagttgatgagtaatcagggtttcaaatatttccatctctgtctcaagcagaaaacaaatggaaaaactgaaccaccagaaaagcagagccagagatggaacaagaatcccagtgtttgtacccaaccaagagcgtgtttttcttccacagacaccaatgttcaaaatggaggcttgggggcaaaattcttttgctatgtctctagtcgtccaaaaaatggtcctaactttttctgactcctgcttgtcaaaaattgtgggctcatagttaatgctagatgcttccttcctctatttccccccaaatttcctgggaacccctggtcaataccagcagtaagttccactgttctagggtgtagaaatggctgtgacccagcagcaagagggaaggacatcagatgtcatcagtggtcatactgcaacacagccctttttctgtttaggaatgcaggtacccacaacatttactaacactttttttttcttatttattttctagttggcgtttgggggcaagatggtgagatatgctttctttctttcttttttatgaaatcaccccatcattctttgtagttatgaatggagctttctcttaggcctcccacagaacttccacagaggtcaggaaaaggagtttctgccatctacccctttgactttcctcacaagtctggagatatttctagcccagaagagggaagcaacagaggcaggaaataatgagtcttaaccatacaaaagaaaaattgagacttaaatgaagttgaaagcactaacagttttcatttgtttgcatttcatatttgatgtgagattctgcagaggagacgtagccagaatgcatgcacagggttactctggataagctgctggggcaacatttggatgtgtgttcagaatcacatgtctgaatactctgaatatatgtgtgtacatgtgtatttatgcaagtgcacatgcatatgagtgtgcccggcctgaacttactctctcaaccacagcggtagagtcaggagtgttccaacattggaagcccctctattcaatcagctcttccaaactgagtgaaccaatgttgtatttaatggcaaccatggctggacaccatggctcacacctgtaatcccagcactttgggaggccgaggtgggcagatcacttgaggccaggagttcgagaccagcctggccaacatggcgaaaccccgtctctactaaaaataccaaaatcagccagacatggtggtgtacgcctgtagtcctagctactcgagaagctgaggcaggagaatcgcttgaacctgggaggcagaggttgtagtgagccgagatcacaccactgcactctagcctgggtgacacatcgagactgtctcaaaaataaaataaagacaaccattatgccagcctagattccgccatgctgcctaatttgtagtgtccttaggagccatttttgtaaatagtcatcagataagatgtaaggcccataacagctttttctatgcagctgagggaattggaagatccattgtttcctaagagttgagggaagagtcccaacccacgggagcagggtctgatcttcattgccgatagaaacattactaatggcttcttactgtttccttttcaggtaatgaagaaatgggtaagaagatttccactctatctagcaaaagttttcaaatatggaatgaaatgctcatagagtacaatcacagtaacaaaccctgagaactaaaactattaaagggaaaatacaagtatctttcaatgggatccgtatgaaacttgcctgtatttgttgctagctgtcatgtcagattatagctgtgcatatatgtatctctgatcatacacatatggatgtgggttggagctaccatgtgtttttgtataagccatgaaatctttgaaggcagacagagacagtgtctcatttacctagcccagtgtctggcacatagtaggtgctcaatgaatattttttgaatgaataaatgaacaaacatatgaacacattgctaattacctcccctcaagaagctgatggtcttgtgtgagagacaaataattgaaaatatagtgagttgcatgttataatatgggtagatacagagtaaaatgaagtataaagaggggagtggtcaactctactgagtgtcgttgggaaaggttccctgggggaggtggtccttgagctgaattttaaaggataagcttatgttttagggaagaaaaatattttatgcagaagagataaagctgtatagtatgaggataagagtctaactgagctagatcagaatgtttgaatcttggctcaactctctacttgctgggtgtgtttgagtaatttacctaacttttctgtgccacagcatcatcatggtacaatggaaataatagtgctacctaacttgtagggttattatgaggaccaaatgagtaattcatttaaggcacttagaacattatctgacataaaaggcagtaggagggccgggcatggtggctcacacctgtaatcccagcactttgagaagccgaggtgggaggatcacctgaggtcaggagttcgagaccggcctggccaacatggtgaaactccatctctgctaaaaatacaaaaattagccaggcatggtggcaggtgcctgtaatctcagctactcaggaggctgaggtaggagaattgcttgaacctgggaggcggaggttggagtgagctgagattgtgccattgcactccagcctgggcgacagagcaagactctgtctcaaaacaaacaaacaaacaaacagacagtaggtgaattttagctattaatacatggaaagcatgctgactatagatgataagcattaaagtttactgagcatgtatgttttaggcattgctctaaatattttacttgaatttcctcatttaattcttccaacacccctactgtacagttaaggaaacaaagcctcaaataaatacagaaataaacaaaaataagtaaacaatccagtcctggggatataaatgcagatttaggccaagtgccatggttcatgcctataatcccaacactttgggaggccaaggcaggaggctcgcttgagctcagaaggttgaggctgcattgagcaaagattgtgccactgtactcgagcctctgtggcagagaaagaccctgtctctgaaaaaattaataaatagaaatttaaaaataaaaaaattttaatgcagatttatatgataccgaagttcattttctcaaccattatgaaatactgtttctggatatgtataaaatctttgtgagcacacatatctttttttaacttaactttcattttaaattcaggggtacatgtgcaggtttgttatataggtaaacttgtgccatgggggtttgttgtatagattatttcatcacccaggcattaagcctggtacctgttagttatttttcctgatcctctccctcctcccaccctccaccttctgagaggtcccagtgtgtgtcatttccctctgtgttcatgtgttctcatcatttagctcccacttctaaatgagaacatgtggtatttggttttctgtcactgtgttagtttgctaaagataatggcctccagtcccatccatgttcctgcaaaggacgtgatctcattcttttttatggctgcgtagtattccatggtgtatatgcagcacatttttttatccagtctaccactgacaggcatttaggttgattccatgtctttgctattgtgaatagtgcacaatgaacatacgtggagcacatttctgtctaagcacagacatctagacccttgtgtgagcatgagttaagtctaagctctgctactgaatttgtgccaataaaagttgtgagcaattttctttacatttttttcaaacaaacacacccagcagagtataatgtctatgtactttatttatgatttctagttcatttaacatgtctaagaaacatccgtgttgaaaaattatttataaattaaaataatataaactatctactgtccttatactcaactcccaattataagcaggtggaaaaacctggagaatgttttgtttacattctgtgcagtctttgtcagagggctgcctgagcaactgggtcagagtttagttctgctctgggagtagcaggacctcaagaaggaaaggaggaaaggaagtaactttttcttgagcacctgctatgtgtcattcactttcaccttcataatccatttaattttcgcaaaaactttgtgaggttggtgttttatctccatttccctgataaagaagttgaggttcagcaaagttaaatgacttgccctcagtcacacagactagggcagatccaggattcaaactcagggcttctgactcttgagtccagagctctgtccctgacagcagcagcactgcctctcctctcttccagctgttatgtccagactgtagcagaacccagtgttccagccacaagttttccaggaaataataaaggactcctagctccacctcccagggcaaaaatggctgctgtgggaaacacaggctggacctacgaatggcattagtggtttattagttgatttcagttgtccacactaataggcctccctctaacaaaaataattgagagctgattatgctcagatataatgtaaagtgaagccactttttattggaagaagcattccctcaaaacgtgtagagtatttcacattatttaaaggcaaatagagagaaaattatatggaataagaacaaagatgtttcttctctattatgagggactcagttctgagaaaggattttaaattgtaagaaataggtaagtccacgaatcagtgattcagtggtgtggagagctttatttctgagaaggccagtagcgctcccttctgacaagcaaatctaagacctggatgacagatgacttcctgcatttggttggttcttttgtcattcatatctatctgtaatacagttctggctaatttaagaggataagcttgaagacctctggaatttttcggctttaggactttaaggctttctgagcttcagtagatctagatctaggagctcatgctggtatattctgaatccgatgtatctgagttacatctatgagctacttaataaatatatctatgagctaaatctcataggctaagcatgaacctcacctccaagactcggggttcctaaatggatgagaccctctttgggaagtcttgtgggcagtgtctaattccactagaaaagttttacctacaatttaaacttaaaccatgatattttcttactgctgtttccttttttcattttcaggtggtattacacagacacgtgagtttattggtcttttatttatgccctgtctgaggatgcagattggtgggtagatgagaaggaactgattgagagagattaaccccaagaactgatatcttcccagcattgcattctcaactccattttagaaaggttccaaatagggacttctgtgggtttttctttacatccatcttacccttcccaagtccccatgtccctgcgtaaaccctaaagccacctctcaaaaggttctctagttcccttcaaggttctctagttcccttcattccacatatctcctcttccacaccctctagccagtagagctcccttctgacaagcaagtctaagatctagatgacagatgacttcctgcatttgggtggttcttttgtcactaatttgccttttctaaaattgtcctggtttcttctgccaatttcccttctttctccccagcatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagaggtaatccaggtctccagaacaggtaccaccggctctttagggaggaccattcaaaagggcattctcagtgattttccctaacccagctcacagtgcccaggcgtctttgcgcttcctcccacactcaatcctgggactctctggtaccacacggcatcagtgttttctggaatatagattaaacaccaatatgaggcttctgggtaaccccagtctgtgcgagatctaaaatagcaactccctaagagacaggactgggtcatttgcaccgcatcacacccaggttcatagcacaccaacatgagtttatctaatgcttcctccagagataaatttttcagaaaggtttgcaaaaaacactcaaggccactatagtaaaatggcataagctaaggtataataataaaataataacaatacttaacatttattgagtgcttattaagtctcaagcactgtctgtacccaacacttatcaaggattctttttcatgtaatcctctcaacaactatatgggttaagtatcattttattcccatgagtaaagggatgaggaaacagagggtttgtgagttgaaaacacatttcacgcttctcacagctagtgagtaataaagctgggactcaaacccagggctgtttgactccagtgcctctacccacggccaccactctttgcttgtcaatgttgttctaaacatattgaagggggggctctgaccgtggcaagcgtgtgagtagtaaggggagaatggccttcatgcactccctcctcacctccagcgccttgtgttttccttgcttagtgatttcccctctccccaccccaccccccacagtgtgtgagaactgcatggagatggat

SEQ ID NO: 64 - DNA, 69 bp, Artificial Sequence
  connection sequence
ccacagtgtgtgagaactgcatggagatggatctgacagcagtagccataatcatcattgttgacatct

SEQ ID NO: 65 - DNA, 1490 bp, Artificial Sequence
  the CD3E mRNA sequence of the transformed humanized mouse
gcgtctggtgccttcttcagaaatgaagtaatgagctggctgcgtccgccatcttggtagagagagcattctgagaggatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatctgacagcagtagccataatcatcattgttgacatctgtatcactctgggcttgctgatggtcatttattactggagcaagaataggaaggccaaggccaagcctgtgacccgaggaaccggtgctggtagcaggcccagagggcaaaacaaggagcggccaccacctgttcccaacccagactatgagcccatccgcaaaggccagcgggacctgtattctggcctgaatcagagagcagtctgacagataggagagacatcgccttctgtggacccagatccagccctccgagcaccctgctactccttgttctctggacagactgcagactccacagcttgctcttcagcctcctggtgaacacgtgtcctagaaccttgctctcctgcctcctctgctagtagccagtgctgggacattgctgactcaacagcctttgaaagaatcaggctgctcagattgtctgccagccaccttgtggggatacttttttcagccgccctgctgccagctccccgctgcctcaccagtgtcctctctgcctcagttcctttcctctcctaattggccctcatagctaagccctttcctacagctttctgttttttctttttctttctttttaggttttctttcttttcttttttattcctttttatttaatcttttttttttaaacactccagattttattcccttcccggcccatcctccgactgttacacaccccatacctcctccctgcccccttgtctctacgagaatgtcccccacccccatcccccacctgctttctggtttttggtttttggtttgttttttttttttaaactctgtgttttacactcttctctgggatggattctgtaatcattggcacaggtcctgccccatttatagatcctggcccagcccctgccacaggtgcctctccagatttccccttagatcctcggatggtcatctccatctccatgaatacaccagccccctctctgctaatgcaaaaggcaataaagtgtattggctgg

SEQ ID NO: 66 - protein, 207 aa, Artificial Sequence
  the CD3E protein sequence of the transformed humanized mouse
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDLTAVAIIIIVDICITLGLLMVIYYWSKNRKAKAKPVTRGTGAGSRPRGQNKERPPPVPNPDYEPIRKGQRDLYSGLNQRAV

(19) United States
(12) Patent Application Publication
(10) Pub. No.: US 2024/0130340 A1
(43) Pub. Date: Apr. 25, 2024

(54) GENETICALLY MODIFIED NON-HUMAN ANIMAL WITH HUMAN OR CHIMERIC CD3 GENES

(71) Applicant: Biocytogen Pharmaceuticals (Beijing) Co., Ltd., Beijing (CN)

(72) Inventors: Chengzhang Shang, Beijing (CN); Chong Li, Beijing (CN); Chang Liu, Beijing (CN)

(21) Appl. No.: 18/277,394

(22) PCT Filed: Feb. 18, 2022
(86) PCT No.: PCT/CN2022/076966

§ 371 Date: Aug. 16, 2023

(30) Foreign Application Priority Data
Feb. 19, 2021 (CN) 202110189578.6

(51) Int. Cl. A01K 67/0278; C07K 14/725

(52) U.S. Cl. CPC A01K 67/0278; C07K 14/7051; A01K 2207/15; A01K 2227/105; A01K 2267/0331

(57) ABSTRACT

Provided are genetically modified non-human animals that express a human or chimeric (e.g., humanized) CD3E (T-cell surface glycoprotein CD3 epsilon chain), CD3D (T-cell surface glycoprotein CD3 delta chain), and/or CD3G (T-cell surface coprotein CD3 gamma chain), and methods of use thereof.

### CLAIM OF PRIORITY

This application claims the benefit of Chinese Patent Application App. No. 202110189578.6, filed on Feb. 19, 2021. The entire contents of the foregoing application are incorporated herein by reference.

### TECHNICAL FIELD

This disclosure relates to genetically modified animal expressing human or chimeric (e.g., humanized) CD3E, CD3D, and/or CD3G, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure relates to transgenic non-human animal with human or chimeric (e.g., humanized) CD3E (T-cell surface glycoprotein CD3 epsilon chain; also known as CD3E or T-Cell Surface Antigen T3/Leu-4 Epsilon Chain), human or chimeric CD3D (T-Cell Surface Glycoprotein CD3 Delta Chain; also known as CD36), human or chimeric CD3G (T-Cell Surface Glycoprotein CD3 Gamma Chain; also known as CD3γ) and methods of use thereof.

The animal model can express human CD3E or chimeric CD3E (e.g., humanized CD3E) protein, human CD3D or chimeric CD3D (e.g., humanized CD3D) protein, and/or human CD3G or chimeric CD3G (e.g., humanized CD3G) protein in its body. It can be used in the studies on the function of CD3E, CD3D, and/or CD3G genes, and can be used in the screening and evaluation of anti-human CD3E, CD3D, CD3G antibodies. In addition, the animal models prepared by the methods described herein can be used in drug screening, pharmacodynamics studies, treatments for immune-related diseases (e.g., autoimmune disease), and cancer therapy for human CD3E, CD3D, and/or CD3G target sites; they can also be used to facilitate the development and design of new drugs, and save time and cost. In summary, this disclosure provides a powerful tool for studying the function of CD3E, CD3D, CD3G proteins, and a platform for screening cancer drugs.

In one aspect, the disclosure is related to a genetically-modified, non-human animal whose genome comprises at least one chromosome comprising a sequence encoding a human or chimeric CD3D. In some embodiments, the sequence encodes a human CD3D. In some embodiments, the sequence encoding the human or chimeric CD3D is operably linked to a human regulatory element (e.g., 5′ UTR and/or 3′ UTR) at an endogenous CD3D gene locus in the at least one chromosome. In some embodiments, the human or chimeric CD3D comprises an amino acid sequence that is at least 70%, 75%, 80%, 85%, 90%, 95%, 96%, 97%, 98%, 99%, or 100% identical to human CD3D (NP_000723.1 (SEQ ID NO: 4)). In some embodiments, the animal is a mammal, e.g., a monkey, a rodent, or a mouse. In some embodiments, the animal is a mouse. In some embodiments, the animal does not express endogenous CD3D. In some embodiments, the animal has one or more cells expressing human or chimeric CD3D. In some embodiments, the animal has one or more cells expressing human CD3D, in some embodiments, the expressed human CD3D can form a CD3 complex with human or humanized CD3E, human or humanized CD3G and endogenous CD3-zeta polypeptides.

In one aspect, the disclosure is related to a genetically-modified, non-human animal, in some embodiments, the genome of the animal comprises a replacement of a sequence encoding a region of endogenous CD3D with a sequence encoding a corresponding region of human CD3D at an endogenous CD3D gene locus. In some embodiments, the sequence encoding the corresponding region of human CD3D is operably linked to a human regulatory element (e.g., 5′ UTR and/or 3′ UTR) at the endogenous CD3D locus, and one or more cells of the animal expresses human or chimeric CD3D. In some embodiments, the animal does not express endogenous CD3D. In some embodiments, the sequence that is replaced encodes full-length endogenous CD3D. In some embodiments, the animal is a mouse, and the sequence that is replaced comprises exon 1, exon 2, exon 3, exon 4, and/or exon 5 of the endogenous mouse CD3D gene. In some embodiments, the sequence that is replaced further comprises 5′ UTR and/or 3′ UTR of the endogenous mouse CD3D gene. In some embodiments, the animal is heterozygous with respect to the replacement at the endogenous CD3D gene locus. In some embodiments, the animal is homozygous with respect to the replacement at the endogenous CD3D gene locus.

In one aspect, the disclosure is related to a non-human animal comprising a nucleotide sequence encoding a chimeric CD3D polypeptide, in some embodiments, the chimeric CD3D polypeptide comprises a human CD3D signal peptide, and at least 50 contiguous amino acid residues that are identical to the corresponding contiguous amino acid sequence of a human CD3D, in some embodiments, the animal expresses the chimeric CD3D polypeptide. In some embodiments, the chimeric CD3D polypeptide has at least 50, at least 60, at least 70, at least 80, at least 90, at least 100, at least 110, at least 120, at least 130, at least 140, at least 150, at least 160, or at least 170 contiguous amino acid residues that are identical to the corresponding contiguous amino acid sequence of a human CD3D. In some embodiments, the chimeric CD3D polypeptide comprises a sequence that is at least 90%, 95%, 96%, 97%, 98%, 99%, or 100% identical to SEQ ID NO: 4. In some embodiments, the nucleotide sequence is integrated to an endogenous CD3D gene locus of the animal. In some embodiments, the animal further comprises a sequence encoding an additional human or chimeric protein. In some embodiments, the additional human or chimeric protein is CD3E, CD3G programmed cell death protein 1 (PD-1), Programmed Cell Death 1 Ligand 1 (PD-L1), cytotoxic T-lymphocyte-associated protein 4 (CTLA-4), Lymphocyte Activating 3 (LAG-3), B And T Lymphocyte Associated (BTLA), tumor necrosis factor receptor superfamily member 9 (4-1BB), T-Cell Immunoreceptor With Ig And ITIM Domains (TIGIT), B7 Homolog 3 (B7-H3), T-cell Immunoglobulin and Mucin-Domain Containing-3 (TIM-3), Glucocorticoid-Induced TNFR-Related Protein (GITR), TNF Receptor Superfamily Member 4 (OX40), CD27, CD28, CD38, CD47, CD154, CD40, CD278, or CLDN18.2.

In one aspect, the disclosure is related to a method for making a genetically-modified, non-human animal, comprising: replacing in at least one cell of the animal, at an endogenous CD3D gene locus, a sequence encoding a region of an endogenous CD3D with a sequence encoding a corresponding region of human CD3D. In some embodiments, the sequence encoding the corresponding region of human CD3D comprises exon 1, exon 2, exon 3, exon 4, and/or exon 5, or a part thereof, of a human CD3D gene. In some embodiments, the sequence encoding the corresponding region of human CD3D encodes SEQ ID NO: 4. In some embodiments, the animal is a mouse, and the endogenous CD3D locus comprises exon 1, exon 2, exon 3, exon 4, and/or exon 5, or a part thereof, of the mouse CD3D gene.

In one aspect, the disclosure is related to a method of making a genetically-modified non-human animal (e.g., mouse) cell that expresses a human or chimeric CD3D, the method comprising: replacing at an endogenous CD3D gene locus, a nucleotide sequence encoding a region of endogenous CD3D with a nucleotide sequence encoding a corresponding region of human CD3D, thereby generating a genetically-modified non-human animal cell that includes a nucleotide sequence that encodes the human or chimeric CD3D, in some embodiments, the non-human animal cell expresses the human or chimeric CD3D. In some embodiments, the nucleotide sequence encoding the human or chimeric CD3D is operably linked to a human CD3D regulatory region, e.g., promoter. In some embodiments, the nucleotide sequence encoding the human or chimeric CD3D is operably linked to an endogenous CD3D regulatory region, e.g., promoter. In some embodiments, the animal further comprises a sequence encoding an additional human or chimeric protein. In some embodiments, the additional human or chimeric protein is CD3E, CD3G PD-1, PD-L1, CTLA-4, LAG-3, BTLA, 4-1BB, TIGIT, B7-H3, TIM-3, GITR, OX40, CD27, CD28, CD38, CD47, CD154, CD40, CD278, and/or CLDN18.2.

In one aspect, the disclosure is related to a genetically-modified, non-human animal whose genome comprises at least one chromosome comprising a sequence encoding a human or chimeric CD3G In some embodiments, the sequence encodes a human CD3G In some embodiments, the sequence encoding the human or chimeric CD3G is operably linked to a human regulatory element (e.g., 5′ UTR and/or 3′ UTR) at an endogenous CD3G gene locus in the at least one chromosome. In some embodiments, the human or chimeric CD3G comprises an amino acid sequence that is at least 70%, 75%, 80%, 85%, 90%, 95%, 96%, 97%, 98%, 99%, or 100% identical to human CD3G (NP_000064.1 (SEQ ID NO: 6)). In some embodiments, the animal is a mammal, e.g., a monkey, a rodent, or a mouse. In some embodiments, the animal is a mouse. In some embodiments, the animal does not express endogenous CD3G In some embodiments, the animal has one or more cells expressing human or chimeric CD3G In some embodiments, the animal has one or more cells expressing human CD3G in some embodiments, the expressed human CD3G can form a CD3 complex with human or humanized CD3E, human or humanized CD3D, and endogenous CD3-zeta polypeptides.

In one aspect, the disclosure is related to a genetically-modified, non-human animal, in some embodiments, the genome of the animal comprises a replacement of a sequence encoding a region of endogenous CD3G with a sequence encoding a corresponding region of human CD3G at an endogenous CD3G gene locus. In some embodiments, the sequence encoding the corresponding region of human CD3G is operably linked to a human regulatory element (e.g., 5′ UTR and/or 3′ UTR) at the endogenous CD3G locus, and one or more cells of the animal expresses human or chimeric CD3G In some embodiments, the animal does not express endogenous CD3G In some embodiments, the sequence that is replaced encodes full-length endogenous CD3G In some embodiments, the animal is a mouse, and the sequence that is replaced comprises exon 1, exon 2, exon 3, exon 4, exon 5, exon 6, and/or exon 7 of the endogenous mouse CD3G gene. In some embodiments, the sequence that is replaced further comprises 5′ UTR and/or 3′ UTR of the endogenous mouse CD3G gene. In some embodiments, the animal is heterozygous with respect to the replacement at the endogenous CD3G gene locus. In some embodiments, the animal is homozygous with respect to the replacement at the endogenous CD3G gene locus.

In one aspect, the disclosure is related to a non-human animal comprising a nucleotide sequence encoding a chimeric CD3G polypeptide, in some embodiments, the chimeric CD3G polypeptide comprises a human CD3G signal peptide, and at least 50 contiguous amino acid residues that are identical to the corresponding contiguous amino acid sequence of a human CD3G in some embodiments, the animal expresses the chimeric CD3G polypeptide. In some embodiments, the chimeric CD3G polypeptide has at least 50, at least 60, at least 70, at least 80, at least 90, at least 100, at least 110, at least 120, at least 130, at least 140, at least 150, at least 160, at least 170, or at least 180 contiguous amino acid residues that are identical to the corresponding contiguous amino acid sequence of a human CD3G In some embodiments, the chimeric CD3G polypeptide comprises a sequence that is at least 90%, 95%, 96%, 97%, 98%, 99%, or 100% identical to SEQ ID NO: 6. In some embodiments, the nucleotide sequence is integrated to an endogenous CD3G gene locus of the animal. In some embodiments, the animal further comprises a sequence encoding an additional human or chimeric protein. In some embodiments, the additional human or chimeric protein is CD3E, CD3D, programmed cell death protein 1 (PD-1), Programmed Cell Death 1 Ligand 1 (PD-L1), cytotoxic T-lymphocyte-associated protein 4 (CTLA-4), Lymphocyte Activating 3 (LAG-3), B And T Lymphocyte Associated (BTLA), tumor necrosis factor receptor superfamily member 9 (4-1BB), T-Cell Immunoreceptor With Ig And ITIM Domains (TIGIT), B7 Homolog 3 (B7-H3), T-cell Immunoglobulin and Mucin-Domain Containing-3 (TIM-3), Glucocorticoid-Induced Related Protein (GITR), TNF Receptor Superfamily Member 4 (OX40), CD27, CD28, CD38, CD47, CD154, CD40, CD278, or CLDN18.2.

In one aspect, the disclosure is related to a method for making a genetically-modified, non-human animal, comprising: replacing in at least one cell of the animal, at an endogenous CD3G gene locus, a sequence encoding a region of an endogenous CD3G with a sequence encoding a corresponding region of human CD3G In some embodiments, the sequence encoding the corresponding region of human CD3G comprises exon 1, exon 2, exon 3, exon 4, exon 5, exon 6, and/or exon 7, or a part thereof, of a human CD3G gene. In some embodiments, the sequence encoding the corresponding region of human CD3G encodes SEQ ID NO: 6. In some embodiments, the animal is a mouse, and the endogenous CD3G locus comprises exon 1, exon 2, exon 3, exon 4, exon 5, exon 6, and/or exon 7, or a part thereof, of the mouse CD3G gene.

In one aspect, the disclosure is related to a method of making a genetically-modified non-human animal (e.g., mouse) cell that expresses a human or chimeric CD3G the method comprising: replacing at an endogenous CD3G gene locus, a nucleotide sequence encoding a region of mouse CD3G with a nucleotide sequence encoding a corresponding region of human CD3G thereby generating a genetically-modified non-human animal cell that includes a nucleotide sequence that encodes the human or chimeric CD3G in some embodiments, the non-human animal cell expresses the human or chimeric CD3G In some embodiments, the nucleotide sequence encoding the human or chimeric CD3G is operably linked to a human CD3G regulatory region, e.g., promoter. In some embodiments, the nucleotide sequence encoding the human or chimeric CD3G is operably linked to an endogenous CD3G regulatory region, e.g., promoter. In some embodiments, the animal further comprises a sequence encoding an additional human or chimeric protein. In some embodiments, the additional human or chimeric protein is CD3E, CD3D, PD-1, PD-L1, CTLA-4, LAG-3, BTLA, 4-1BB, TIGIT, B7-H3, TIM-3, GITR, OX40, CD27, CD28, CD38, CD47, CD154, CD40, CD278, and/or CLDN18.2.

In one aspect, the disclosure is related to a genetically-modified, non-human animal whose genome comprises at least one chromosome comprising: (a) a sequence encoding a human or chimeric CD3E; (b) a sequence encoding a human or chimeric CD3D; and/or (c) a sequence encoding a human or chimeric CD3G In some embodiments, the sequence encodes a human CD3E, a human CD3D, and a human CD3G In some embodiments, the human CD3E comprises an amino acid sequence that is at least 70%, 75%, 80%, 85%, 90%, 95%, 96%, 97%, 98%, 99%, or 100% identical to SEQ ID NO: 2; the human CD3D comprises an amino acid sequence that is at least 70%, 75%, 80%, 85%, 90%, 95%, 96%, 97%, 98%, 99%, or 100% identical to SEQ ID NO: 4; and the human CD3G comprises an amino acid sequence that is at least 70%, 75%, 80%, 85%, 90%, 95%, 96%, 97%, 98%, 99%, or 100% identical to SEQ ID NO: 6. In some embodiments, the sequence encodes a chimeric CD3E, a human CD3D, and a human CD3G In some embodiments, the chimeric CD3E comprises an amino acid sequence that is at least 70%, 75%, 80%, 85%, 90%, 95%, 96%, 97%, 98%, 99%, or 100% identical to SEQ ID NO: 66; the human CD3D comprises an amino acid sequence that is at least 70%, 75%, 80%, 85%, 90%, 95%, 96%, 97%, 98%, 99%, or 100% identical to SEQ ID NO: 4; and the human CD3G comprises an amino acid sequence that is at least 70%, 75%, 80%, 85%, 90%, 95%, 96%, 97%, 98%, 99%, or 100% identical to SEQ ID NO: 6.

In one aspect, the disclosure is related to a method of making a genetically-modified non-human animal comprising: (a) replacing in at least one cell of the animal, at an endogenous CD3E gene locus, a sequence encoding a region of an endogenous CD3E with a sequence encoding a corresponding region of human CD3E; (b) replacing in at least one cell of the animal, at an endogenous CD3D gene locus, a sequence encoding a region of an endogenous CD3D with a sequence encoding a corresponding region of human CD3D; and/or (c) replacing in at least one cell of the animal, at an endogenous CD3G gene locus, a sequence encoding a region of an endogenous CD3G with a sequence encoding a corresponding region of human CD3G In some embodiments, step (b) and step (c) are performed at substantially the same time. In some embodiments, step (a) and step (b) are performed at substantially the same time. In some embodiments, step (a) and step (c) are performed at substantially the same time. In some embodiments, step (a), step (b) and step (c) are performed at substantially the same time. In some embodiments, step (a) is performed after step (b) and/or step (c). In some embodiments, step (a) is performed before step (b) and/or step (c). In some embodiments, step (b) is performed after step (a) and/or step (c). In some embodiments, step (c) is performed after step (a) and/or step (b). In some embodiments, step (c) is performed before step (a) and/or step (b).

In one aspect, the disclosure is related to a method of determining effectiveness of an anti-CD3E antibody, an anti-CD3D antibody, or an anti-CD3G antibody for the treatment of cancer, comprising: administering the anti-CD3E antibody, the anti-CD3D antibody, or the anti-CD3G antibody to the animal as described herein, in some embodiments, the animal has a tumor; and determining the inhibitory effects of the anti-CD3E antibody, the anti-CD3D antibody, or the anti-CD3G antibody to the tumor. In some embodiments, the tumor comprises one or more cancer cells that are injected into the animal. In some embodiments, determining the inhibitory effects of the anti-CD3E antibody, the anti-CD3D antibody, or the anti-CD3G antibody to the tumor involves measuring the tumor volume in the animal. In some embodiments, the tumor cells are melanoma cells, pancreatic carcinoma cells, mesothelioma cells, or solid tumor cells.

In one aspect, the disclosure is related to a method of determining effectiveness of an anti-CD3E antibody, an anti-CD3D antibody, or an anti-CD3G antibody in inhibiting an immune response, comprising: administering the anti-CD3E antibody, the anti-CD3D antibody, or the anti-CD3G antibody to the animal as described herein, in some embodiments, the animal has a tumor; and determining the effects of the anti-CD3E antibody, the anti-CD3D antibody, or the anti-CD3G antibody on the tumor. In some embodiments, the tumor comprises one or more cancer cells that are injected into the animal. In some embodiments, determining the effects of the anti-CD3E antibody, the anti-CD3D, or the anti-CD3G antibody on the tumor involves measuring the tumor volume in the animal.

In one aspect, the disclosure is related to a method of determining effectiveness of an anti-CD3E antibody, an anti-CD3D antibody, or an anti-CD3G antibody in inhibiting an immune response, comprising: administering the anti-CD3E antibody, the anti-CD3D antibody, or the anti-CD3G antibody to the animal as described herein, in some embodiments, the animal has a tumor; and determining the percentage of T cells in peripheral blood and/or tumor tissue.

In one aspect, the disclosure is related to a method of determining effectiveness of a bispecific antibody targeting CD3 and a tumor associated antigen (TAA) for the treatment of cancer, comprising: injecting cancer cells expressing the TAA to the animal as described herein; administering the bispecific antibody to the animal; and determining the inhibitory effects of the bispecific antibody to the tumor. In some embodiments, determining the inhibitory effects of the bispecific antibody to the tumor involves measuring the tumor volume in the animal. In some embodiments, the TAA is BCMA.

In one aspect, the disclosure is related to a protein comprising an amino acid sequence, in some embodiments, the amino acid sequence is one of the following:
(a) an amino acid sequence set forth in SEQ ID NO: 2, 4, 6, or 66;
(b) an amino acid sequence that is at least 90% identical to SEQ ID NO: 2, 4, 6, or 66;
(c) an amino acid sequence that is at least 91%, 92%, 93%, 94%, 95%, 96%, 97%, 98%, or 99% identical to SEQ ID NO: 2, 4, 6, or 66;
(d) an amino acid sequence that is different from the amino acid sequence set forth in SEQ ID NO: 2, 4, 6, or 66 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2, 4, 6, or 66.

In one aspect, the disclosure is related to a nucleic acid comprising a nucleotide sequence, In some embodiments, the nucleotide sequence is one of the following:
(a) a sequence that encodes the protein as described herein;
(b) SEQ ID NO: 7, 8, 9, 10, 11, 12, 13, 14, 15, 16, 17, 18, 35, 36, 63, 64, or 65;
(c) a sequence that is at least 90% identical to SEQ ID NO: 7, 8, 9, 10, 11, 12, 13, 14, 15, 16, 17, 18, 35, 36, 63, 64, or 65; and
(d) a sequence that is at least 91%, 92%, 93%, 94%, 95%, 96%, 97%, 98%, or 99% identical to SEQ ID NO: 7, 8, 9, 10, 11, 12, 13, 14, 15, 16, 17, 18, 35, 36, 63, 64, or 65.

In one aspect, the disclosure is related to a cell comprising the protein and/or the nucleic acid as described herein. In one aspect, the disclosure is related to an animal comprising the protein and/or the nucleic acid as described herein.

In one aspect, the disclosure relates to a genetically-modified, non-human animal whose genome comprises at least one chromosome comprising a sequence encoding a human or chimeric CD3E. In some embodiments, the sequence encoding the human or chimeric CD3E is operably linked to an endogenous regulatory element at the endogenous CD3E gene locus in the at least one chromosome.

In some embodiments, the sequence encoding a human or chimeric CD3E comprises a sequence encoding an amino acid sequence that is at least 50%, 55%, 65%, 70%, 75%, 80%, 85%, 90%, 95%, 99%, or 100% identical to human CD3E (NP_000724.1 (SEQ ID NO: 2)). In some embodiments, the sequence encoding a human or chimeric CD3E comprises a sequence encoding an amino acid sequence that is at least 50%, 55%, 65%, 70%, 75%, 80%, 85%, 90%, 95%, 99%, or 100% identical to SEQ ID NO: 66. In some embodiments, the sequence encoding a human or chimeric CD3E comprises a sequence that is at least 50%, 55%, 65%, 70%, 75%, 80%, 85%, 90%, 95%, 99%, or 100% identical to amino acids 1-126 of SEQ ID NO: 2.

In some embodiments, the animal is a mammal, e.g., a monkey, a rodent or a mouse. In some embodiments, the animal is a mouse.

In some embodiments, the animal does not express endogenous CD3E. In some embodiments, the animal has one or more cells expressing human or chimeric CD3E.

In some embodiments, the animal has one or more cells expressing human or chimeric CD3E, and the expressed human or chimeric CD3E can form a CD3 complex with human CD3 gamma (γ), delta (δ) and zeta (ζ) polypeptides. In some embodiments, the animal has one or more cells expressing human or chimeric CD3E, and the expressed human or chimeric CD3E can form a CD3 complex with endogenous CD3 gamma (γ), delta (δ) and zeta (ζ) polypeptides.

In one aspect, the disclosure relates to a genetically-modified, non-human animal, wherein the genome of the animal comprises a replacement of a sequence encoding a region of endogenous CD3E with a sequence encoding a corresponding region of human CD3E at an endogenous CD3E gene locus.

In some embodiments, the sequence encoding the corresponding region of human CD3E is operably linked to an endogenous regulatory element at the endogenous CD3E locus, and one or more cells of the animal expresses a chimeric CD3E. In some embodiments, the animal does not express endogenous CD3E. In some embodiments, the replaced locus is the extracellular region of CD3E.

In some embodiments, the animal has one or more cells expressing a chimeric CD3E having an extracellular region, a transmembrane region, and a cytoplasmic region

In some embodiments, the extracellular region comprises a sequence that is at least 50%, 60%, 70%, 80%, 90%, 95%, or 99% identical to the extracellular region of human CD3E.
In some embodiments, the extracellular region of the chimeric CD3E has a sequence that has at least 10, 20, 30, 40, 50, 60, 70, 80, 90, 100, 110, 120, 130, 140, 150, 160, 170, or 180 contiguous amino acids that are identical to a contiguous sequence present in the extracellular region of human CD3E.

In some embodiments, the animal is a mouse, and the sequence encoding the region of endogenous CD3E is exon 2, exon 3, exon 4, exon 5, and/or exon 6 of the endogenous mouse CD3E gene. In some embodiments, the sequence encoding the region of endogenous CD3E is exon 2, exon 3, exon 4, exon 5, exon 6, exon 7, and/or exon 8 of the endogenous mouse CD3E gene.

In some embodiments, the animal is heterozygous with respect to the replacement at the endogenous CD3E gene locus. In some embodiments, the animal is homozygous with respect to the replacement at the endogenous CD3E gene locus.

In one aspect, the disclosure relates to methods for making a genetically-modified, non-human animal. The methods involve replacing in at least one cell of the animal, at an endogenous CD3E gene locus, a sequence encoding a region of an endogenous CD3E with a sequence encoding a corresponding region of human CD3E.

In some embodiments, the sequence encoding the corresponding region of human CD3E comprises exon 2, exon 3, exon 4, exon 5, exon 6, exon 7, exon 8, and/or exon 9, or a part thereof, of a human CD3E gene. In some embodiments, the sequence encoding the corresponding region of CD3E comprises exon 2, exon 3, exon 4, exon 5, exon 6, and/or exon 7, or a part thereof, of a human CD3E gene. In some embodiments, the sequence encoding the corresponding region of human CD3E encodes amino acids 1-126 of SEQ ID NO: 2.

In some embodiments, the region is located within the extracellular region of CD3E.

In some embodiments, the animal is a mouse, and the endogenous CD3E locus is exon 2, exon 3, exon 4, exon 5, and/or exon 6 of the mouse CD3E gene. In some embodiments, the endogenous CD3E locus is exon 2, exon 3, exon 4, exon 5, exon 6, exon 7, and/or exon 8 of the mouse CD3E gene.

In one aspect, the disclosure relates to a non-human animal comprising at least one cell comprising a nucleotide sequence encoding a genetically engineered CD3E polypeptide. In some embodiments, the genetically engineered CD3E polypeptide comprises at least 50 contiguous amino acid residues that are identical to the corresponding contiguous amino acid sequence of a human CD3E. In some embodiments, the animal expresses the genetically engineered CD3E.

In some embodiments, the genetically engineered CD3E polypeptide has at least 50 contiguous amino acid residues that are identical to amino acid sequence of a human CD3E extracellular region.

In some embodiments, the genetically engineered CD3E polypeptide comprises a sequence that is at least 50%, 60%, 70%, 80%, 90%, 95%, or 99% identical to amino acids 1-126 of SEQ ID NO: 2. In some embodiments, the genetically engineered CD3E polypeptide comprises a sequence that is at least 50%, 60%, 70%, 80%, 90%, 95%, or 99% identical to SEQ ID NO: 2.

In some embodiments, the nucleotide sequence is operably linked to an endogenous CD3E regulatory element of the animal.

In some embodiments, the genetically engineered CD3E polypeptide comprises an endogenous CD3E transmembrane region and/or an endogenous CD3E cytoplasmic region.

In some embodiments, the nucleotide sequence is integrated to an endogenous CD3E gene locus of the animal.

In some embodiments, the genetically engineered CD3E has at least one mouse CD3E activity and/or at least one human CD3E activity.

In one aspect, the disclosure relates to methods of making a genetically-modified mouse cell that expresses a human or chimeric CD3E. The methods involve replacing at an endogenous mouse CD3E gene locus, a nucleotide sequence encoding a region of mouse CD3E with a nucleotide sequence encoding a corresponding region of human CD3E, thereby generating a genetically-modified mouse cell that includes a nucleotide sequence that encodes the human or chimeric CD3E. In some embodiments, the mouse cell expresses the human or chimeric CD3E.

In some embodiments, the chimeric CD3E comprises an extracellular region of human CD3E comprising a human signal peptide sequence; and a transmembrane and/or a cytoplasmic region of mouse CD3E.

In some embodiments, the nucleotide sequence encoding the human or chimeric CD3E is operably linked to an endogenous CD3E regulatory region, e.g., promoter.

In some embodiments, the animal further comprises a sequence encoding an additional human or chimeric protein. In some embodiments, the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), TNF Receptor Superfamily Member 4 (OX40), CD3E, CD3γ, CD40, or CD278.

In one aspect, the disclosure relates to methods of determining effectiveness of an anti-CD3E antibody for the treatment of cancer. The methods involve administering the anti-CD3E antibody to the animal as described herein, wherein the animal has a tumor; and determining the inhibitory effects of the anti-CD3E antibody to the tumor. In some embodiments, the tumor comprises one or more cancer cells that are injected into the animal. In some embodiments, determining the inhibitory effects of the anti-CD3E antibody to the tumor involves measuring the tumor volume in the animal. In some embodiments, the tumor cells are melanoma cells, pancreatic carcinoma cells, mesothelioma cells, or solid tumor cells.

In one aspect, the disclosure relates to methods of determining effectiveness of an anti-CD3E antibody in inhibiting an immune response. The methods involve administering the anti-CD3E antibody to the animal as described herein, wherein the animal has a tumor; and determining the effects of the anti-CD3E antibody on the tumor. In some embodiments, the tumor comprises one or more cancer cells that are injected into the animal. In some embodiments, determining the effects of the anti-CD3E antibody on the tumor involves measuring the tumor volume in the animal.

In one aspect, the disclosure relates to methods of determining effectiveness of an anti-CD3E antibody in inhibiting an immune response. The methods involve administering the anti-CD3E antibody to the animal as described herein; and determining the percentage of T cells in peripheral blood.

In one aspect, the disclosure relates to methods of determining effectiveness of an anti-CD3E antibody in activating T cells. The methods involve administering the anti-CD3E antibody to the animal as described herein; and determining the percentage of CD25+ T cells or CD 69+ T cells in a cell population. In some embodiments, the cell population is derived from the spleen, thymus, inguinal lymph nodes or mesenteric lymph nodes of the animal.

In one aspect, the disclosure relates to methods of determining effectiveness of an anti-CD3E antibody in activating T cells. The methods involve administering the anti-CD3E antibody to the animal as described herein; and determining the expression of CD3 in a cell population. In some embodiments, the cell population is derived from the spleen, thymus, inguinal lymph nodes or mesenteric lymph nodes of the animal.

In one aspect, the disclosure relates to methods of determining effectiveness of an anti-CD3 (e.g., CD3E, CD3D, or CD3G) antibody and an additional therapeutic agent for the treatment of a tumor. The methods involve administering the anti-CD3 antibody and the additional therapeutic agent to the animal as described herein, wherein the animal has a tumor; and determining the inhibitory effects on the tumor.

In some embodiments, the animal further comprises a sequence encoding a human or chimeric programmed cell death protein 1 (PD-1). In some embodiments, the animal further comprises a sequence encoding a human or chimeric programmed death-ligand 1 (PD-L1). In some embodiments, the additional therapeutic agent is an anti-PD-1 antibody or an anti-PD-L1 antibody. In some embodiments, the tumor comprises one or more tumor cells that express PD-L1 or PD-L2.

In some embodiments, the tumor is caused by injection of one or more cancer cells into the animal. In some embodiments, determining the inhibitory effects of the treatment involves measuring the tumor volume in the animal.

In some embodiments, the animal has melanoma, pancreatic carcinoma, mesothelioma, hematological malignancies (e.g., Non-Hodgkin's lymphoma, lymphoma, chronic lymphocytic leukemia), or solid tumors.

In one aspect, the disclosure relates to methods of determining effectiveness of an anti-CD3 antibody (e.g., an anti-CD3E antibody, an anti-CD3D antibody, or an anti-CD3G antibody) for the treatment of various immune-related disorders, e.g., autoimmune diseases.

In another aspect, the disclosure also provides a genetically-modified, non-human animal whose genome comprise a disruption in the animal's endogenous CD3E gene, wherein the disruption of the endogenous CD3E gene comprises deletion of exon 1, exon 2, exon 3, exon 4, exon 5, exon 6, exon 7, and/or exon 8 or part thereof of the endogenous CD3E gene.

In some embodiments, the disruption of the endogenous CD3E gene comprises deletion of one or more exons or part of exons selected from the group consisting of exon 1, exon 2, exon 3, exon 4, exon 5, exon 6, exon 7, and exon 8 of the endogenous CD3E gene.

In some embodiments, the disruption of the endogenous CD3E gene further comprises deletion of one or more introns or part of introns selected from the group consisting of intron 1, intron 2, intron 3, intron 4, intron 5, intron 6, and intron 7 of the endogenous CD3E gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous CD3E gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, and/or exon 8 (e.g., deletion of at least 50 nucleotides of exon 2 or exon 7).

The disclosure also relates to a cell including the targeting vector as described herein.

The disclosure also relates to non-human mammal generated through the methods as described herein.

In some embodiments, the genome thereof contains human gene(s).

In some embodiments, the non-human mammal is a rodent. In some embodiments, the non-human mammal is a mouse.

In some embodiments, the non-human mammal expresses a protein encoded by a humanized CD3E gene, a humanized CD3D gene, and/or a humanized CD3G gene.

The disclosure also relates to an offspring of the non-human mammal.

In another aspect, the disclosure relates to a tumor bearing non-human mammal model, characterized in that the non-human mammal model is obtained through the methods as described herein.

In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to CD3E, CD3D, and/or CD3G genomic DNA sequences of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the CD3E, CD3D, and CD3G gene function; anti-human CD3E, CD3D, and/or CD3G antibodies; drugs for human CD3E, CD3D, and/or CD3G targeting sites; the drugs or efficacies for human CD3E, CD3D, and/or CD3G targeting sites; and the drugs for immune-related diseases and antitumor drugs.

As used herein, the term “nucleotide” refers to native or modified ribonucleotide sequences or deoxyribonucleotide sequences (e.g., DNA, cDNA, pre-mRNA, mRNA, rRNA, hnRNA, miRNAs, scRNA, snRNA, siRNA, sgRNA, or tRNA)

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing mouse and human CD3E gene loci.

FIG. 2 is a schematic diagram showing mouse and human CD3D gene loci.

FIG. 3 is a schematic diagram showing mouse and human CD3G gene loci.

FIG. 4 is a schematic diagram showing humanized CD3E, CD3D, and CD3G gene loci.

FIG. 5 is a schematic diagram showing a CD3E, CD3D, and CD3G gene targeting strategy. mCD3EDG represents wild-type mouse CD3E, CD3D and CD3G loci; chiCD3EDG-1 represents genetically-modified mouse CD3D and CD3G loci; and chiCD3EDG-2 represents genetically-modified mouse CD3E, CD3D and CD3G loci. V1 is targeting vector 1, and V2 is targeting vector 2.

FIG. 6 shows Southern Blot results of cells after CD3DG gene recombination using the CD3DG-5′ Probe, HygR Probe, and CD3DG-3′ Probe. 3-F01, 3-H02, 3-H09, 4-A08, 4-009, 4-E05, 4-G11, and 4-H03 are clone numbers. WT is a wild-type control.

FIG. 7 shows Southern Blot results of cells after CD3E gene recombination using the CD3E-5′ Probe, CD3E-3′ Probe, and Neo Probe. A-A01, 1-A09, 1-H06, 1-G03, 1-G04, 2-F10, 2-H03, 3-E05, 3-E07, 3-G05, 4-F10, 4-G11, and 1-D10 are clone numbers. WT is a wild-type control.

FIGS. 8A-8D show PCR identification results of F1 generation mice by primers CD3DG-WT-F and CD3DG-WT-R (FIG. 8A); CD3DG-WT-F and CD3DG-Mut-R (FIG. 8B); CD3E-WT-F and CD3E-WT-R (FIG. 8C); and CD3E-WT-F and CD3E-Mut-R (FIG. 8D). F1-01, F1-02, and F1-03 are mouse numbers. M is a marker. PC is a positive control. WT is a wild-type control. H₂O is a water control.

FIGS. 9A-9G show RT-PCR detection results of mouse CD3E, human CD3E, mouse CD3D, human CD3D, mouse CD3G human CD3G and GAPDH, respectively, in C57BL/6 wild-type mouse (+1+) and humanized CD3EDG homozygous mouse (H/H). GAPDH was used as an internal control. H₂O is a water control.

FIG. 10A is a picture of isolated spleens from three C57BL/6 wild-type mice and three humanized CD3EDG homozygous mice (H/H).

FIG. 10B shows the average spleen weight of three C57BL/6 wild-type mice and three humanized CD3EDG homozygous mice (H/H).

FIG. 10C shows the average number of splenocytes in three C57BL/6 wild-type mice and three humanized CD3EDG homozygous mice (H/H).

FIG. 11A is a picture of isolated thymuses from three C57BL/6 wild-type mice and three humanized CD3EDG homozygous mice (H/H).

FIG. 11B shows the average thymus weight of three C57BL/6 wild-type mice and three humanized CD3EDG homozygous mice (H/H).

FIG. 11C shows the average number of thymocytes in three C57BL/6 wild-type mice and three humanized CD3EDG homozygous mice (H/H).

FIG. 12 shows the percentage of leukocyte subtypes in the thymus of C57BL/6 wild-type mice and humanized CD3EDG homozygous mice (H/H) as determined by flow cytometry. DN is a double-negative control. DP is a double-positive control.

FIG. 13 shows the percentage of T cell subtypes in the thymus of C57BL/6 wild-type mice and humanized CD3EDG homozygous mice (H/H) as determined by flow cytometry. DN is a double-negative control. DP is a double-positive control.

FIG. 14 shows the percentage of leukocyte subtypes in the spleen of C57BL/6 wild-type mice and humanized CD3EDG homozygous mice (H/H) as determined by flow cytometry. B stands for B cells; T stands for T cells, NK stands for NK cells, Gran stands for granulocytes, DC stands for dendritic cells, Mo stands for microphages, and Mon stands for monocytes.

FIG. 15 shows the percentage of T cell subtypes in the spleen of C57BL/6 wild-type mice and humanized CD3EDG homozygous mice (H/H) as determined by flow cytometry.

FIG. 16 shows the percentage of leukocyte subtypes in lymph nodes of C57BL/6 wild-type mice and humanized CD3EDG homozygous mice (H/H) as determined by flow cytometry.

FIG. 17 shows the percentage of T cell subtypes in in lymph nodes of C57BL/6 wild-type mice and humanized CD3EDG homozygous mice (H/H) as determined by flow cytometry.

FIG. 18A shows CD4+ T cell proliferation results of C57BL/6 wild-type mice as determined by flow cytometry. The CD4+ T cells from mouse spleen tissues were treated with PBS, an anti-mouse CD3E antibody (anti-mCD3E), an anti-human CD3E antibody (anti-hCD3E), anti-mCD3E in combination with an anti-mouse CD28 antibody (anti-mCD3E/ant-mCD28), or anti-hCD3E in combination with anti-mCD28 (anti-hCD3E/anti-mCD28).

FIG. 18B shows CD4+ T cell proliferation results of humanized CD3EDG homozygous mice as determined by flow cytometry. The CD4+ T cells from mouse spleen tissues were treated with PBS, anti-mCD3E, anti-hCD3E, anti-mCD3E/ant-mCD28, or anti-hCD3E/anti-mCD28.

FIG. 19A shows CD8+ T cell proliferation results of C57BL/6 wild-type mice as determined by flow cytometry. The CD8+ T cells from mouse spleen tissues were treated with PBS, anti-mCD3E, anti-hCD3E, anti-mCD3E/ant-mCD28, or anti-hCD3E/anti-mCD28.

FIG. 19B shows CD8+ T cell proliferation results of humanized CD3EDG homozygous mice as determined by flow cytometry. The CD8+ T cells from mouse spleen tissues were treated with PBS, anti-mCD3E, anti-hCD3E, anti-mCD3E/ant-mCD28, or anti-hCD3E/anti-mCD28.

FIGS. 20A-20B show expression levels of IL2 and IFN-γ in C57BL/6 wild-type mice and humanized CD3EDG homozygous mice (H/H) after treatment of PBS, anti-mCD3E (mCD3), anti-hCD3E (hCD3), anti-mCD3E/anti-mCD28 (mCD3+mCD28), anti-hCD3E/anti-mCD28 (hCD3+mCD28), or medium for 24 hours.

FIGS. 20C-20D show expression levels of IL2 and IFN-γ in C57BL/6 wild-type mice and humanized CD3EDG homozygous mice (H/H) after treatment of PBS, anti-mCD3E (mCD3), anti-hCD3E (hCD3), anti-mCD3E/anti-mCD28 (mCD3+mCD28), anti-hCD3E/anti-mCD28 (hCD3+mCD28), or medium for 48 hours.

FIGS. 20E-20F show expression levels of IL2 and IFN-γ in C57BL/6 wild-type mice and humanized CD3EDG homozygous mice (H/H) after treatment of PBS, anti-mCD3E (mCD3), anti-hCD3E (hCD3), anti-mCD3E/anti-mCD28 (mCD3+mCD28), anti-hCD3E/anti-mCD28 (hCD3+mCD28), or medium for 72 hours.

FIG. 21A shows the mouse serum concentrations of total IgGs IgG1, IgG2b, IgG2c, IgG3, IgM, and IgA in C57BL/6 wild-type mice and humanized CD3EDG mice before immunization.

FIG. 21B shows ELISA results of serum OVA-specific antibody titers in C57BL/6 wild-type mice and humanized CD3EDG mice after the second and third immunizations.

FIG. 22A shows the tumor volume of the different groups of humanized CD3EDG homozygous mice that were injected with mouse colon cancer cells MC38, and were treated with an anti-human IgG antibody hIgG Ab (G1), an anti-mouse PD-1 antibody mPD-1 Ab (G2), or an anti-human CD3E antibody hCD3E Ab (G3).

FIG. 22B shows the average body weight of the different groups of humanized CD3EDG homozygous mice that were injected with mouse colon cancer cells MC38, and were treated with an anti-human IgG antibody hIgG Ab (G1), an anti-mouse PD-1 antibody mPD-1 Ab (G2), or an anti-human CD3E antibody hCD3E Ab (G3).

FIG. 23A shows the average T cell number in peripheral blood of humanized CD3EDG homozygous mice that were treated with hIgG Ab (G1), mPD-1 Ab (G2), or hCD3E Ab (G3).

FIG. 23B shows the average B cells number in peripheral blood of humanized CD3EDG homozygous mice that were treated with hIgG Ab (G1), mPD-1 Ab (G2), or hCD3E Ab (G3).

FIG. 24A shows the average T cell number in tumor tissue of humanized CD3EDG homozygous mice that were treated with hIgG Ab (G1), mPD-1 Ab (G2), or hCD3E Ab (G3).

FIG. 24B shows the average B cells number in tumor tissue of humanized CD3EDG homozygous mice that were treated with hIgG Ab (G1), mPD-1 Ab (G2), or hCD3E Ab (G3).

FIG. 25 is a schematic diagram showing a CD3E, CD3D, and CD3G gene targeting strategy. mCD3EDG represents wild-type mouse CD3E, CD3D and CD3G loci; chiCD3EDG-1 represents genetically-modified mouse CD3D and CD3G loci; and chiCD3EDG-3 represents genetically-modified mouse CD3E, CD3D and CD3G loci. V1 is targeting vector 1, and V3 is targeting vector 3.

FIG. 26 shows the cytotoxicity-concentration curves of in vitro killing assays. Splenocytes from humanized CD3EDG mice (effector cells) and MC38 cells expressing human BCMA protein (target cells) were incubated at a 20:1 or 40:1 ratio for 48 hours in the present of an anti-human CD3/BCMA bispecific antibody.

FIG. 27A shows the tumor volume of the different groups of humanized CD3EDG homozygous mice that were injected with transfected MC3 8 expressing human BCMA protein, and were treated with physiological saline (PS; G1), or an anti-human CD3/BCMA bispecific antibody Abl at different doses (G2, G3, and G4).

FIG. 27B shows the average body weight of the different groups of humanized CD3EDG homozygous mice that were injected with transfected MC3 8 expressing human BCMA protein, and were treated with physiological saline (PS; G1), or an anti-human CD3/BCMA bispecific antibody Abl at different doses (G2, G3, and G4).

FIG. 28 shows the alignment between mouse CD3E amino acid sequence (NP_031674.1; SEQ ID NO: 1) and human CD3E amino acid sequence (NP_000724.1; SEQ ID NO: 2).

FIG. 29 shows the alignment between mouse CD3D amino acid sequence (NP_038515.3; SEQ ID NO: 3) and human CD3D amino acid sequence (NP_000723.1; SEQ ID NO: 4).

FIG. 30 shows the alignment between mouse CD3G amino acid sequence (NP_033980.1; SEQ ID NO: 5) and human CD3G amino acid sequence (NP_000064.1; SEQ ID NO: 6).

### DETAILED DESCRIPTION

Autoimmune processes are related to defects in immunologic tolerance, a state of immune system unresponsiveness to an antigen. Tolerance is maintained by multiple mechanisms including deletion, anergy, and active cellular regulation and strategies to induce immune tolerance are being developed for the treatment of autoimmunity.

CD3 (cluster of differentiation 3) is a T cell co-receptor that is involved in activating both the cytotoxic T cell (CD8+ naive T cells) and also T helper cells (CD4+ naive T cells). Depending on the conditions used, antibodies against CD3 can either stimulate T cells to divide or inhibit the development of effector functions such as cytotoxicity. Anti-CD3 antibody therapy has a demonstrated potential in the context of treating autoimmune diseases. However, the efficacy of anti-CD3 therapy has been limited by in vivo toxicities. A well-known anti-CD3 antibody, OKT3, is used routinely in clinical therapy of transplant rejection but is known to mediate dramatic cytokine release in vivo, leading to a “flu-like” syndrome. This effect has been identified with a humoral response against the OKT3 molecule as well as a release of proinflammatory cytokines such as TNF-α. These physiological toxicities restrict the dosage regimens available to patients with anti-CD3 therapy and limit the overall efficacy of anti-CD3 treatment of autoimmune disease.

Experimental animal models are an indispensable research tool for studying the effects of these CD3 targeting therapies (e.g., anti-CD3E antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U.S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wuetal. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.

Y., 1986); each of which is incorporated herein by reference in its entirety.

### CD3

Cluster of differentiation 3 (CD3) is a multimeric protein complex, known historically as the T3 complex, and is composed of four distinct polypeptide chains; epsilon (ε) (CD3E), gamma (γ) (CD3G), delta (δ) (CD3D) and zeta (ζ) (CD3Z), that assemble and function as three pairs of dimers (εγ, εδ, ζζ). The CD3 complex serves as a T cell co-receptor that associates non-covalently with the T cell receptor (TCR). The CD3 protein complex is a defining feature of the T cell lineage, therefore anti-CD3 antibodies can be used effectively as T cell markers.

Ligation of the TCR/CD3 results in activation of src and syk family PTKs associated with the intracellular CD3 domains that then activate PLC and Ras-dependent pathways. However, signaling via the TCR complex is not a linear event starting at the receptor and ending in the nucleus. Instead, there appears to be complex feedback and feedforward regulation at each step.

Because CD3 is required for T cell activation, drugs (often monoclonal antibodies) that target it are being investigated as immunosuppressant therapies (e.g., otelixizumab) for graft vs host disease, and various autoimmune diseases (e.g., arthritis, type 1 diabetes).

CD3E (or CD3E) is a non-glycosylated polypeptide chain of 20 kDa. The existence of an epitope on the ε polypeptide that is conserved among many species has made it as a preferable target for antibodies that target CD3. Therapeutic anti-CD3E antibodies bind to the epsilon chain of the CD3/TCR complex that characterizes T lymphocytes. Several nonmutually exclusive mechanisms have been proposed to explain the therapeutic effect of anti-CD3E antibodies. After a short lasting capping of the CD3 complex, the CD3/T-cell receptor complex disappears from the cell surface by internalization or shedding, a process called antigenic modulation that renders T cells temporarily blind to their cognate antigens. Anti-CD3E antibody-induced signaling can also preferentially induce anergy or apoptosis in activated T cells while sparing Tregs. Heterogeneity of TCR expression by different T-cell subsets might explain the differential effect of anti-CD3E antibody on effector versus regulatory or naïve T cells.

The tolerogenic function of anti-CD3E antibody is independent of effector functions that are linked to the Fc region of the antibody, such as complement-dependent cytotoxicity (CDC), antibody-dependent cellular cytotoxicity (ADCC) and antibody-dependent cell phagocytosis (ADCP), as F(ab′)2 fragments are sufficient for tolerance induction. It has been shown that T cells become rapidly activated in response to intravenous anti-CD3E antibody as measured by increased expression of CD69 and CD25 and serum concentrations of TGF-β and IFN-γ briefly after injection, even when using nonmitogenic anti-CD3E antibody. The direct effects of anti-CD3E antibody on T cells (capping, antigenic modulation, induction of apoptosis and anergy) are all short-term and are gone after clearance of the antibody from the circulation. Yet, the pharmacological effects mediated by anti-CD3E antibody therapy can be long lasting, indicating that additional and more durable mechanisms are involved in anti-CD3E antibody mediated tolerance. Evidence suggests a link between anti-CD3E antibody-induced apoptosis, phagocytosis of the resulting apoptotic bodies by macrophages and a subsequent increase of TGF-β. TGF-β plays an essential role in regulating immune responses and the production of TGF-β is crucial for the therapeutic effect of anti-CD3E antibody. TGF-β has pleiotropic effects on the adaptive immunity, including induction of adaptive FoxP3+ Tregs, inhibition of T-cell activation and proliferation and blocking dendritic cell maturation, and all these outcomes are observed after anti-CD3E antibody mediated tolerance induction. Indeed, it has been demonstrated that anti-CD3E antibody therapy increases TGF-β dependent Tregs, renders effector T cells more susceptible to TGF-β mediated regulation and confers a tolerogenic phenotype to dendritic cells.

A detailed description of CD3, and the use of anti-CD3E antibodies to treat various diseases are described, e.g., in Smith-Garvin, et al. “T cell activation.” Annual review of immunology 27 (2009): 591-619; Kuhn, et al. “Therapeutic anti-CD3 monoclonal antibodies: from bench to bedside.” Immunotherapy 8.8 (2016): 889-906; US 20060275292; and US 20070292416; each of which is incorporated by reference in its entirety.

### CD3E

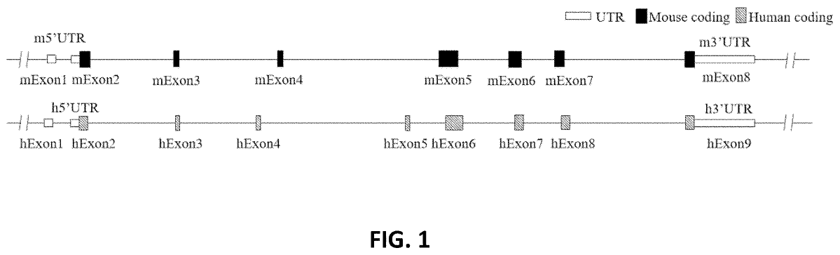

In human genomes, CD3E gene (Gene ID: 916) locus has nine exons, exon 1, exon 2, exon 3, exon 4, exon 5, exon 6, exon 7, exon 8, and exon 9 (FIG. 1). The CD3E protein also has an extracellular region, a transmembrane region, and a cytoplasmic region, and the signal peptide is located at the extracellular region of CD3E. The nucleotide sequence for human CD3E mRNA is NM_000733.4, and the amino acid sequence for human CD3E is NP_000724.1 (SEQ ID NO: 2). The location for each exon and each region in human CD3E nucleotide sequence and amino acid sequence is listed below:

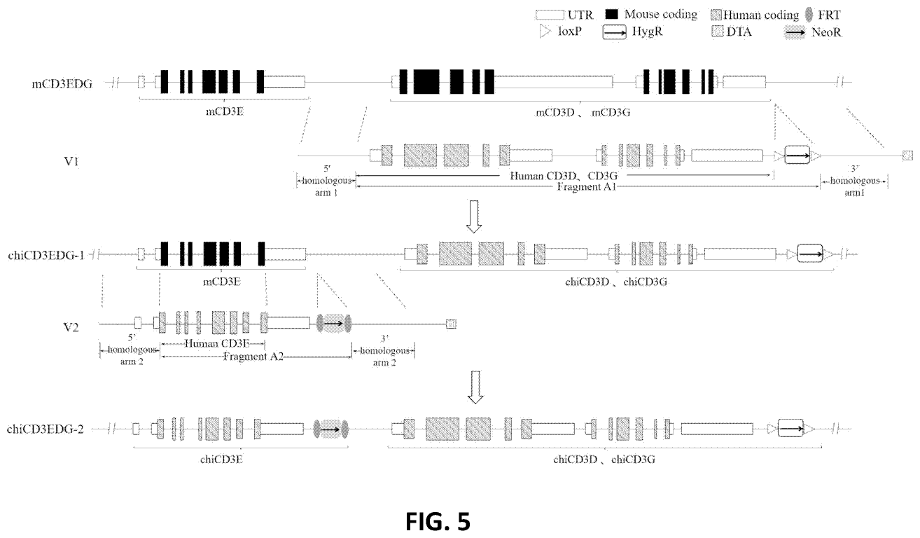

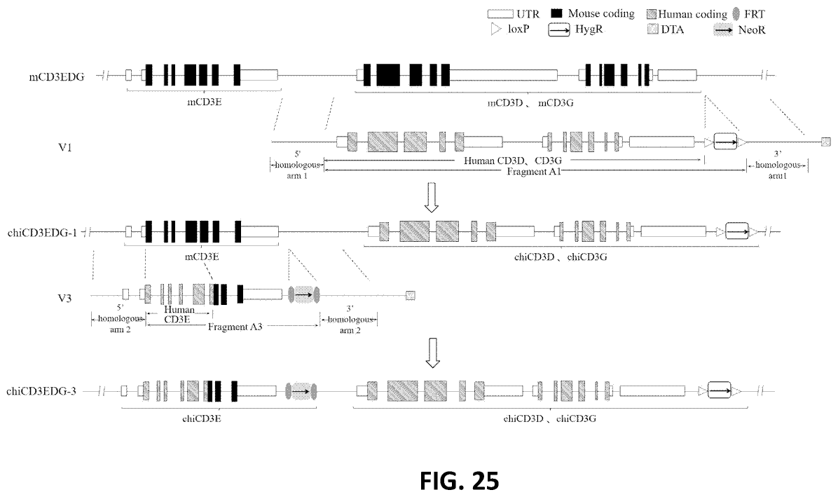

TABLE 1

| human CD3E | | NP_000724.1 (207 aa) |
|---|---|---|
| (approximate location) | NM_000733.4 | SEQ ID NO: 2 |
| Exon 1 | 1-47 | Non coding |
| Exon 2 | 48-155 | 1-16 |
| Exon 3 | 156-176 | 17-23 |
| Exon 4 | 177-191 | 24-28 |
| Exon 5 | 192-209 | 29-34 |
| Exon 6 | 210-458 | 35-117 |
| Exon 7 | 459-626 | 118-173 |
| Exon 8 | 627-673 | 174-189 |
| Exon 9 | 674-1361 | 190-207 |
| Signal peptide | 107-172 | 1-22 |
| Extracellular | 173-484 | 23-126 |
| Transmembrane | 485-562 | 127-152 |
| Cytoplasmic | 563-727 | 153-207 |
| Donor region in FIG. 5 | 107-730 | 1-207 + TGA |
| Donor region in FIG. 25 | 107-484 | 1-126 |

The human CD3E gene (Gene ID: 916) is located in Chromosome 11 of the human genome, which is located from 118304730 to 118316173 of NC_000011.10 (GRCh38.p13 (GCF_000001405.39)). The 5′-UTR is from 118,304,730 to 118,304,776 and 118,304,894 to 118,304,952, exon 1 is from 118,304,730 to 118,304,776, the first intron is from 118,304,777 to 118,304,893, exon 2 is from 118,304,894 to 118,305,001, the second intron is from 118,305,002 to 118,307,287, exon 3 is from 118,307,288 to 118,307,308, the third intron is from 118,307,309 to 118,308,426, exon 4 is from 118,308,427 to 118,308,441, the fourth intron is from 118,308,442 to 118,312,152, exon 5 is from 118,312,153 to 118,312,170, the fifth intron is from 118,312,171 to 118,312,617, exon 6 is from 118,312,618 to 118,312,866, the sixth intron is from 118,312,867 to 118,313,706, exon 7 is from 118,313,707 to 118,313,874, the seventh intron is from 118,313,875 to 118,314,447, exon 8 is from 118,314,448 to 118,314,494, the eighth intron is from 118,314,495 to 118,315,485, exon 9 is from 118,315,486 to 118,316,173, the 3′-UTR is from 118,315,543 to 118,316,173, based on transcript NM_000733.4. All relevant information for human CD3E locus can be found in the NCBI website with Gene ID: 916, which is incorporated by reference herein in its entirety.

In mice, CD3E gene locus has eight exons, exon 1, exon 2, exon 3, exon 4, exon 5, exon 6, exon 7, and exon 8 (FIG. 1). The mouse CD3E protein also has an extracellular region, a transmembrane region, and a cytoplasmic region, and the signal peptide is located at the extracellular region of CD3E. The nucleotide sequence for mouse CD3E mRNA is NM_007648.5, the amino acid sequence for mouse CD3E is NP_031674.1 (SEQ ID NO: 1). The location for each exon and each region in the mouse CD3E nucleotide sequence and amino acid sequence is listed below:

TABLE 2

| Mouse CD3E | | NP_031674.1 (189 aa) |
|---|---|---|
| (approximate location) | NM_007648.5 | SEQ ID NO: 1 |
| Exon 1 | 1-46 | Non coding |
| Exon 2 | 47-148 | 1-16 |
| Exon 3 | 149-166 | 17-22 |
| Exon 4 | 167-181 | 23-27 |
| Exon 5 | 182-397 | 28-99 |
| Exon 6 | 398-565 | 100-155 |
| Exon 7 | 566-612 | 156-171 |
| Exon 8 | 613-1457 | 172-189 |
| Signal peptide | 100-165 | 1-22 |
| Extracellular | 166-423 | 23-108 |
| Transmembrane | 424-501 | 109-134 |
| Cytoplasmic | 502-666 | 135-189 |
| Replaced region in FIG. 5 | 100-669 | 1-189 + TGA |
| Replaced region in FIG. 25 | 100-423 | 1-108 |

The mouse CD3E gene (Gene ID: 12501) is located in Chromosome 9 of the mouse genome, which is located from 44910033 to 44920961 of NC_000075.7 (GRCm39 (GCF_000001635.27)). The 5′-UTR is from 45,009,613 to U.S. Pat. Nos. 45,009,566 and 45,009,449 to 45,009,397, exon 1 is from 45,009,613 to 45,009,566, the first intron is from 45,009,565 to 45,009,450, exon 2 is from 45,009,449 to 45,009,348, the second intron is from 45,009,347 to 45,007,194, exon 3 is from 45,007,193 to 45,007,176, the third intron is from 45,007,175 to 45,005,527, exon 4 is from 45,005,526 to 45,005,512, the fourth intron is from 45,005,511 to 45,002,354, exon 5 is from 45,002,353 to 45,002,138, the fifth intron is from 45,002,137 to 45,001,147, exon 6 is from 45,001,146 to 45,000,979, the sixth intron is from 45,000,978 to 45,000,482, exon 7 is from 45,000,481 to 45,000,435, the seventh intron is from 45,000,434 to 44,999,588, exon 8 is from 44,999,587 to 44,998,740, the 3′-UTR is from 44,999,530 to 44,998,740, based on transcript NM_007648.5. All relevant information for mouse CD3E locus can be found in the NCBI website with Gene ID: 12501, which is incorporated by reference herein in its entirety.

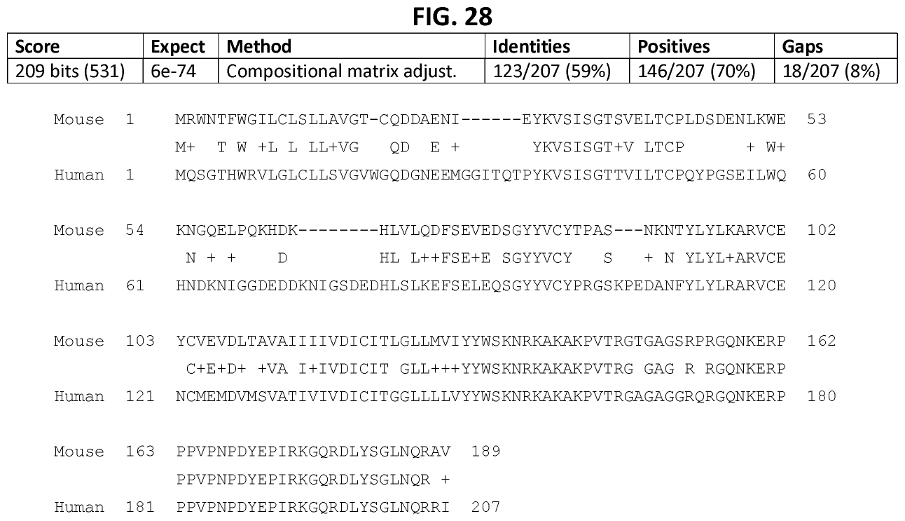

FIG. 28 shows the alignment between mouse CD3E amino acid sequence (NP_031674.1; SEQ ID NO: 1) and human CD3E amino acid sequence (NP_000724.1; SEQ ID NO: 2). Thus, the corresponding amino acid residue or region between human and mouse CD3E can be found in FIG. 28.

CD3E genes, proteins, and locus of the other species are also known in the art. For example, the gene ID for CD3E in Rattus norvegicus is 315609, the gene ID for CD3E in Macaca mulatta (Rhesus monkey) is 699467, the gene ID for CD3E in Canis lupus familiaris (dog) is 442981, and the gene ID for CD3E in Sus scrofa (pig) is 397455. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) CD3E nucleotide sequence and/or amino acid sequences. In some embodiments, the entire sequence of mouse exon 1, exon 2, exon 3, exon 4, exon 5, exon 6, exon 7, exon 8, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, and/or exon 8 (e.g., exon 2, exon 3, exon 4, exon 5, exon 6, exon 7, and exon 8) are replaced by the human exon 1, exon 2, exon 3, exon 4, exon 5, exon 6, exon 7, exon 8, and/or exon 9 (e.g., exon 2, exon 3, exon 4, exon 5, exon 6, exon 7, exon 8, and exon 9) sequence.

In some embodiments, the genetically-modified non-human animal described herein comprises a sequence encoding a human or humanized CD3E protein. In some embodiments, the humanized CD3E protein comprises an endogenous cytoplasmic region. In some embodiments, the humanized CD3E protein comprises an endogenous transmembrane region. In some embodiments, the humanized CD3E protein comprises a humanized extracellular region. In some embodiments, the humanized CD3E protein comprises a humanized signal peptide (e.g., amino acids 1-22 of SEQ ID NO: 2).

In some embodiments, the genetically-modified non-human animal described herein comprises a human or humanized CD3E gene. In some embodiments, the humanized CD3E gene comprises 9 exons. In some embodiments, the humanized CD3E gene comprises human or humanized exon 1, humanized exon 2, humanized exon 3, humanized exon 4, humanized exon 5, humanized exon 6, humanized exon 7, humanized exon 8, and/or humanized exon 9. In some embodiments, the humanized CD3E gene comprises human or humanized intron 1, humanized intron 2, humanized intron 3, humanized intron 4, humanized intron 5, humanized intron 6, humanized intron 7, and/or humanized intron 8. In some embodiments, the humanized CD3E gene comprises human or humanized 5′ UTR. In some embodiments, the humanized CD3E gene comprises human or humanized 3′ UTR. In some embodiments, the humanized CD3E gene comprises all or a portion of endogenous 5′ UTR (e.g., 5′ UTR of mouse CD3E gene). In some embodiments, the humanized CD3E gene comprises all or a portion of endogenous 3′ UTR (e.g., 3′ UTR of mouse CD3E gene).

In some embodiments, the genetically-modified non-human animal described herein comprises a portion of exon 2 (e.g., at least 20 bp nucleotides in exon 2), exons 3-8, and a portion of exon 9 (e.g., at least 40 bp nucleotides in exon 9) of human CD3E gene. In some embodiments, the genetically-modified non-human animal described herein comprises a portion of exon 2 (e.g., at least 20 bp nucleotides in exon 2), exons 3-6, and a portion of exon 7 (e.g., at least 20 bp nucleotides in exon 7) of human CD3E gene.

In some embodiments, the genetically-modified non-human animal (e.g., mouse) described herein comprises a replacement of a portion of exon 2 (e.g., at least 20 bp nucleotides in exon 2), exons 3-7, and a portion of exon 8 (e.g., at least 40 bp nucleotides in exon 8) of endogenous CD3E gene. In some embodiments, the replaced sequence is located at 44910829-44920694 of NCBI accession number NC_000075.7. In some embodiments, the genetically-modified non-human animal (e.g., mouse) described herein comprises a replacement of a portion of exon 2 (e.g., at least 20 bp nucleotides in exon 2), exons 3-5, and a portion of exon 6 (e.g., at least 10 bp nucleotides in exon 6) of endogenous CD3E gene. In some embodiments, the replaced sequence is located at 44912419-44920694 of NCBI accession number NC_000075.7.

In some embodiments, the present disclosure also provides a chimeric (e.g., humanized) CD3E nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD3E mRNA sequence (e.g., NM_007648.5), mouse CD3E amino acid sequence (e.g., NP_031674.1 (SEQ ID NO: 1)), or a portion thereof (e.g., exon 2, exon 3, exon 4, exon 5, exon 6, exon 7, and/or exon 8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD3E mRNA sequence (e.g., NM_000733.4), human CD3E amino acid sequence (e.g., NP_000724.1 (SEQ ID NO: 2)), or a portion thereof (e.g., exon 2, exon 3, exon 4, exon 5, exon 6, exon 7, exon 8, and/or exon 9).

In some embodiments, the sequence encoding amino acids 1-189 of mouse CD3E (SEQ ID NO: 1) is replaced. In some embodiments, the sequence is replaced by a sequence encoding a corresponding region of human CD3E (e.g., amino acids 1-207 of human CD3E (SEQ ID NO: 2)). In some embodiments, the sequence encoding amino acids 1-108 of mouse CD3E (SEQ ID NO: 1) is replaced. In some embodiments, the sequence is replaced by a sequence encoding a corresponding region of human CD3E (e.g., amino acids 1-126 of human CD3E (SEQ ID NO: 2)). In some embodiments, the sequence encoding the extracellular domain of mouse CD3E (e.g., SEQ ID NO: 1) is replaced. In some embodiments, the sequence is replaced by a sequence encoding a corresponding region (e.g., extracellular domain) of human CD3E (e.g., SEQ ID NO: 1).

In some embodiments, the nucleic acids as described herein are operably linked to a promotor or regulatory element, e.g., an endogenous mouse CD3E promotor, an inducible promoter, an enhancer, and/or mouse or human regulatory elements. In some embodiments, the nucleic acids described herein are operably linked to a human CD3E gene 5′ UTR and/or a human CD3E gene 3′ UTR. In some embodiments, the nucleic acids described herein are operably linked to an endogenous CD3E gene 5′ UTR and/or an endogenous CD3E gene 3′ UTR.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD3E nucleotide sequence (e.g., a portion of exon 2, exon 3, exon 4, exon 5, and a portion of exon 6 of NM_007648.5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD3E nucleotide sequence (e.g., exon 1, a portion of exon 2, a portion of exon 6, exon 7, and exon 8 of NM_007648.5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD3E nucleotide sequence (e.g., exon 1, a portion of exon 2, a portion of exon 7, exon 8, and exon 9 of NM_000733.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD3E nucleotide sequence (e.g., a portion of exon 2, exon 3, exon 4, exon 5, exon 6, and a portion of exon 7 of NM_000733.4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD3E amino acid sequence (e.g., an amino acid sequence encoded by a portion of exon 2, exon 3, exon 4, exon 5, and a portion of exon 6 of NM_007648.5; or amino acids 1-108 of NP_031674.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD3E amino acid sequence (e.g., an amino acid sequence encoded by exon 1, a portion of exon 2, a portion of exon 6, exon 7, and exon 8 of NM_007648.5; or amino acids 109-189 of NP_031674.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD3E amino acid sequence (e.g., an amino acid sequence encoded by exon 1, a portion of exon 2, a portion of exon 7, exon 8, and exon 9 of NM_000733.4; or amino acids 127-207 of NP_000724.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD3E amino acid sequence (e.g., an amino acid sequence encoded by a portion of exon 2, exon 3, exon 4, exon 5, exon 6, and a portion of exon 7 of NM_000733.4; or amino acids 1-126 of NP_000724.1 (SEQ ID NO: 2)).

### CD3D

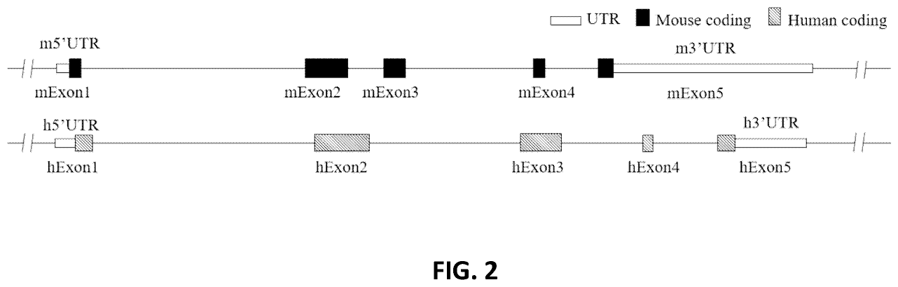

In human genomes, CD3D gene (Gene ID: 915) locus has five exons, exon 1, exon 2, exon 3, exon 4, and exon 5 (FIG. 2). The CD3D protein also has an extracellular region, a transmembrane region, and a cytoplasmic region, and the signal peptide is located at the extracellular region of CD3D. The nucleotide sequence for human CD3D mRNA is NM_000732.6, and the amino acid sequence for human CD3D is NP_000723.1 (SEQ ID NO: 4). The location for each exon and each region in human CD3D nucleotide sequence and amino acid sequence is listed below:

TABLE 3

| human CD3D | | NP_000723.1 (171 aa) |
|---|---|---|
| (approximate location) | NM_000732.6 | SEQ ID NO: 4 |
| Exon 1 | 1-153 | 1-18 |
| Exon 2 | 154-372 | 19-91 |
| Exon 3 | 373-504 | 92-135 |
| Exon 4 | 505-548 | 136-150 |
| Exon 5 | 549-701 | 151-171 |
| Signal peptide | 99-161 | 1-21 |
| Extracellular | 162-413 | 22-105 |
| Transmembrane | 414-476 | 106-126 |
| Cytoplasmic | 477-611 | 127-171 |
| Donor region in Examples | 1-701 | 1-171 |

The human CD3D gene (Gene ID: 915) is located in Chromosome 11 of the human genome, which is located from 118338954 to 118342705 of NC_000011.10 (GRCh38.p13 (GCF_000001405.39)). The 5′-UTR is from 118,342,705 to 118,342,608, exon 1 is from 118,342,705 to 118,342,553, the first intron is from 118,342,552 to 118,340,594, exon 2 is from 118,340,593 to 118,340,375, the second intron is from 118,340,374 to 118,339,907, exon 3 is from 118,339,906 to 118,339,775, the third intron is from 118,339,774 to 118,339,495, exon 4 is from 118,339,494 to 118,339,451, the fourth intron is from 118,339,450 to 118,339,228, exon 5 is from 118,339,227 to 118,339,075, the 3′-UTR is from 118,339,161 to 118,339,075, based on transcript NM_000732.6. All relevant information for human CD3D locus can be found in the NCBI website with Gene ID: 915, which is incorporated by reference herein in its entirety.

In mice, CD3D gene locus has five exons, exon 1, exon 2, exon 3, exon 4, and exon 5 (FIG. 2). The mouse CD3D protein also has an extracellular region, a transmembrane region, and a cytoplasmic region, and the signal peptide is located at the extracellular region of CD3D. The nucleotide sequence for mouse CD3D mRNA is NM_013487.3, the amino acid sequence for mouse CD3D is NP_038515.3 (SEQ ID NO: 3). The location for each exon and each region in the mouse CD3D nucleotide sequence and amino acid sequence is listed below:

TABLE 4

| Mouse CD3D | | NP_038515.3 (173 aa) |
|---|---|---|
| (approximate location) | NM_013487.3 | SEQ ID NO: 3 |
| Exon 1 | 1-155 | 1-18 |
| Exon 2 | 156-374 | 19-91 |
| Exon 3 | 375-506 | 92-135 |
| Exon 4 | 507-550 | 136-150 |
| Exon 5 | 551-1330 | 151-173 |
| Signal peptide | 101-163 | 1-21 |
| Extracellular | 164-415 | 22-105 |
| Transmembrane | 416-478 | 106-126 |
| Cytoplasmic | 479-619 | 127-173 |
| Replaced region in Examples | 1-1330 | 1-173 |

The mouse CD3D gene (Gene ID: 12500) is located in Chromosome 9 of the mouse genome, which is located from 44893067 to 44898350 of NC_000075.7 (GRCm39 (GCF_000001635.27)). The 5′-UTR is from 44,981,786 to 44,981,885, exon 1 is from 44,981,786 to 44,981,940, the first intron is from 44,981,941 to 44,984,969, exon 2 is from 44,984,970 to 44,985,188, the second intron is from 44,985,189 to 44,985,603, exon 3 is from 44,985,604 to 44,985,735, the third intron is from 44,985,736 to 44,986,056, exon 4 is from 44,986,057 to 44,986,100, the fourth intron is from 44,986,101 to 44,986,272, exon 5 is from 44,986,273 to 44,987,339, the 3′-UTR is from 44,986,345 to 44,987,339, based on transcript NM_013487.3. All relevant information for mouse Cd3d locus can be found in the NCBI website with Gene ID: 12500, which is incorporated by reference herein in its entirety.

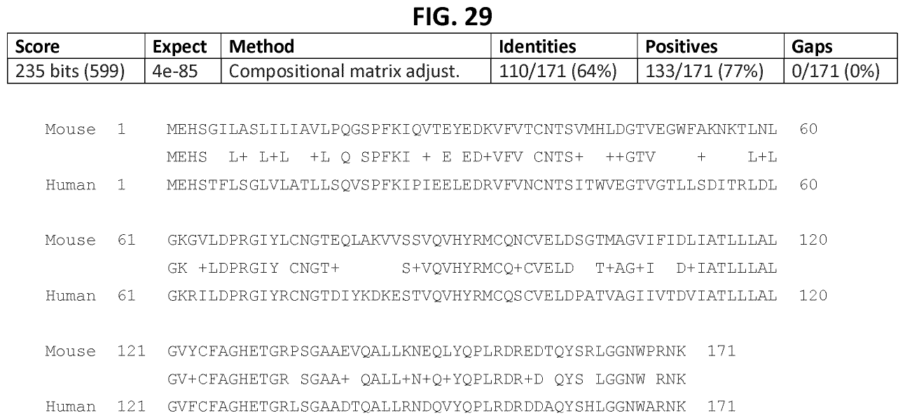

FIG. 29 shows the alignment between mouse CD3D amino acid sequence (NP_038515.3; SEQ ID NO: 3) and human CD3D amino acid sequence (NP_000723.1; SEQ ID NO: 4). Thus, the corresponding amino acid residue or region between human and mouse CD3D can be found in FIG. 29.

CD3D genes, proteins, and locus of the other species are also known in the art. For example, the gene ID for CD3D in Rattus norvegicus is 25710, the gene ID for CD3D in Macaca mulatta (Rhesus monkey) is 699582, the gene ID for CD3D in Canis lupus familiaris (dog) is 479419, and the gene ID for CD3D in Sus scrofa (pig) is 396661. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) CD3D nucleotide sequence and/or amino acid sequences. In some embodiments, the entire sequence of mouse exon 1, exon 2, exon 3, exon 4, exon 5, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, or 500 nucleotides, or at least 1, 2, 3, 4, 5, 6, 7, 8, 9, 10, 20, 30, 40, 50, 60, 70, 80, 90, 100, 110, 120, 130, 140, 150, 160, or 170 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, signal peptide, extracellular region, transmembrane region, or cytoplasmic region. In some embodiments, a region, a portion, or the entire sequence of mouse exon 1, exon 2, exon 3, exon 4, and/or exon 5 are replaced by the human exon 1, exon 2, exon 3, exon 4, and/or exon 5 sequence.

In some embodiments, the genetically-modified non-human animal described herein comprises a sequence encoding a human or humanized CD3D protein. In some embodiments, the humanized CD3D protein comprises an endogenous cytoplasmic region. In some embodiments, the humanized CD3D protein comprises an endogenous transmembrane region. In some embodiments, the humanized CD3D protein comprises a humanized extracellular region. In some embodiments, the humanized CD3D protein comprises a humanized signal peptide (e.g., amino acids 1-21 of SEQ ID NO: 4).

In some embodiments, the genetically-modified non-human animal described herein comprises a human or humanized CD3D gene. In some embodiments, the humanized CD3D gene comprises 5 exons. In some embodiments, the humanized CD3D gene comprises humanized exon 1, humanized exon 2, humanized exon 3, humanized exon 4, and/or humanized exon 5. In some embodiments, the humanized CD3D gene comprises humanized intron 1, humanized intron 2, humanized intron 3, and/or humanized intron 4. In some embodiments, the humanized CD3D gene comprises human or humanized 5′ UTR. In some embodiments, the humanized CD3D gene comprises human or humanized 3′ UTR.

In some embodiments, the present disclosure also provides a chimeric (e.g., humanized) CD3D nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD3D mRNA sequence (e.g., NM_013487.3), mouse CD3D amino acid sequence (e.g., NP_038515.3 (SEQ ID NO: 3)), or a portion thereof (e.g., exon 1, exon 2, exon 3, exon 4, and/or exon 5); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD3D mRNA sequence (e.g., NM_000732.6), human CD3D amino acid sequence (e.g., NP_000723.1 (SEQ ID NO: 4)), or a portion thereof (e.g., exon 1, exon 2, exon 3, exon 4, and/or exon 5).

In some embodiments, the sequence encoding amino acids 1-173 of mouse CD3D (SEQ ID NO: 3) is replaced. In some embodiments, the sequence is replaced by a sequence encoding a corresponding region of human CD3D (e.g., amino acids 1-171 of human CD3D (SEQ ID NO: 4)). In some embodiments, the sequence encoding amino acids 1-105 of mouse CD3D (SEQ ID NO: 3) is replaced. In some embodiments, the sequence is replaced by a sequence encoding a corresponding region of human CD3D (e.g., amino acids 1-105 of human CD3D (SEQ ID NO: 4)). In some embodiments, the sequence encoding the extracellular domain of mouse CD3D (e.g., SEQ ID NO: 3) is replaced. In some embodiments, the sequence is replaced by a sequence encoding a corresponding region (e.g., extracellular domain) of human CD3D (e.g., SEQ ID NO: 4).

In some embodiments, the nucleic acids as described herein are operably linked to a promotor or regulatory element, e.g., an endogenous mouse CD3D promotor, an inducible promoter, an enhancer, and/or mouse or human regulatory elements. In some embodiments, the nucleic acids described herein are operably linked to a human CD3D gene 5′ UTR and/or a human CD3D gene 3′ UTR.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD3D nucleotide sequence (e.g., exon 1, exon 2, exon 3, exon 4, and/or exon 5 of NM_013487.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD3D nucleotide sequence (e.g., exon 1, exon 2, exon 3, exon 4, and/or exon 5 of NM_013487.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD3D nucleotide sequence (e.g., exon 1, exon 2, exon 3, exon 4, and/or exon 5 of NM_000732.6).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD3D nucleotide sequence (e.g., exon 1, exon 2, exon 3, exon 4, and/or exon 5 of NM_000732.6).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD3D amino acid sequence (e.g., an amino acid sequence encoded by exon 1, exon 2, exon 3, exon 4, and/or exon 5 of NM_013487.3; or amino acids 1-173 of NP_038515.3 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD3D amino acid sequence (e.g., an amino acid sequence encoded by exon 1, exon 2, exon 3, exon 4, and/or exon 5 of NM_013487.3; or amino acids 1-173 of NP_038515.3 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD3D amino acid sequence (e.g., an amino acid sequence encoded by exon 1, exon 2, exon 3, exon 4, and/or exon 5 of NM_000732.6; or amino acids 1-171 of NP_000723.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD3D amino acid sequence (e.g., an amino acid sequence encoded by exon 1, exon 2, exon 3, exon 4, and/or exon 5 of NM_000732.6; or amino acids 1-171 of NP_000723.1 (SEQ ID NO: 4)).

### CD3G

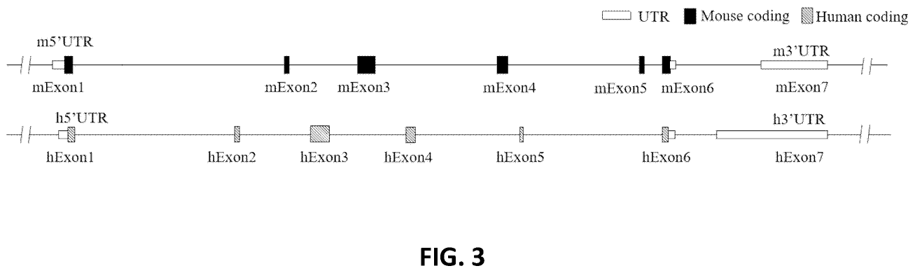

In human genomes, CD3G gene (Gene ID: 917) locus has seven exons, exon 1, exon 2, exon 3, exon 4, exon 5, exon 6, and exon 7 (FIG. 3). The CD3G protein also has an extracellular region, a transmembrane region, and a cytoplasmic region, and the signal peptide is located at the extracellular region of CD3G. The nucleotide sequence for human CD3G mRNA is NM_000073.3, and the amino acid sequence for human CD3G is NP_000064.1 (SEQ ID NO: 6). The location for each exon and each region in human CD3G nucleotide sequence and amino acid sequence is listed below:

TABLE 5

| human CD3G | | NP_000064.1 (182 aa) |
|---|---|---|
| (approximate location) | NM_000073.3 | SEQ ID NO: 6 |
| Exon 1 | 1-135 | 1-18 |
| Exon 2 | 136-159 | 19-26 |
| Exon 3 | 160-387 | 27-102 |
| Exon 4 | 388-519 | 103-146 |
| Exon 5 | 520-563 | 147-161 |
| Exon 6 | 564-647 | 162-182 |
| Exon 7 | 648-2690 | Non coding |
| Signal peptide | 81-146 | 1-22 |
| Extracellular | 147-428 | 23-116 |
| Transmembrane | 429-491 | 117-137 |
| Cytoplasmic | 492-626 | 138-182 |
| Donor region in Examples | 1-2690 | 1-182 |

The human CD3G gene (Gene ID: 917) is located in Chromosome 11 of the human genome, which is located from 118344344 to 118355161 of NC_000011.10 (GRCh38.p13 (GCF_000001405.39)). The 5′-UTR is from 118,344,344 to 118,344,423, exon 1 is from 118,344,344 to 118,344,478, the first intron is from 118,344,479 to 118,349,026, exon 2 is from 118,349,027 to 118,349,050, the second intron is from 118,349,051 to 118,349,742, exon 3 is from 118,349,743 to 118,349,970, the third intron is from 118,349,971 to 118,350,551, exon 4 is from 118,350,552 to 118,350,683, the fourth intron is from 118,350,684 to 118,351,627, exon 5 is from 118,351,628 to 118,351,671, the fifth intron is from 118,351,672 to 118,352,403, exon 6 is from 118,352,404 to 118,352,487, the sixth intron is from 118,352,488 to 118,353,118, exon 7 is from 118,353,119 to 118,355,161, the 3′-UTR is from 118,352,470 to 118,352,487 and 118,353,119 to 118,355,161, based on transcript NM_000073.3. All relevant information for human CD3G locus can be found in the NCBI website with Gene ID: 917, which is incorporated by reference herein in its entirety.

In mice, CD3G gene locus has seven exons, exon 1, exon 2, exon 3, exon 4, exon 5, exon 6, and exon 7 (FIG. 3). The mouse CD3G protein also has an extracellular region, a transmembrane region, and a cytoplasmic region, and the signal peptide is located at the extracellular region of CD3G. The nucleotide sequence for mouse CD3G mRNA is NM_009850.2, the amino acid sequence for mouse CD3G is NP_033980.1 (SEQ ID NO: 5). The location for each exon and each region in the mouse CD3G nucleotide sequence and amino acid sequence is listed below:

TABLE 6

| Mouse CD3G | | NP_033980.1 (182 aa) |
|---|---|---|
| (approximate location) | NM_009850.2 | SEQ ID NO: 5 |
| Exon 1 | 1-166 | 1-18 |
| Exon 2 | 167-190 | 19-26 |
| Exon 3 | 191-418 | 27-102 |
| Exon 4 | 419-550 | 103-146 |
| Exon 5 | 551-594 | 147-161 |
| Exon 6 | 595-677 | 162-182 |
| Exon 7 | 678-1023 | Non coding |
| Signal peptide | 112-177 | 1-22 |
| Extracellular | 178-459 | 23-116 |
| Transmembrane | 460-522 | 117-137 |
| Cytoplasmic | 523-657 | 138-182 |
| Replaced region in Examples | 1-1023 | 1-182 |

The mouse CD3G gene (Gene ID: 12502) is located in Chromosome 9 of the mouse genome, which is located from 44880870 to 44891729 of NC_000075.7 (GRCm39 (GCF_000001635.27)). The 5′-UTR is from 44,980,431 to 44,980,321, exon 1 is from 44,980,431 to 44,980,266, the first intron is from 44,980,265 to 44,975,271, exon 2 is from 44,975,270 to 44,975,247, the second intron is from 44,975,246 to 44,974,391, exon 3 is from 44,974,390 to 44,974,163, the third intron is from 44,974,162 to 44,973,638, exon 4 is from 44,973,637 to 44,973,506, the fourth intron is from 44,973,505 to 44,971,307, exon 5 is from 44,971,306 to 44,971,263, the fifth intron is from 44,971,262 to 44,970,774, exon 6 is from 44,970,773 to 44,970,691, the sixth intron is from 44,970,690 to 44,969,918, exon 7 is from 44,969,917 to 44,969,572, the 3′-UTR is from 44,970,707 to U.S. Pat. Nos. 44,970,691 and 44,969,917 to 44,969,572, based on transcript NM_009850.2. All relevant information for mouse CD3G locus can be found in the NCBI website with Gene ID: 12502, which is incorporated by reference herein in its entirety.

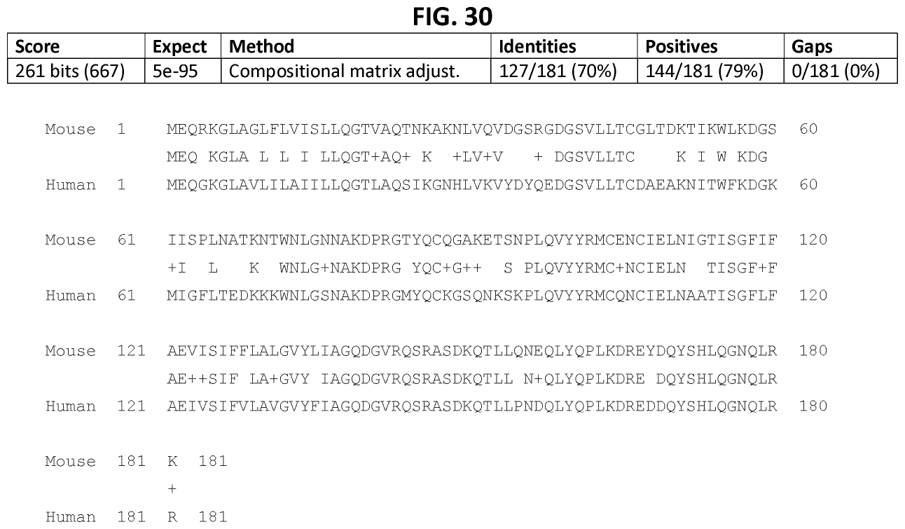

FIG. 30 shows the alignment between mouse CD3G amino acid sequence (NP_033980.1; SEQ ID NO: 5) and human CD3G amino acid sequence (NP_000064.1; SEQ ID NO: 6). Thus, the corresponding amino acid residue or region between human and mouse CD3G can be found in FIG. 30.

CD3G genes, proteins, and locus of the other species are also known in the art. For example, the gene ID for CD3G in Rattus norvegicus is 300678, the gene ID for CD3G in Macaca mulatta (Rhesus monkey) is 705270, the gene ID for CD3G in Canis lupus familiaris (dog) is 489383, and the gene ID for CD3G in Sus scrofa (pig) is 494013. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) CD3G nucleotide sequence and/or amino acid sequences. In some embodiments, the entire sequence of mouse exon 1, exon 2, exon 3, exon 4, exon 5, exon 6, exon 7, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, or 500 nucleotides, or at least 1, 2, 3, 4, 5, 6, 7, 8, 9, 10, 20, 30, 40, 50, 60, 70, 80, 90, 100, 110, 120, 130, 140, 150, 160, or 170 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, signal peptide, extracellular region, transmembrane region, or cytoplasmic region. In some embodiments, a region, a portion, or the entire sequence of mouse exon 1, exon 2, exon 3, exon 4, exon 5, exon 6, and/or exon 7 are replaced by the human exon 1, exon 2, exon 3, exon 4, exon 5, exon 6, and/or exon 7 sequence.

In some embodiments, the genetically-modified non-human animal described herein comprises a sequence encoding a human or humanized CD3G protein. In some embodiments, the humanized CD3G protein comprises an endogenous cytoplasmic region. In some embodiments, the humanized CD3G protein comprises an endogenous transmembrane region. In some embodiments, the humanized CD3G protein comprises a humanized extracellular region. In some embodiments, the humanized CD3G protein comprises a humanized signal peptide (e.g., amino acids 1-22 of SEQ ID NO: 6).

In some embodiments, the genetically-modified non-human animal described herein comprises a human or humanized CD3G gene. In some embodiments, the humanized CD3G gene comprises 7 exons. In some embodiments, the humanized CD3G gene comprises humanized exon 1, humanized exon 2, humanized exon 3, humanized exon 4, humanized exon 5, humanized exon 6, and/or humanized exon 7. In some embodiments, the humanized CD3G gene comprises humanized intron 1, humanized intron 2, humanized intron 3, humanized intron 4, humanized intron 5, and/or humanized intron 6. In some embodiments, the humanized CD3G gene comprises human or humanized 5′ UTR. In some embodiments, the humanized CD3G gene comprises human or humanized 3′ UTR.

In some embodiments, the present disclosure also provides a chimeric (e.g., humanized) CD3G nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD3G mRNA sequence (e.g., NM_009850.2), mouse CD3G amino acid sequence (e.g., NP_033980.1 (SEQ ID NO: 5)), or a portion thereof (e.g., exon 1, exon 2, exon 3, exon 4, exon 5, exon 6, and/or exon 7); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD3G mRNA sequence (e.g., NM_000073.3), human CD3G amino acid sequence (e.g., NP_000064.1 (SEQ ID NO: 6)), or a portion thereof (e.g., exon 1, exon 2, exon 3, exon 4, exon 5, exon 6, and/or exon 7).

In some embodiments, the sequence encoding amino acids 1-182 of mouse CD3G (SEQ ID NO: 5) is replaced. In some embodiments, the sequence is replaced by a sequence encoding a corresponding region of human CD3G (e.g., amino acids 1-182 of human CD3G (SEQ ID NO: 6)). In some embodiments, the sequence encoding amino acids 1-116 of mouse CD3G (SEQ ID NO: 5) is replaced. In some embodiments, the sequence is replaced by a sequence encoding a corresponding region of human CD3G (e.g., amino acids 1-116 of human CD3G (SEQ ID NO: 6)). In some embodiments, the sequence encoding the extracellular domain of mouse CD3G (e.g., SEQ ID NO: 5) is replaced. In some embodiments, the sequence is replaced by a sequence encoding a corresponding region (e.g., extracellular domain) of human CD3G (e.g., SEQ ID NO: 6).

In some embodiments, the nucleic acids as described herein are operably linked to a promotor or regulatory element, e.g., an endogenous mouse CD3G promotor, an inducible promoter, an enhancer, and/or mouse or human regulatory elements. In some embodiments, the nucleic acids described herein are operably linked to a human CD3G gene 5′ UTR and/or a human CD3G gene 3′ UTR.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD3G nucleotide sequence (e.g., exon 1, exon 2, exon 3, exon 4, exon 5, exon 6, and/or exon 7 of NM_009850.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD3G nucleotide sequence (e.g., exon 1, exon 2, exon 3, exon 4, exon 5, exon 6, and/or exon 7 of NM_009850.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD3G nucleotide sequence (e.g., exon 1, exon 2, exon 3, exon 4, exon 5, exon 6, and/or exon 7 of NM_000073.3).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD3G nucleotide sequence (e.g., exon 1, exon 2, exon 3, exon 4, exon 5, exon 6, and/or exon 7 of NM_000073.3).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD3G amino acid sequence (e.g., an amino acid sequence encoded by exon 1, exon 2, exon 3, exon 4, exon 5, exon 6, and/or exon 7 of NM_009850.2; or amino acids 1-182 of NP_033980.1 (SEQ ID NO: 5)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD3G amino acid sequence (e.g., an amino acid sequence encoded by exon 1, exon 2, exon 3, exon 4, exon 5, exon 6, and/or exon 7 of NM_009850.2; or amino acids 1-182 of NP_033980.1 (SEQ ID NO: 5)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD3G amino acid sequence (e.g., an amino acid sequence encoded by exon 1, exon 2, exon 3, exon 4, exon 5, exon 6, and/or exon 7 of NM_000073.3; or amino acids 1-182 of NP_000064.1 (SEQ ID NO: 6)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD3G amino acid sequence (e.g., an amino acid sequence encoded by exon 1, exon 2, exon 3, exon 4, exon 5, exon 6, and/or exon 7 of NM_000073.3; or amino acids 1-182 of NP_000064.1 (SEQ ID NO: 6)).

In some embodiments, the genetically-modified non-human animal (e.g., mouse) described herein comprises a replacement of exon 1-exon 5 of endogenous CD3G gene and exon 1-exon 7 of endogenous CD3G gene. In some embodiments, the replaced sequence is located at 44880866-44898910 of NCBI accession number NC_000075.7.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CD3E locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wildtype amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized CD3E gene or a humanized CD3E nucleic acid. In some embodiments, at least one or more portions of the gene or the nucleic acid is from the human CD3E gene, at least one or more portions of the gene or the nucleic acid is from a non-human CD3E gene. In some embodiments, the gene or the nucleic acid comprises a sequence that encodes a CD3E protein. The encoded CD3E protein is functional or has at least one activity of the human CD3E protein or the non-human CD3E protein, e.g., associate with human or endogenous CD3 gamma (γ), delta (δ) and/or zeta (ζ) polypeptide, form a T cell co-receptor, associate with human or endogenous T cell receptor, activating T cell (e.g., inducing T cell division), increasing CD3 expression, increasing expression of CD69 and/or CD25, increasing production of proinflammatory cytokines, inducing activation and proliferation of immune cells, increasing the production of cytokines (e.g., TGF-β and IFN-γ), and/or upregulating the immune response.

In some embodiments, the chimeric gene or the chimeric nucleic acid is a humanized (or human) CD3D gene or a humanized (or human) CD3D nucleic acid. In some embodiments, at least one or more portions of the gene or the nucleic acid is from the human CD3D gene, at least one or more portions of the gene or the nucleic acid is from a non-human CD3D gene. In some embodiments, the gene or the nucleic acid comprises a sequence that encodes a CD3D protein. The encoded CD3D protein is functional or has at least one activity of the human CD3D protein or the non-human CD3D protein, e.g., associate with human or endogenous CD3 gamma (γ), epsilon (c), and/or zeta (ζ) polypeptide, form a T cell co-receptor, associate with human or endogenous T cell receptor, activating T cell (e.g., inducing T cell division), increasing CD3 expression, increasing expression of CD69 and/or CD25, increasing production of proinflammatory cytokines, inducing activation and proliferation of immune cells, increasing the production of cytokines (e.g., TGF-β and IFN-γ), and/or upregulating the immune response.

In some embodiments, the chimeric gene or the chimeric nucleic acid is a humanized (or human) CD3G gene or a humanized (or human) CD3G nucleic acid. In some embodiments, at least one or more portions of the gene or the nucleic acid is from the human CD3G gene, at least one or more portions of the gene or the nucleic acid is from a non-human CD3G gene. In some embodiments, the gene or the nucleic acid comprises a sequence that encodes a CD3G protein. The encoded CD3 G protein is functional or has at least one activity of the human CD3 G protein or the non-human CD3G protein, e.g., associate with human or endogenous CD3 delta (δ), epsilon (c), and/or zeta (ζ) polypeptide, form a T cell co-receptor, associate with human or endogenous T cell receptor, activating T cell (e.g., inducing T cell division), increasing CD3 expression, increasing expression of CD69 and/or CD25, increasing production of proinflammatory cytokines, inducing activation and proliferation of immune cells, increasing the production of cytokines (e.g., TGF-β and IFN-γ), and/or upregulating the immune response.

In some embodiments, the chimeric protein or the chimeric polypeptide is a humanized (or human) CD3E protein or a humanized (or human) CD3E polypeptide. In some embodiments, at least one or more portions of the amino acid sequence of the protein or the polypeptide is from a human CD3E protein, and at least one or more portions of the amino acid sequence of the protein or the polypeptide is from a non-human CD3E protein. The humanized CD3E protein or the humanized CD3E polypeptide is functional or has at least one activity of the human CD3E protein or the non-human CD3E protein. In some embodiments, the chimeric protein or the chimeric polypeptide is a humanized CD3D protein or a humanized CD3D polypeptide. In some embodiments, at least one or more portions of the amino acid sequence of the protein or the polypeptide is from a human CD3D protein, and at least one or more portions of the amino acid sequence of the protein or the polypeptide is from a non-human CD3D protein. The humanized CD3D protein or the humanized CD3D polypeptide is functional or has at least one activity of the human CD3D protein or the non-human CD3D protein. In some embodiments, the chimeric protein or the chimeric polypeptide is a humanized CD3G protein or a humanized CD3G polypeptide. In some embodiments, at least one or more portions of the amino acid sequence of the protein or the polypeptide is from a human CD3G protein, and at least one or more portions of the amino acid sequence of the protein or the polypeptide is from a non-human CD3G protein. The humanized CD3G protein or the humanized CD3G polypeptide is functional or has at least one activity of the human CD3G protein or the non-human CD3G protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Mitroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/01a. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sy; or 50% C57BL/6-50% 129). In some embodiments, the non-human animal is a rodent. In some embodiments, the non-human animal is a mouse having a BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr [symbol] C57BL/Ola [symbol] C57BL, C58, CBA/Br, CBA/Ca, CBA/J, CBA/st, or CBA/H background.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized CD3E, CD3D, and/or CD3G animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γcnull mice (Ito, M. et al., NOD/SCID/γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human CD3E locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc null mice, nude mice, Rag1 and/or Rag2 knockout mice, NOD-Prkdcscid IL-2rγnul mice, NOD-Rag 1−/−-IL2rg−/− (NRG) mice, Rag 2−/−-IL2rg−/− (RG) mice, and a combination thereof. These genetically modified animals are described, e.g., in US20150106961, which is incorporated herein by reference in its entirety. In some embodiments, the mouse can include a replacement of all or part of mature CD3E coding sequence with human mature CD3E coding sequence.

Also provided herein are genetically modified non-human animals that comprise a modification of an endogenous non-human CD3E locus. In some embodiments, the modification can comprise a human nucleic acid sequence encoding at least a portion of a mature CD3E protein (e.g., at least 10%, 20%, 30%, 40%, 50%, 60%, 70%, 80%, 90%, 95%, 96%, 97%, 98%, or 99% identical to the mature CD3E protein sequence). Also provided herein are genetically modified non-human animals that comprise a modification of an endogenous non-human CD3D locus. In some embodiments, the modification can comprise a human nucleic acid sequence encoding at least a portion of a mature CD3D protein (e.g., at least 10%, 20%, 30%, 40%, 50%, 60%, 70%, 80%, 90%, 95%, 96%, 97%, 98%, or 99% identical to the mature CD3D protein sequence). Also provided herein are genetically modified non-human animals that comprise a modification of an endogenous non-human CD3G locus. In some embodiments, the modification can comprise a human nucleic acid sequence encoding at least a portion of a mature CD3G protein (e.g., at least 10%, 20%, 30%, 40%, 50%, 60%, 70%, 80%, 90%, 95%, 96%, 97%, 98%, or 99% identical to the mature CD3G protein sequence). Although genetically modified cells are also provided that can comprise the modifications described herein (e.g., ES cells, somatic cells), in many embodiments, the genetically modified non-human animals comprise the modification of the endogenous CD3E, CD3D, and/or CD3G locus in the germline of the animal.

Genetically modified animals can express a human CD3E and/or a chimeric (e.g., humanized) CD3E from endogenous mouse loci, wherein the endogenous mouse CD3E gene has been replaced with a human CD3E gene and/or a nucleotide sequence that encodes a region of human CD3E sequence or an amino acid sequence that is at least 10%, 20%, 30%, 40%, 50%, 60%, 70%, 80%, 90%, 95%, 96%, 97%, 98%, or 99% identical to the human CD3E sequence. In various embodiments, an endogenous non-human CD3E locus is modified in whole or in part to comprise human nucleic acid sequence encoding at least one protein-coding sequence of a mature CD3E protein. Genetically modified animals can express a human CD3D and/or a chimeric (e.g., humanized) CD3D from endogenous mouse loci, wherein the endogenous mouse CD3D gene has been replaced with a human CD3D gene and/or a nucleotide sequence that encodes a region of human CD3D sequence or an amino acid sequence that is at least 10%, 20%, 30%, 40%, 50%, 60%, 70%, 80%, 90%, 95%, 96%, 97%, 98%, or 99% identical to the human CD3D sequence. In various embodiments, an endogenous non-human CD3D locus is modified in whole or in part to comprise human nucleic acid sequence encoding at least one protein-coding sequence of a mature CD3D protein. Genetically modified animals can express a human CD3G and/or a chimeric (e.g., humanized) CD3G from endogenous mouse loci, wherein the endogenous mouse CD3G gene has been replaced with a human CD3G gene and/or a nucleotide sequence that encodes a region of human CD3G sequence or an amino acid sequence that is at least 10%, 20%, 30%, 40%, 50%, 60%, 70%, 80%, 90%, 95%, 96%, 97%, 98%, or 99% identical to the human CD3G sequence. In various embodiments, an endogenous non-human CD3G locus is modified in whole or in part to comprise human nucleic acid sequence encoding at least one protein-coding sequence of a mature CD3G protein.

In some embodiments, the genetically modified mice express the human CD3E and/or chimeric CD3E (e.g., humanized CD3E) from endogenous loci that are under control of mouse promoters and/or mouse regulatory elements. The replacement(s) at the endogenous mouse loci provide non-human animals that express human CD3E or chimeric CD3E (e.g., humanized CD3E) in appropriate cell types and in a manner that does not result in the potential pathologies observed in some other transgenic mice known in the art. The human CD3E or the chimeric CD3E (e.g., humanized CD3E) expressed in animal can maintain one or more functions of the wildtype mouse or human CD3E in the animal. For example, human or non-human CD3E can form T cell co-receptor, and then interact with T cell receptor. Upon binding with antigens presented by major histocompatibility complex I (MHC I) or MHCII, the T cell receptor with its co-receptor can upregulate immune response, e.g., upregulate immune response by at least 10%, 20%, 30%, 40%, or 50%. Furthermore, in some embodiments, the animal does not express endogenous CD3E. As used herein, the term “endogenous CD3E” refers to CD3E protein that is expressed from an endogenous CD3E nucleotide sequence of the non-human animal (e.g., mouse) before any genetic modification.

In some embodiments, the genetically modified mice express the human CD3D and/or chimeric CD3D (e.g., humanized CD3D) from endogenous loci that are under control of mouse promoters and/or mouse regulatory elements. In some embodiments, the genetically modified mice express the human CD3D and/or chimeric CD3D (e.g., humanized CD3D) from endogenous loci that are under control of human promoters and/or human regulatory elements. In some embodiments, the genetically modified mice express the human CD3G and/or chimeric CD3G (e.g., humanized CD3G) from endogenous loci that are under control of mouse promoters and/or mouse regulatory elements. In some embodiments, the genetically modified mice express the human CD3G and/or chimeric CD3G (e.g., humanized CD3G) from endogenous loci that are under control of human promoters and/or human regulatory elements.

The genome of the animal can comprise a sequence encoding an amino acid sequence that is at least 50%, 60%, 70%, 75%, 80%, 85%, 90%, 95%, 99%, or 100% identical to human CD3E (NP_000724.1) (SEQ ID NO: 2). In some embodiments, the genome comprises a sequence encoding an amino acid sequence that is at least 50%, 60%, 70%, 75%, 80%, 85%, 90%, 95%, 99%, or 100% identical to SEQ ID NO: 2 or 66. The genome of the animal can comprise a sequence encoding an amino acid sequence that is at least 50%, 60%, 70%, 75%, 80%, 85%, 90%, 95%, 99%, or 100% identical to human CD3D (NP_000723.1) (SEQ ID NO: 4). In some embodiments, the genome comprises a sequence encoding an amino acid sequence that is at least 50%, 60%, 70%, 75%, 80%, 85%, 90%, 95%, 99%, or 100% identical to SEQ ID NO: 4. The genome of the animal can comprise a sequence encoding an amino acid sequence that is at least 50%, 60%, 70%, 75%, 80%, 85%, 90%, 95%, 99%, or 100% identical to human CD3G (NP_000064.1) (SEQ ID NO: 6). In some embodiments, the genome comprises a sequence encoding an amino acid sequence that is at least 50%, 60%, 70%, 75%, 80%, 85%, 90%, 95%, 99%, or 100% identical to SEQ ID NO: 6.

The genome of the genetically modified animal can comprise a replacement at an endogenous CD3E gene locus of a sequence encoding a region of endogenous CD3E with a sequence encoding a corresponding region of human CD3E. In some embodiments, the sequence that is replaced is any sequence within the endogenous CD3E gene locus, e.g., exon 1, exon 2, exon 3, exon 4, exon 5, exon 6, exon 7, exon 8, 5′-UTR, 3′-UTR, the first intron, the second intron, the third intron, the fourth intron, the fifth intron, the sixth intron, the seventh intron, etc. In some embodiments, the sequence that is replaced is within the regulatory region of the endogenous CD3E gene. In some embodiments, the sequence that is replaced is exon 2, exon 3, exon 4, exon 5, exon 6 or part thereof, of an endogenous mouse CD3E gene locus. The genome of the genetically modified animal can comprise a replacement at an endogenous CD3D gene locus of a sequence encoding a region of endogenous CD3D with a sequence encoding a corresponding region of human CD3D. In some embodiments, the sequence that is replaced is any sequence within the endogenous CD3D gene locus, e.g., exon 1, exon 2, exon 3, exon 4, exon 5, 5′-UTR, 3′-UTR, the first intron, the second intron, the third intron, the fourth intron, etc. In some embodiments, the sequence that is replaced is within the regulatory region of the endogenous CD3D gene. In some embodiments, the sequence that is replaced is exon 1, exon 2, exon 3, exon 4, exon 5 or part thereof, of an endogenous mouse CD3D gene locus. The genome of the genetically modified animal can comprise a replacement at an endogenous CD3G gene locus of a sequence encoding a region of endogenous CD3G with a sequence encoding a corresponding region of human CD3G. In some embodiments, the sequence that is replaced is any sequence within the endogenous CD3G gene locus, e.g., exon 1, exon 2, exon 3, exon 4, exon 5, exon 6, exon 7, 5′-UTR, 3′-UTR, the first intron, the second intron, the third intron, the fourth intron, the fifth intron, the sixth intron, etc. In some embodiments, the sequence that is replaced is within the regulatory region of the endogenous CD3G gene. In some embodiments, the sequence that is replaced is exon 1, exon 2, exon 3, exon 4, exon 5, exon 6, exon 7 or part thereof, of an endogenous mouse CD3G gene locus.

The genetically modified animal can have one or more cells expressing a human or chimeric CD3E (e.g., humanized CD3E) having an extracellular region and a cytoplasmic region, wherein the extracellular region comprises a sequence that is at least 50%, 60%, 70%, 80%, 90%, 95%, 99% identical to the extracellular region of human CD3E. In some embodiments, the extracellular region of the humanized CD3E has a sequence that has at least 10, 20, 30, 40, 50, 60, 70, 80, 90, 100, 110, or 120 amino acids (e.g., contiguously or non-contiguously) that are identical to human CD3E. Because human CD3E and non-human CD3E (e.g., mouse CD3E) sequences, in many cases, are different, antibodies that bind to human CD3E will not necessarily have the same binding affinity with non-human CD3E or have the same effects to non-human CD3E. Therefore, the genetically modified animal having a human or a humanized extracellular region can be used to better evaluate the effects of anti-human CD3E antibodies in an animal model. In some embodiments, the genome of the genetically modified animal comprises a sequence encoding an amino acid sequence that corresponds to part or the entire sequence of exon 2, exon 3, exon 4, exon 5, exon 6, and/or exon 7 of human CD3E, part or the entire sequence of extracellular region of human CD3E (with or without signal peptide), or part or the entire sequence of amino acids 1-126 of SEQ ID NO: 2.

The genetically modified animal can have one or more cells expressing a human or chimeric CD3D (e.g., humanized CD3D). In some embodiments, the genome of the genetically modified animal comprises a sequence encoding an amino acid sequence that corresponds to part or the entire sequence of exon 1, exon 2, exon 3, exon 4, and/or exon 5 of human CD3D, part or the entire sequence of extracellular region of human CD3D (with or without signal peptide), or part or the entire sequence of SEQ ID NO: 4. The genetically modified animal can have one or more cells expressing a human or chimeric CD3G (e.g., humanized CD3G). In some embodiments, the genome of the genetically modified animal comprises a sequence encoding an amino acid sequence that corresponds to part or the entire sequence of exon 1, exon 2, exon 3, exon 4, exon 5, exon 6, and/or exon 7 of human CD3G, part or the entire sequence of extracellular region of human CD3G (with or without signal peptide), or part or the entire sequence of SEQ ID NO: 6.

In some embodiments, the non-human animal can have, at an endogenous CD3E gene locus, a nucleotide sequence encoding a human CD3E or a chimeric human/non-human CD3E polypeptide, wherein a human portion of the chimeric human/non-human CD3E polypeptide comprises a portion of human CD3E extracellular domain, and wherein the animal expresses a functional CD3E on a surface of a cell (e.g., T cell) of the animal. The human portion of the chimeric human/non-human CD3E polypeptide can comprise an amino acid encoded by a portion of exon 2, exon 3, exon 4, exon 5, exon 6, and/or a portion of exon 7 of human CD3E gene. In some embodiments, the human portion of the chimeric human/non-human CD3E polypeptide can comprise a sequence that is at least 80%, 85%, 90%, 95%, or 99% identical to amino acids 1-126 of SEQ ID NO: 2. In some embodiments, the non-human animal can have, at an endogenous CD3D gene locus, a nucleotide sequence encoding a human CD3D or a chimeric human/non-human CD3D polypeptide, and wherein the animal expresses a functional CD3D on a surface of a cell (e.g., T cell) of the animal. The human portion of the chimeric human/non-human CD3D polypeptide can comprise an amino acid encoded by exon 1, exon 2, exon 3, exon 4, and/or exon 5 of human CD3D gene. In some embodiments, the human portion of the chimeric human/non-human CD3D polypeptide can comprise a sequence that is at least 80%, 85%, 90%, 95%, or 99% identical to SEQ ID NO: 4. In some embodiments, the non-human animal can have, at an endogenous CD3G gene locus, a nucleotide sequence encoding a human CD3G or a chimeric human/non-human CD3G polypeptide, and wherein the animal expresses a functional CD3G on a surface of a cell (e.g., T cell) of the animal. The human portion of the chimeric human/non-human CD3G polypeptide can comprise an amino acid encoded by exon 1, exon 2, exon 3, exon 4, exon 5, exon 6, and/or exon 7 of human CD3DG gene. In some embodiments, the human portion of the chimeric human/non-human CD3G polypeptide can comprise a sequence that is at least 80%, 85%, 90%, 95%, or 99% identical to SEQ ID NO: 6.

In some embodiments, the non-human portion of the chimeric human/non-human CD3E polypeptide comprises transmembrane and/or cytoplasmic regions of an endogenous non-human CD3E polypeptide. There may be several advantages that are associated with the transmembrane and/or cytoplasmic regions of an endogenous non-human CD3E polypeptide. For example, once CD3E forms a CD3 co-receptor complex, it can properly transmit extracellular signals into the cells and initiate the downstream pathway. A human or humanized transmembrane and/or cytoplasmic regions may not function properly in non-human animal cells. In some embodiments, a few extracellular amino acids that are close to the transmembrane region of CD3E are also derived from endogenous sequence. These amino acids can also be important for transmembrane signal transmission. In some embodiments, the chimeric human/non-human CD3E is functional. In some embodiments, the non-human animal with chimeric human/non-human CD3E is healthy (e.g., without any obvious change in body weight or size of organs, such as thymus).

Furthermore, the genetically modified animal can be heterozygous with respect to the replacement at the endogenous CD3E, CD3D, and/or CD3G locus, or homozygous with respect to the replacement at the endogenous CD3E, CD3D, and/or CD3G locus.

In some embodiments, the humanized CD3E locus lacks a human CD3E 5′-UTR. In some embodiment, the humanized CD3E locus comprises a rodent (e.g., mouse) 5′-UTR. In some embodiments, the humanization comprises a human 3′-UTR. In appropriate cases, it may be reasonable to presume that the mouse and human CD3E genes appear to be similarly regulated based on the similarity of their 5′-flanking sequence. As shown in the present disclosure, humanized CD3E mice that comprise a replacement at an endogenous mouse CD3E locus, which retain mouse regulatory elements but comprise a humanization of CD3E encoding sequence, do not exhibit obvious pathologies. Both genetically modified mice that are heterozygous or homozygous for humanized CD3E are grossly normal.

In some embodiments, the humanized CD3D locus has a human CD3D 5′ UTR. In some embodiments, the humanization comprises a human CD3D 3′-UTR. In some embodiments, the humanized CD3G locus has a human CD3G 5′ UTR. In some embodiments, the humanization comprises a human CD3G 3′-UTR.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CD3E gene, a humanized CD3D gene, and/or a humanized CD3G gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CD3E, CD3D, and/or CD3G in the genome of the animal.

In some embodiments, the non-human mammal comprises the genetic construct as described herein (e.g., gene construct as shown in FIG. 5 or FIG. 25). In some embodiments, a non-human mammal expressing human or humanized CD3E, CD3D, and/or CD3G is provided. In some embodiments, the tissue-specific expression of human or humanized CD3E, CD3D, and/or CD3G proteins is provided.

In some embodiments, the expression of human or humanized CD3E, CD3D, and/or CD3G in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human CD3E, CD3D, and/or CD3G proteins can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized CD3E, CD3D, and/or CD3G proteins.

The present disclosure also provides a TCR-CD3 complex, comprising: 1) an endogenous, human or humanized CD3E protein; (2) an endogenous, human or humanized CD3D protein; and/or (3) an endogenous, human or humanized CD3G protein. In some embodiments, the TCR-CD3 complex is functional. In some embodiments, at least one of CD3E, CD3D, and CD3G is a human protein. In some embodiments, the TCR-CD3 complex includes an endogenous CD3E protein, a human or humanized CD3D protein, and a human or humanized CD3G protein. In some embodiments, the TCR-CD3 complex includes a human or humanized CD3E protein, an endogenous CD3D protein, and a human or humanized CD3G protein. In some embodiments, the TCR-CD3 complex includes a human or humanized CD3E protein, a human or humanized CD3D protein, and an endogenous CD3G protein. In some embodiments, the TCR-CD3 complex includes an endogenous CD3E protein, an endogenous CD3D protein, and a human or humanized CD3G protein. In some embodiments, the TCR-CD3 complex includes an endogenous CD3E protein, a human or humanized CD3D protein, and an endogenous CD3G protein. In some embodiments, the TCR-CD3 complex includes a human or humanized CD3E protein, an endogenous CD3D protein, and an endogenous CD3G protein.

The present disclosure also provides a humanized CD3E mouse amino acid sequence, wherein the amino acid sequence is selected from the group consisting of:
a) an amino acid sequence shown in SEQ ID NO: 2 or SEQ ID NO: 66;
b) an amino acid sequence having a homology of at least 90% with or at least 90% identical to the amino acid sequence shown in SEQ ID NO: 2 or SEQ ID NO: 66;
c) an amino acid sequence encoded by a nucleic acid sequence, wherein the nucleic acid sequence is able to hybridize to a nucleotide sequence encoding the amino acid shown in SEQ ID NO: 2 or SEQ ID NO: 66 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2 or SEQ ID NO: 66;
e) an amino acid sequence that is different from the amino acid sequence shown in SEQ ID NO: 2 or SEQ ID NO: 66 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 2 or SEQ ID NO: 66.

The present disclosure also provides a humanized CD3D mouse amino acid sequence, wherein the amino acid sequence is selected from the group consisting of:
a) an amino acid sequence shown in SEQ ID NO: 4;
b) an amino acid sequence having a homology of at least 90% with or at least 90% identical to the amino acid sequence shown in SEQ ID NO: 4;
c) an amino acid sequence encoded by a nucleic acid sequence, wherein the nucleic acid sequence is able to hybridize to a nucleotide sequence encoding the amino acid shown in SEQ ID NO: 4 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 4;
e) an amino acid sequence that is different from the amino acid sequence shown in SEQ ID NO: 4 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 4.

The present disclosure also provides a humanized CD3G mouse amino acid sequence, wherein the amino acid sequence is selected from the group consisting of:
a) an amino acid sequence shown in SEQ ID NO: 6;
b) an amino acid sequence having a homology of at least 90% with or at least 90% identical to the amino acid sequence shown in SEQ ID NO: 6;
c) an amino acid sequence encoded by a nucleic acid sequence, wherein the nucleic acid sequence is able to hybridize to a nucleotide sequence encoding the amino acid shown in SEQ ID NO: 6 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 6;
e) an amino acid sequence that is different from the amino acid sequence shown in SEQ ID NO: 6 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 6.

The present disclosure also provides a humanized CD3E gene sequence, wherein the nucleic acid sequence can be selected from the group consisting of:
a) a nucleic acid sequence that is shown in SEQ ID NO: 14 or 63;
b) a nucleic acid sequence that has a homology of at least 75%, 80%, 85%, 90%, 91%, 92%, 93%, 94%, 95%, 96%, 97%, 98%, or 99% identical to SEQ ID NO: 14 or 63;
c) a nucleic acid sequence wherein the nucleotide sequence is different from the sequence shown in SEQ ID NO: 14 or 63 by no more than 10, 9, 8, 7, 6, 5, 4, 3, 2 or no more than 1 nucleotides; and/or
d) a nucleic acid sequence wherein the nucleotide sequence comprises a substitution, a deletion and/or insertion of one or more nucleotides to SEQ ID NO: 14 or 63.

The present disclosure also provides a humanized CD3E gene sequence, wherein the mRNA sequence transcribed therefrom is selected from the group consisting of:
a) a nucleic acid sequence that is shown in SEQ ID NO: 18 or 65;
b) a nucleic acid sequence that has a homology of at least 75%, 80%, 85%, 90%, 91%, 92%, 93%, 94%, 95%, 96%, 97%, 98%, or 99% identical to SEQ ID NO: 18 or 65;
c) a nucleic acid sequence wherein the nucleotide sequence is different from the sequence shown in SEQ ID NO: 18 or 65 by no more than 10, 9, 8, 7, 6, 5, 4, 3, 2 or no more than 1 nucleotides; and/or
d) a nucleic acid sequence wherein the nucleotide sequence comprises a substitution, a deletion and/or insertion of one or more nucleotides to SEQ ID NO: 18 or 65.

The present disclosure also provides a chimeric CD3D/CD3G gene, wherein the DNA sequence can be selected from the group consisting of:
a) a nucleic acid sequence that is shown in SEQ ID NO: 9;
b) a nucleic acid sequence that has a homology of at least 75%, 80%, 85%, 90%, 91%, 92%, 93%, 94%, 95%, 96%, 97%, 98%, or 99% identical to SEQ ID NO: 9;
c) a nucleic acid sequence wherein the nucleotide sequence is different from the sequence shown in SEQ ID NO: 9 by no more than 10, 9, 8, 7, 6, 5, 4, 3, 2 or no more than 1 nucleotides; and/or
d) a nucleic acid sequence wherein the nucleotide sequence comprises a substitution, a deletion and/or insertion of one or more nucleotides to SEQ ID NO: 9.

The present disclosure also relates to a nucleic acid (e.g., DNA or RNA) sequence, wherein the nucleic acid sequence can be selected from the group consisting of:
a) a nucleic acid sequence encoding a homologous CD3E, CD3D, or CD3G amino acid sequence of a humanized mouse;
b) a nucleic acid sequence that is shown in SEQ ID NO: 7, 8, 9, 10, 11, 12, 13, 14, 15, 16, 17, 18, 35, 36, 63, 64, or 65;
c) a nucleic acid sequence that is able to hybridize to the nucleotide sequence as shown in SEQ ID NO: 7, 8, 9, 10, 11, 12, 13, 14, 15, 16, 17, 18, 35, 36, 63, 64, or 65 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 7, 8, 9, 10, 11, 12, 13, 14, 15, 16, 17, 18, 35, 36, 63, 64, or 65;
e) a nucleic acid sequence that encodes an amino acid sequence, wherein the amino acid sequence has a homology of at least 90% with or at least 90% identical to the amino acid sequence shown in SEQ ID NO: 2, 4, 6, or 66;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2, 4, 6, or 66;
g) a nucleic acid sequence that encodes an amino acid sequence, wherein the amino acid sequence is different from the amino acid sequence shown in SEQ ID NO: 2, 4, 6, or 66 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and/or insertion of one or more amino acids to the amino acid sequence shown in SEQ ID NO: 2, 4, 6, or 66.

The present disclosure further relates to a CD3E, CD3D, and/or CD3G genomic DNA sequence of a humanized mouse. The DNA sequence can be obtained by a reverse transcription of the mRNA obtained by transcription thereof. In some embodiments, the DNA sequence is consistent with or complementary to a DNA sequence homologous to the sequence shown in SEQ ID NO: 9, SEQ ID NO: 14, SEQ ID NO: 18, SEQ ID NO: 63, or SEQ ID NO: 65.

The disclosure also provides an amino acid sequence that has a homology of at least 90% with, or at least 90% identical to the sequence shown in SEQ ID NO: 2, 4, 6, or 66, and has protein activity. In some embodiments, the homology with the sequence shown in 2, 4, 6, or 66 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in 2, 4, 6, or 66 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 9, SEQ ID NO: 14, SEQ ID NO: 18, SEQ ID NO: 63, or SEQ ID NO: 65, and encodes a polypeptide that has protein activity. In some embodiments, the homology with the sequence shown in SEQ ID NO: 9, SEQ ID NO: 14, SEQ ID NO: 18, SEQ ID NO: 63, or SEQ ID NO: 65 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

In some embodiments, provided herein is a genetically-modified non-human animal comprising any of the amino acid sequences described herein and/or any of the nucleic acid sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For illustration purposes, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g., leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CD3E, CD3D, and/or CD3G from an endogenous non-human CD3E, CD3D, and/or CD3G locus.

### Vectors

The present disclosure relates to a targeting vector, comprising: a) a DNA fragment homologous to the 5′ end of a region to be altered (5′ arm), which is selected from the CD3E gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CD3E gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000075.7; c) the DNA fragment homologous to the 3′ end of the region to be altered (3′ arm) is selected from the nucleotide sequences that have at least 90% homology to the NCBI accession number NC_000075.7.

In some embodiments, a) the DNA fragment homologous to the 5′ end of a region to be altered (5′ arm) is selected from the nucleotides from the position 44898911 to the position 44903464 of the NCBI accession number NC_000075.7; c) the DNA fragment homologous to the 3′ end of the region to be altered (3′ arm) is selected from the nucleotides from the position 44875592 to the position 44880865 of the NCBI accession number NC_000075.7. In some embodiments, a) the DNA fragment homologous to the 5′ end of a region to be altered (5′ arm) is selected from the nucleotides from the position 44920695 to the position 44925940 of the NCBI accession number NC_000075.7; c) the DNA fragment homologous to the 3′ end of the region to be altered (3′ arm) is selected from the nucleotides from the position 44906192 to the position 44909738 of the NCBI accession number NC_000075.7.

In some embodiments, the length of the selected genomic nucleotide sequence in the targeting vector can be more than about 10 kb, about 11 kb, about 12 kb, about 13 kb, about 14 kb, about 15 kb, about 16 kb, or about 17 kb.

In some embodiments, the region to be altered is exon 1, exon 2, exon 3, exon 4, exon 5, exon 6, exon 7, and/or exon8 of CD3E gene (e.g., exon 2, exon 3, exon 4, exon 5, and exon 6 of mouse CD3E gene). In some embodiments, the region to be altered is exon 1, exon 2, exon 3, exon 4, and/or exon 5 of CD3D gene (e.g., exon 1, exon 2, exon 3, exon 4, and exon 5 of mouse CD3D gene). In some embodiments, the region to be altered is exon 1, exon 2, exon 3, exon 4, exon 5, exon 6, and/or exon 7 of CD3G gene (e.g., exon 1, exon 2, exon 3, exon 4, exon 5, exon 6, and exon 7 of mouse CD3G gene).

The targeting vector can further include a selected gene marker (e.g., HygR, Neo, and/or DTA).

In some embodiments, the sequence of the 5′ arm is shown in SEQ ID NO: 7; and the sequence of the 3′ arm is shown in SEQ ID NO: 8. In some embodiments, the sequence of the 5′ arm is shown in SEQ ID NO: 12. In some embodiments, the sequence of the 3′ arm is shown in SEQ ID NO: 13.

In some embodiments, the sequence is derived from human CD3E gene (e.g., 118304953-118315542 of NC_000011.10 (SEQ ID NO: 14) or 118304953-118313732 of NC_000011.10 (SEQ ID NO: 63)). For example, the target region in the targeting vector is a part or entirety of the nucleotide sequence of a human CD3E, preferably exon 2, exon 3, exon 4, exon 5, exon 6, and/or exon 7 of the human CD3E. In some embodiments, the nucleotide sequence of the humanized CD3E encodes the entire or the part of human CD3E protein with the NCBI accession number NP_000724.1 (SEQ ID NO: 2). In some embodiments, the sequence is derived from human CD3D and CD3G genes (e.g., 118338117-118355186 of NC_000011.10 (SEQ ID NO: 9)). For example, the target region in the targeting vector is a part or entirety of the nucleotide sequence of a human CD3D (preferably exon 1, exon 2, exon 3, exon 4, and/or exon 5 of the human CD3D) and a part or entirety of the nucleotide sequence of a human CD3G (preferably exon 1, exon 2, exon 3, exon 4, exon 5, exon 6, and/or exon 7 of the human CD3G). In some embodiments, the nucleotide sequence of the humanized CD3D encodes the entire or the part of human CD3D protein with the NCBI accession number NP_000723.1 (SEQ ID NO: 4). In some embodiments, the nucleotide sequence of the humanized CD3G encodes the entire or the part of human CD3G protein with the NCBI accession number NP_000064.1 (SEQ ID NO: 6).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous CD3E gene locus, a sequence encoding a region of an endogenous CD3E with a sequence encoding a corresponding region of human or chimeric CD3E. In some embodiments, the disclosure provides replacing in at least one cell of the animal, at endogenous CD3D and CD3G gene loci, a sequence encoding a region of an endogenous CD3D and CD3G with a sequence encoding a corresponding region of human or chimeric CD3D and CD3G. In some embodiments, the replacement occurs in a germ cell, a somatic cell, a blastocyst, or a fibroblast, etc. The nucleus of a somatic cell or the fibroblast can be inserted into an enucleated oocyte.

FIG. 5 and FIG. 25 show humanization strategies for mouse CD3E, CD3D, and CD3G loci. In FIG. 5 and FIG. 25, the targeting strategies involve a first vector comprising the 5′ end homologous arm, human CD3D/CD3G gene fragment, 3′ homologous arm; and a second vector comprising the 5′ end homologous arm, human CD3E gene fragment, 3′ homologous arm. The process can involve replacing endogenous CD3D/CD3G sequence with human sequence, then replacing endogenous CD3E sequence with human sequence, by homologous recombination. In some embodiments, the cleavage at the upstream and the downstream of the target site (e.g., by zinc finger nucleases, TALEN or CRISPR) can result in DNA double strands break, and the homologous recombination is used to replace endogenous CD3E, CD3D, and CD3G sequences with human CD3E, CD3D, and CD3G sequences.

Thus, in some embodiments, the methods for making a genetically modified, humanized animal, can include the step of replacing at an endogenous CD3E locus (or site), a nucleic acid encoding a sequence encoding a region of endogenous CD3E with a sequence encoding a corresponding region of human CD3E. The sequence can include a region (e.g., a part or the entire region) of exon 1, exon 2, exon 3, exon 4, exon 5, exon 6, exon 7, exon 8, and/or exon 9 of a human CD3E gene. In some embodiments, the sequence includes a region of exon 2, exon 3, exon 4, exon 5, exon 6, and exon 7 of a human CD3E gene (e.g., amino acids 1-126 of SEQ ID NO: 7). In some embodiments, the sequence includes a region of exon 2, exon 3, exon 4, exon 5, exon 6, exon 7, exon 8, and exon 9 of a human CD3E gene. In some embodiments, the region is located within the extracellular region of CD3E. In some embodiments, the endogenous CD3E locus is exon 2, exon 3, exon 4, exon 5, and/or exon 6 of mouse CD3E.

In some embodiments, the methods for making a genetically modified, humanized animal, can include the step of replacing at endogenous CD3D and CD3G loci (or sites), a nucleic acid encoding a sequence encoding endogenous CD3D and CD3G with a sequence encoding human CD3D and CD3G. The sequence can include a region (e.g., a part or the entire region) of exon 1, exon 2, exon 3, exon 4, and/or exon 5 of a human CD3D gene; and a region (e.g., a part or the entire region) of exon 1, exon 2, exon 3, exon 4, exon 5, exon 6, and/or exon 7 of a human CD3G gene. In some embodiments, the endogenous CD3D locus is exon 1, exon 2, exon 3, exon 4, and/or exon 5 of mouse CD3D. In some embodiments, the endogenous CD3G locus is exon 1, exon 2, exon 3, exon 4, exon 5, exon 6, and/or exon 7 of mouse CD3G.

In some embodiments, the methods of modifying a CD3E locus of a mouse to express a chimeric human/mouse CD3E peptide can include the steps of replacing at the endogenous mouse CD3E locus a nucleotide sequence encoding a mouse CD3E with a nucleotide sequence encoding a human CD3E, thereby generating a sequence encoding a chimeric human/mouse CD3E. In some embodiments, the methods of modifying a CD3D locus of a mouse to express a chimeric human/mouse CD3D peptide can include the steps of replacing at the endogenous mouse CD3D locus a nucleotide sequence encoding a mouse CD3D with a nucleotide sequence encoding a human CD3D, thereby generating a sequence encoding a chimeric human/mouse CD3D. In some embodiments, the methods of modifying a CD3G locus of a mouse to express a chimeric human/mouse CD3G peptide can include the steps of replacing at the endogenous mouse CD3G locus a nucleotide sequence encoding a mouse CD3G with a nucleotide sequence encoding a human CD3G, thereby generating a sequence encoding a chimeric human/mouse CD3G.

In some embodiments, the nucleotide sequence encoding the chimeric human/mouse CD3E can include a first nucleotide sequence encoding an extracellular region of mouse CD3E (with or without the mouse or human signal peptide sequence); a second nucleotide sequence encoding an extracellular region of human CD3E; a third nucleotide sequence encoding a transmembrane and a cytoplasmic region of a mouse CD3E.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a CD3E, CD3D, and/or CD3G gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal; and
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

Also provided herein is a method for making a genetically-modified non-human animal, comprising: a) inserting or replacing at an endogenous CD3E gene locus, a nucleotide sequence comprising a sequence that is at least 90% identical to comprising SEQ ID NO: 14 or 63; and b) insertion or replacing at endogenous CD3D and CD3G gene loci, a nucleotide sequence comprising a sequence that is at least 90% identical to comprising SEQ ID NO: 9. In some embodiments, step b) is performed prior to step a).

The present disclosure also relates to genetic modification of endogenous CD3E, CD3D, and/or CD3G genes, and the number or order of the modification in the non-human animal genome is not limited. In some embodiments, each gene can be modified individually. For example, the CD3E, CD3D, CD3G genes can be modified sequentially; the CD3D, CD3G, CD3E genes can be modified sequentially; the CD3G, CD3E, CD3D genes can be modified sequentially; the CD3E, CD3G, CD3D genes can be modified sequentially; the CD3D, CD3E, CD3G genes can be modified sequentially; or the CD3G, CD3D, CD3E genes can be modified sequentially. In some embodiments, one gene is modified first and then the other two genes are modified subsequently. In some embodiments, two genes are modified first and then the other gene is modified subsequently. For example, the CD3E gene is modified followed by the CD3G and CD3D genes; the CD3D gene is modified followed by the CD3E and CD3G genes; or the CD3G gene is modified followed by the CD3E and CD3D genes. Alternatively, all three genes can be modified simultaneously.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized CD3E, CD3D, and/or CD3G proteins, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CD3E, CD3D, and/or CD3G, which are useful for testing agents that can decrease or block the interaction between T cell receptor and/or co-receptor with its ligands, testing the interaction between CD3E (or CD3) and anti-human CD3E antibodies, testing the interaction between CD3D (or CD3) and anti-human CD3D antibodies, testing the interaction between CD3G (or CD3) and anti-human CD3G antibodies, testing whether an agent can increase or decrease the immune response, and/or determining whether an agent is an CD3 (or T cell receptor and/or co-receptor) agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of an anti-CD3E antibody, an anti-CD3D antibody, or an anti-CD3G antibody for the treatment of cancer. The methods involve administering the anti-CD3E antibody (e.g., anti-human CD3E antibody), the anti-CD3D antibody (e.g., anti-human CD3D antibody), or the anti-CD3G antibody (e.g., anti-human CD3G antibody) to the animal as described herein, wherein the animal has a tumor; and determining the inhibitory effects of the anti-CD3E antibody, the anti-CD3D antibody, or the anti-CD3G antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the anti-CD3E antibody, the anti-CD3D antibody, or the anti-CD3G antibody prevents antigens presented by MHC from binding to T cell receptors (TCR). In some embodiments, the anti-CD3E antibody, the anti-CD3D antibody, or the anti-CD3G antibody does not prevent antigens presented by MHC from binding to T cell receptors.

In some embodiments, the genetically modified animals can be used for determining whether an anti-CD3E antibody, an anti-CD3D antibody, or an anti-CD3G antibody is a CD3 (or TCR) agonist or antagonist. In some embodiments, the methods as described herein are also designed to determine the effects of the agent (e.g., anti-CD3E, anti-CD3D, or anti-CD3G antibodies) on CD3 (or TCR), e.g., whether the agent can stimulate immune cells or inhibit immune cells (e.g., T cells), whether the agent can increase or decrease the production of cytokines, whether the agent can activate or deactivate immune cells (e.g., T cells), whether the agent can upregulate the immune response or downregulate immune response, whether the agent can cause activation induced cell death (AICD), and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancers, autoimmune diseases, or inflammation.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups. In some embodiments, the TGI_{TV}% value is negative, which means that the tested agent decreases immune response, and/or promotes tumor growth.

In some embodiments, the anti-CD3E antibody, anti-CD3D antibody, anti-CD3G antibody, or a multi-specific antibody (e.g., a bispecific antibody) targeting CD3 is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-CD3E antibody, the anti-CD3D antibody, or the anti-CD3G antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-CD3E antibody, the anti-CD3D antibody, or the anti-CD3G antibody is designed for treating hepatocellular, ovarian, colon, or cervical carcinomas. In some embodiments, the anti-CD3E antibody, the anti-CD3D antibody, or the anti-CD3G antibody is designed for treating advanced breast cancer, advanced ovarian cancer, and/or advanced refractory solid tumor. In some embodiments, the anti-CD3E antibody, the anti-CD3D antibody, or the anti-CD3G antibody is designed for treating metastatic solid tumors, NSCLC, melanoma, non-Hodgkin lymphoma, colorectal cancer, and multiple myeloma. In some embodiments, the anti-CD3E antibody, the anti-CD3D antibody, or the anti-CD3G antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-CD3E antibody, the anti-CD3D antibody, or the anti-CD3G antibody is designed for treating carcinomas (e.g., nasopharynx carcinoma, bladder carcinoma, cervix carcinoma, kidney carcinoma or ovary carcinoma).

In some embodiments, the anti-CD3E antibody, the anti-CD3D antibody, or the anti-CD3G antibody is designed for treating, preventing, or reducing the risk of developing disorders associated with an abnormal or unwanted immune response, e.g., an autoimmune disorder, e.g., by affecting the functional properties of the circulating CD3+ T cells (e.g., reducing their proliferative capacity) or by inducing regulatory cells. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-CD3E antibody, the anti-CD3D antibody, or the anti-CD3G antibody or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has Crohn's disease, ulcerative colitis or type 1 diabetes. Thus, the methods as described herein can be used to determine the effectiveness of an anti-CD3E antibody, anti-CD3D antibody, or anti-CD3G antibody in inhibiting immune response, and the animals can be used as models for testing agents for treating these autoimmune diseases.

In some embodiments, the cancer described herein is selected from lymphoma, B cell tumor, T cell tumor, bone marrow/monocyte tumor, non-small cell lung cancer, leukemia, ovarian cancer, nasopharyngeal cancer, breast cancer, endometrial cancer, colon cancer, rectal cancer, stomach cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, and sarcoma. Wherein, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom's macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In some embodiments, the cancer described herein is selected from B cell tumors, T cell tumors, and myeloid/monocyte tumors. In some embodiments, the cancer is selected from B or T cell acute lymphoblastic leukemia (ALL), acute myeloid leukemia (AML), non-Hodgkin lymphoma (NEIL), multiple myeloma (MM), nasopharyngeal carcinoma, and lung cancer.

The present disclosure also provides methods of determining toxicity of an antibody (e.g., an anti-CD3E antibody, an anti-CD3D antibody, or an anti-CD3G antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CD3E, CD3D, and CD3G gene function; anti-human CD3E, CD3D, and/or CD3G antibodies; drugs for human CD3E, CD3D, and/or CD3G targeting sites; the drugs or efficacies for human CD3E, CD3D, and/or CD3G targeting sites; and the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the CD3E, CD3D, and/or CD3G gene humanized non-human animals prepared by the methods described herein, the CD3E, CD3D, and/or CD3G gene humanized non-human animals described herein, the double- or multi-humanized non-human animal generated by the methods described herein (or progeny thereof), a non-human animal expressing the human or humanized CD3E, CD3D, and/or CD3G proteins, or the tumor-bearing or inflammatory animal models described herein. In some embodiments, the TCR-T, CAR-T, and/or other immunotherapies can treat the CD3-associated diseases described herein. In some embodiments, the TCR-T, CAR-T, and/or other immunotherapies provides an evaluation method for treating the CD3-associated diseases described herein.

Also provided herein is a screening method for human CD3E, CD3D and/or CD3G specific regulators. The screening method comprises applying a regulator to a subject, and detecting the regulation effect. In some embodiments, the subject is selected from any of the non-human animals described herein, any of the non-human animals obtained using any methods described herein, any of the disease models described herein, and progeny thereof. In some embodiments, the regulator is selected from CAR-T molecules, antibodies, or drugs. In some embodiments, the regulator is a monoclonal antibody, bispecific antibody, or a combination of two or more drugs. In some embodiments, the method includes injecting tumor cells to the subject.

Also provided herein is a method for activating T cells, comprising: administering a human CD3E, CD3D and/or CD3G specific regulator to the animal as described herein. In some embodiments, the regulator is a multi-specific antibody (e.g., a bispecific antibody). In some embodiments, the bispecific antibody targets CD3 and a tumor-associated antigen (TAA) or an infectious disease antigen. In some embodiments, the TAA is selected from ALK, BAGE protein, BIRC5 (survivin), BIRC7, CA9, CALR, CCR5, CD19, CD20 (MS4A1), CD22, CD27, CD30, CD33, CD38, CD40, CD44, CD52, CD56, CD79, CDK4, CEACAM3, CEACAM5, CLEC12A, EGFR, EGFR variant III, ERBB2 (HER2), ERBB3, ERBB4, EPCAM, EPHA2, EPHA3, FCRL5, FLT3, FOLR1, GAGE proteins, GD2, GD3, GPNMB, GM3, GPR112, IL3RA, KIT, KRAS, LGR5, EBV-derived LMP2, L1 CAM, MAGE protein, MLANA, MSLN, MUC1, MUC2, MUC3, MUC4, MUC5, MUC16, MUM1, ANKRD30A, NY-ESO1 (CTAG1B), OX40, PAP, PAX3, PAX5, PLAC1, PRLR, PMEL, PRAME, PSMA (FOLH1), RAGE protein, RET, RGS5, ROR1, SART1, SART3, SLAMF7, SLC39A6 (LIV1), STEAP1, S TERT, TMPRS S2, Thompson-nouvelle antigen, TNFRSF17, TYR, UPK3A, VTCN1 and WT1. In some embodiments, the infectious disease antigen is a virus antigen or bacterial antigen. In some embodiments, the virus antigen is selected from HIV, Hepatitis A, Hepatitis B, Hepatitis C, Herpes Viruses e.g. HSV-1, HSV-2, CMV, HAV-6, VZV, Epstein-Barr virus, Adenovirus, Influenza virus, Yellow fever virus, Echo virus, Rhinovirus, coxsackie virus, coronavirus, respiratory syncytial virus, mumps virus, rotavirus, measles virus, rubella virus, parvovirus, vaccinia virus, HTLV, dengue virus, papilloma virus, molluscum virus, spinal cord Polio virus, rabies virus, JC virus, Ebola virus and arbovirus encephalitis. In some embodiments, the bacterial antigen is selected from chlamydia, rickettsia, mycobacteria, staphylococcus, streptococcus, pneumococcus, meningococcus, gonococcus, klebsiella, proteus, proteus, Serratia, Pseudomonas, Legionella, Diphtheria, Salmonella, Bacillus, Cholera, Tetanus, Botulinum, Bacillus anthracis, Plague, Leptospira and Lyme Disease.

Genetically Modified Animal Model with Two or More Human or Chimeric Genes

The present disclosure further relates to methods for generating genetically modified animal model with two or more human or chimeric genes. The animal can comprise a human or chimeric CD3E, CD3D, and/or CD3G gene and a sequence encoding an additional human or chimeric protein. In some embodiments, the animal comprises a sequence encoding an endogenous CD3-zeta protein.

In some embodiments, the additional human or chimeric protein can be programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), TNF Receptor Superfamily Member 4 (TNFRSF4 or OX40), CD40, Inducible T-cell COStimulator (ICOS or CD278) or Signal regulatory protein a (SIRPa).

The methods of generating genetically modified animal model with two or more human or chimeric genes (e.g., humanized genes) can include the following steps:
(a) using the methods of introducing human CD3EDG gene or chimeric CD3EDG gene as described herein to obtain a genetically modified non-human animal;
(b) breeding the genetically modified non-human animal with another genetically modified non-human animal, and then screening the progeny to obtain a genetically modified non-human animal with two or more human or chimeric genes.

In some embodiments, the breeding step can be substituted by in vitro fertilization or gene editing.

In some embodiments, in step (b) of the method, the genetically modified animal can be mated with a genetically modified non-human animal with human or chimeric PD-1, CTLA-4, LAG-3, BTLA, PD-L1, CD27, CD28, CD38, CD47, CD137, CD154, TIGIT, TIM-3, GITR, SIRPa, OX40, CD40, B7-H3, CLDN18.2, or CD278. Some of these genetically modified non-human animals are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the CD3E, CD3D, and/or CD3G gene humanization is directly performed on a genetically modified animal having a human or chimeric PD-1, CTLA-4, LAG-3, BTLA, PD-L1, CD27, CD28, CD38, CD47, CD137, CD154, TIGIT, TIM-3, GITR, SIRPa, OX40, CD40, B7-H3, CLDN18.2, or CD278 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CD3E antibody (or an anti-CD3D antibody, an anti-CD3G antibody) and an additional therapeutic agent for the treatment of cancer. The methods include administering the anti-CD3E antibody (or the anti-CD3D antibody, the anti-CD3G antibody) and the additional therapeutic agent to the animal, wherein the animal has a tumor; and determining the inhibitory effects of the combined treatment to the tumor. In some embodiments, the additional therapeutic agent is an antibody that specifically binds to PD-1, CTLA-4, BTLA, PD-L1, CD27, CD28, CD47, CD137, CD154, TIGIT, TIM-3, GITR, OX40, CD40, SIRPa or CD278. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various autoimmune diseases as described herein.

In some embodiments, the autoimmune disease described herein is selected from allergy, asthma, myocarditis, nephritis, hepatitis, systemic lupus erythematosus, rheumatoid arthritis, scleroderma, hyperthyroidism, idiopathic thrombocytopenic purpura, autoimmune hemolytic anemia, ulcerative colitis, autologous Immune liver disease, diabetes, pain or neurological disorders. In some embodiments, the inflammation described herein is selected from chronic inflammation, degenerative inflammation, exudative inflammation (e.g., serous inflammation, fibrinitis, suppurative inflammation, hemorrhagic inflammation, necrotizing inflammation, or catarrhal inflammation), proliferative inflammation, specific inflammation (tuberculosis, syphilis, leprosy, or lymphogranuloma).

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

C57BL/6 mice and F1p transgenic mice were purchased from the China Food and Drugs Research Institute National Rodent Experimental Animal Center.

Heraeus™ Fresco™ 21 Microcentrifuge was purchased from Thermo Fisher Scientific (model: Fresco™ 21).

Attune™ NxT Acoustic Focusing Cytometer was purchased from Thermo Fisher (model: Attune™ NxT).

PrimeScript™ 1st Strand cDNA Synthesis Kit was purchased from Takara Bio (Catalog number: 6110A).

NdeI, EcoRI, SspI, XbaI, SpeI, and EcoRV restriction enzymes were purchased from NEB (Catalog numbers: R0111S, R0101M, R0132M, R0145M, R0133M, and R0195M).

Purified anti-mouse CD16/32 Antibody was purchased from BioLegend (Catalog number: 101302).

Zombie NIR™ Fixable Viability Kit (DMSO) was purchased from BioLegend (Catalog number: 423106).

Foxp3/Transcription Factor Staining Buffer Set was purchased from eBioscience (Catalog number: 00-5523-00).

PerCP anti-mouse CD45 Antibody was purchased from BioLegend (Catalog number: 103130).

Brilliant Violet 711™ anti-mouse TCRβ Chain Antibody was purchased from BioLegend (Catalog number: 109243).

Brilliant Violet 510™ anti-mouse CD4 Antibody was purchased from BioLegend (Catalog number: 100449).

FITC anti-Mouse CD8a Antibody was purchased from BioLegend (Catalog number: 100706).

Brilliant Violet 421™ anti-mouse NK1.1 Antibody was purchased from BioLegend (Catalog number: 108732).

Brilliant Violet 605™ anti-mouse CD19 Antibody was purchased from BioLegend (Catalog number: 115540).

Anti-Mo/Rt Foxp3PE/Cy™ 7 was purchased from eBioscience (Catalog number: 25-5773-82).

Brilliant Violet 510™ anti-mouse CD45 Antibody was purchased from BioLegend (Catalog number: 103138).

PerCP anti-mouse Ly-6G/Ly-6C (Gr-1) Antibody was purchased from BioLegend (Catalog number: 108426).

Anti-mouse CD11c PE/Cy7 Antibody was purchased from eBioscience (Catalog number: 25-0114-81).

V450 Rat Anti-mouse CD11b was purchased from BD Horizon (Catalog number: 560455).

FITC anti-mouse F4/80 Antibody was purchased from BioLegend (Catalog number: 123108).

SBA Clonotyping System-057BL/6-HRP was purchased from SouthernBiotech (Catalog number: 5300-05B).

Goat Anti-mouse IgG Fc-HRP was purchased from Abcam (Catalog number: ab98741).

Mouse IgG1, kappa Isotype Control was purchased from CrownBio (Catalog number: C0005).

Mouse IgG2b, kappa Isotype Control was purchased from CrownBio (Catalog number: C0008).

### Example 1: Preparation of Humanized Mice with CD3EDG Gene

In this example, a non-human animal (e.g., a mouse) was modified to include a nucleotide sequence encoding human CD3E, CD3D, and CD3G proteins, and the obtained genetically-modified non-human animal can express human or humanized CD3E, CD3D, and CD3G proteins in vivo. The mouse CD3E gene (NCBI Gene ID: 12501, Primary source: MGI: 88332, UniProt ID: P22646) is located at 44910033 to 44920961 of chromosome 9 (NC_000075.7), and the human CD3E gene (NCBI Gene ID: 916, Primary source: HGNC: 1674, UniProt ID: P07766) is located at 118304730 to 118316173 of chromosome 11 (NC_000011.10). The mouse CD3E transcript is NM_007648.5, and the corresponding protein sequence NP_031674.1 is set forth in SEQ ID NO: 1. The human CD3E transcript is NM_000733.4, and the corresponding protein sequence NP_000724.1 is set forth in SEQ ID NO: 2. Mouse and human CD3E gene loci are shown in FIG. 1.

The mouse CD3D gene (NCBI Gene ID: 12500, Primary source: MGI: 88331, UniProt ID: P04235) is located at 44893067 to 44898350 of chromosome 9 (NC_000075.7), and the human CD3D gene (NCBI Gene ID: 915, Primary source: HGNC: 1673, UniProt ID: P04234) is located at 118338954 to 118342705 of chromosome 11 (NC_000011.10). The mouse CD3D transcript is NM_013487.3, and the corresponding protein sequence NP_038515.3 is set forth in SEQ ID NO: 3. The human CD3D transcript is NM_000732.6, and the corresponding protein sequence NP_000723.1 is set forth in SEQ ID NO: 4. Mouse and human CD3D gene loci are shown in FIG. 2.

The mouse CD3G gene (NCBI Gene ID: 12502, Primary source: MGI: 88333, UniProt ID: P11942) is located at 44880870 to 44891729 of chromosome 9 (NC_000075.7), and the human CD3G gene (NCBI Gene ID: 917, Primary source: HGNC: 1675, UniProt ID: P09693) is located at 118344344 to 118355161 of chromosome 11 (NC_000011.10). The mouse CD3G transcript is NM_009850.2, and the corresponding protein sequence NP_033980.1 is set forth in SEQ ID NO: 5. The human CD3G transcript is NM_000073.3, and the corresponding protein sequence NP_000064.1 is set forth in SEQ ID NO: 6. Mouse and human CD3G gene loci are shown in FIG. 3.

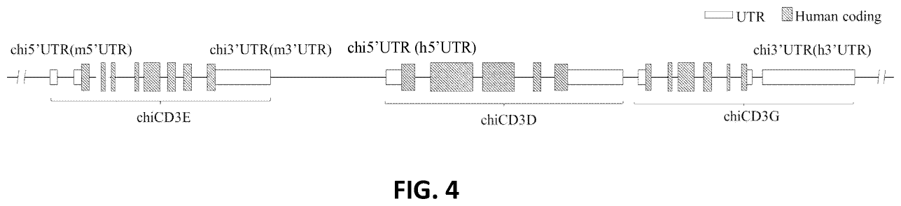

All or part of nucleotide sequences encoding human CD3E, CD3D and CD3G proteins can be introduced into the mouse endogenous CD3E, CD3D and CD3G loci, so that the mouse expresses human or humanized CD3E, CD3D and CD3G proteins. Mouse cells can be modified by various gene-editing techniques, for example, replacement of specific mouse CD3E gene sequences with human CD3E gene sequences at the endogenous mouse CD3E locus. For example, a sequence (about 9.8 kb) spanning from exon 2 (including a part of exon 2) to exon 8 (including a part of exon 8) of mouse CD3E gene can be replaced with a sequence (about 11 kb) spanning from exon 2 (including a part of exon 2) to exon 9 (including a part of exon 9) of human CD3E gene, and the mouse CD3D and CD3G loci can be replaced with a 17 kb DNA sequence from human CD3D and CD3G loci, to obtain a humanized mouse CD3E, CD3D, and CD3G loci as shown in FIG. 4, thereby humanizing mouse CD3E, CD3D, and CD3G genes.

As shown in the schematic diagram of the targeting strategy in FIG. 5, targeting vector 1 (V1) and targeting vector 2 (V2) were involved. The targeting vector 1 contains homologous arm sequences upstream of the mouse CD3D gene and downstream of the mouse CD3G gene, and a fragment A1 containing DNA sequences of human CD3D and CD3G genes. Specifically, sequence of the upstream homologous arm (5′ homologous arm 1, SEQ ID NO: 7) is identical to nucleotide sequence of 44898911-44903464 of NCBI accession number NC_000075.7, and sequence of the downstream homologous arm (3′ homologous arm 1, SEQ ID NO: 8) is identical to nucleotide sequence of 44875592-44880865 of NCBI accession number NC_000075.7. The fragment A1 contains a human genomic DNA sequence from CD3D and CD3G genes (SEQ ID NO: 9), which is identical to nucleotide sequence of 118338117-118355186 of NCBI accession number NC_000011.10. This sequence is directly connected to the 5′ homologous arm 1, and the connection was designed as:

(SEQ ID NO: 35)
5′-TGGTACGTGCTTGTAATCCTAACACTGTGAGATATCGTGGCTCATG
CCTCTAATCCCAGCACTTTTGGAG-3′,

wherein the “A” in sequence “TGTGA” is the last nucleotide of the 5′ homologous arm 1, and the first “G” in sequence “GTGGC” is the first nucleotide of the human sequence. The fragment A1 also includes an antibiotic resistance gene for positive clone screening (Hygromycin resistance gene, or HygR), and two Frt recombination sites flanking the antibiotic resistance gene, that formed a HygR cassette. The connection between the 5′ end of the HygR cassette and the human CD3D/CD3G DNA sequence was designed as:

(SEQ ID NO: 10)
5′-CACAAATAAATTGAATTGTTGGGCACTTGTATTTGCATTTGTAATT
TTATAAGCTTGATATCATAACTTCGTATAATGTATGCTATACGAAGTTA
TAGGT-3′,

wherein the last “T” in sequence “TTTAT” is the last nucleotide of the human sequence, and the first “A” in sequence “AAGCT” is the first nucleotide of the HygR cassette. The connection “ ” between the 3′ end of the HygR cassette and the 3′ homologous arm 1 was

(SEQ ID NO: 11)
5′-CTTCATAACTTCGTATAATGTATGCTATACGAAGTTATGGATCCGA
GCTCTCTATCCAAATAGACATTTCTTTTTTTAATGTTTGCTGGGTTCTT
ACGCA-3′,

wherein the last “C” in sequence “AGCTC” is the last nucleotide of the HygR cassette, and the first “T” in sequence “TCTAT” is the first nucleotide of the 3′ homologous arm 1. In addition, a coding gene with a negative selectable marker (a gene encoding diphtheria toxin A subunit (DTA)) was also constructed downstream of the 3′ homologous arm of the targeting vector 1.

The targeting vector 2 contains homologous arm sequences upstream and downstream of the mouse CD3E gene, and a fragment A2 containing the DNA sequences of human CD3E gene. Specifically, sequence of the upstream homologous arm (5′ homologous arm 2, SEQ ID NO: 12) is identical to nucleotide sequence of 44920695-44925940 of NCBI accession number NC_000075.7, and sequence of the downstream homologous arm (3′ homologous arm 2, SEQ ID NO: 13) is identical to nucleotide sequence of 44906192-44909738 of NCBI accession number NC_000075.7. The fragment A2 contains a human CD3E genomic DNA sequence (SEQ ID NO: 14), which is identical to nucleotide sequence of 118304953-118315542 of NCBI accession number NC_000011.10. The connection between the 5′ end of the human CD3E DNA sequence and the mouse sequence was designed as:

(SEQ ID NO: 36)
5′-GCTGCGTCCGCCATCTTGGTAGAGAGAGCATTCTGAGAGGATGCAG
TCGGGCACTCACTGGAGAGTTCTGGG-3′,

wherein the last “G” in sequence “AGAGG” is the last nucleotide of the mouse sequence, and the first “A” in sequence “ATGCA” is the first nucleotide of the human sequence. The connection between the 3′ end of the human CD3E DNA sequence and the mouse sequence was designed as:

(SEQ ID NO: 15)
5′-GGAAAGGCCAGCGGGACCTGTATTCTGGCCTGAATCAGAGACGCAT
CTGACAGATAGGAGAGACATCGCCTTCTGTGGACCCAGATCCAGCCCTC
CGAGC-3′,

wherein the “A” in sequence “TCTGA” is the last nucleotide of the human sequence, and the “C” in sequence “CAGAT” is the first nucleotide of the mouse sequence. The fragment A2 also includes an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the mouse sequence was designed as:

(SEQ ID NO: 16)
5′-TGGTGAGGAAGGAATTGGGGCTGAGAAGCTGGTTTCCATCAGCTGC
CAGGAAGCTTGATATCGAATTCCGAAGTTCCTATTCTCTAGAAAGTATA
GGAAC-3′,

wherein the last “G” in sequence “CCAGG” is the last nucleotide of the mouse sequence, and the first “A” in sequence “AAGCT” is the first nucleotide of the Neo cassette. The connection between the 3′ end of the Neo cassette and the mouse sequence was designed as:

(SEQ ID NO: 17)
5′-AAAGTATAGGAACTTCATCAGTCAGGTACATAATGGTGGATCCAGA
ATTCAAGTGCTGCTGAACAGAGCCAGGGAAGACGAGAAAAGCCGTGCGT
GCGTG-3′,

wherein the “C” in sequence “AATTC” is the last nucleotide of the Neo cassette, and the first “A” in sequence “AAGTG” is the first nucleotide of the mouse sequence. In addition, a coding gene with a negative selectable marker (a gene encoding diphtheria toxin A subunit (DTA)) was also constructed downstream of the 3′ homologous arm of the targeting vector 2.

The mRNA sequence of the engineered mouse CD3E after humanization and its encoded protein sequence are shown in SEQ ID NO: 18 and SEQ ID NO: 2, respectively. The protein sequences expressed by humanized mouse CD3D and CD3G genes (hereinafter referred to as CD3DG) are shown in SEQ ID NO: 4 and SEQ ID NO: 6, respectively.

In a different example, a sequence spanning from exon 2 (including a part of exon 2) to exon 6 (including a part of exon 6) of mouse CD3E can be replaced with a sequence spanning from exon 2 (including a part of exon 2) to exon 7 (including a part of exon 7) of human CD3E gene, and the mouse CD3D and CD3E loci can be replaced with a 17 kb DNA sequence from human CD3D and CD3G loci, to obtain a humanized mouse CD3E, CD3D, and CD3G loci as shown in FIG. 25, thereby humanizing mouse CD3E, CD3D, and CD3G genes.

As shown in the schematic diagram of the targeting strategy in FIG. 25, targeting vector 1 (V1) and targeting vector 3 (V3) were involved. The identical targeting vector 1 was used as described in FIG. 5. The targeting vector 3 contains the homologous arm sequences upstream and downstream of the mouse CD3E gene, and a fragment A3 containing the DNA sequences of human CD3E gene. Specifically in fragment A3, sequences of the upstream homologous arm (5′ homologous arm 2) and the downstream homologous arm (3′ homologous arm 2) in the targeting vector 3 are identical to sequences of the upstream and downstream homologous arms in the targeting vector 2 as described in FIG. 5. The fragment A3 also contains a human CD3E genomic DNA sequence (SEQ ID NO: 63), which is identical to nucleotide sequence of 118304953-118313732 of NCBI accession number NC_000011.10. The connection between the 5′ end of the human CD3E DNA sequence and the mouse sequence is identical to SEQ ID NO: 36. The connection between the 3′ end of the human CD3E DNA sequence and the mouse sequence was designed as:

(SEQ ID NO: 64)
5′-CCACAGTGTGTGAGAACTGCATGGAGATGGATCTGACAGCAGTAGC
CATAATCATCATTGTTGACATCT-3′,

wherein the last “T” in sequence “TGGAT” is the last nucleotide of the human sequence, and the first “C” in sequence “CTGAC” is the first nucleotide of the mouse sequence. The mRNA sequence of the engineered mouse CD3E after humanization and its encoded protein sequence are shown in SEQ ID NO: 65 and SEQ ID NO: 66, respectively.

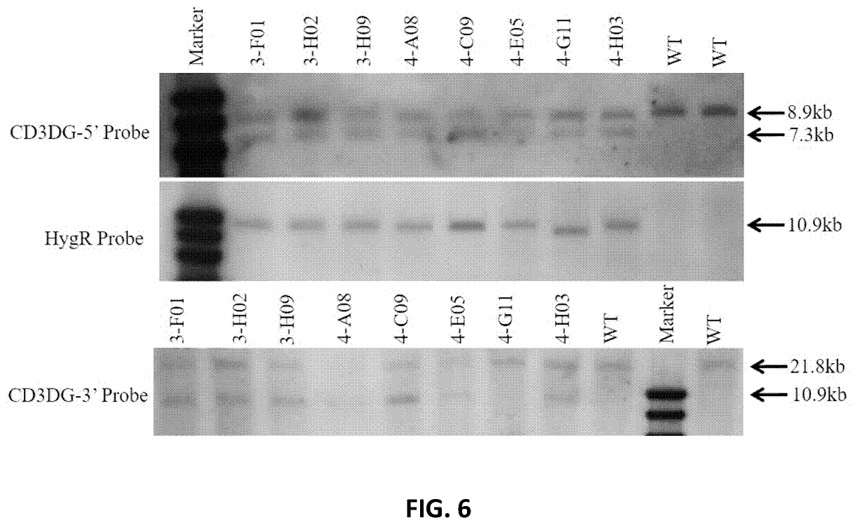

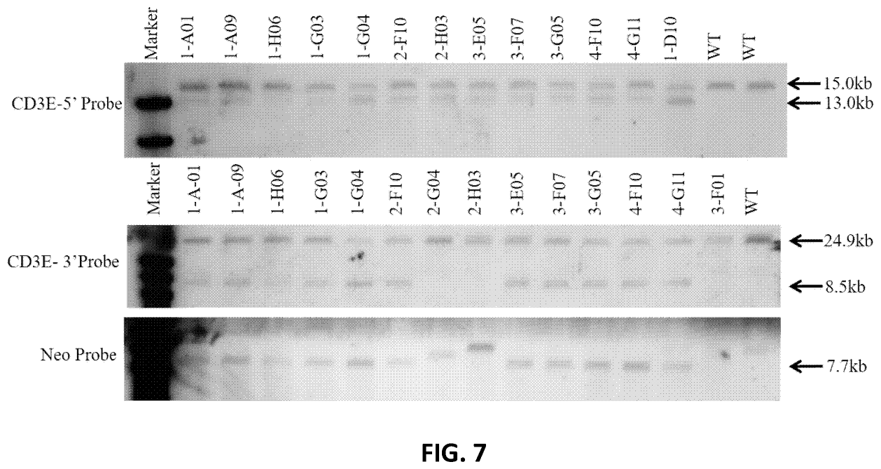

The targeting vectors were constructed, e.g., by restriction enzyme digestion and ligation. The constructed targeting vector sequences were preliminarily confirmed by restriction enzyme digestion, and then verified by sequencing. Embryonic stem cells of C57BL/6 mice or BALB/c mice were transfected with the correct targeting vectors by electroporating. The positive selectable marker genes were used to screen the cells, and the integration of exogenous genes was confirmed by PCR and Southern Blot after each step of the two-step targeting strategies. Specifically, after mouse embryonic stem cells were transfected with targeting vector 1, the clones identified as positive by CD3DG PCR primers were then verified by Southern Blot (cell DNA was digested with XbaI or SpeI, respectively, and hybridized with three probes) to screen out correct positive clone cells. The length of the probes and the size of target fragments are shown in the table below. As shown in FIG. 6, except for 4-G11, the other 7 clones numbered 3-F01, 3-H02, 3-H09, 4-A08, 4-009, 4-E05, and 4-H03 were identified as positive clones. The positive clones were further verified by sequencing, and no random insertions were detected. In the second step, positive clones with correct recombination of CD3D and CD3G genes (renumbered) were targeted by transfecting the cells with targeting vector 2 to humanize the mouse CD3E gene. As shown in FIG. 7, the clones identified as positive by CD3E PCR primers were then verified by Southern Blot (cell DNA was digested with NdeI, EcoRI, or SspI, respectively, and hybridized with three probes) to screen out correct positive clone cells. The length of the probes and the size of target fragments are shown in the table below. After sequencing, 6 clones numbered 1-A09, 2-F10, 3-E05, 3-G05, 4-F10, and 4-G11 were identified as positive clones.

TABLE 7
Specific probe and target fragment length

| Restriction | | | | | |
|---|---|---|---|---|---|
| enzyme | Probe | Wild-type | | Recombinant fragment size | |
| XbaI | CD3DG-5′Probe | 8.9 | kb | 7.3 | kb |
| SpeI | CD3DG-3′Probe | 21.8 | kb | 10.9 | kb |
| SpeI | HygR Probe | — | | 10.9 | kb |
| NdeI | CD3E-5′Probe | 15.0 | kb | 13.0 | kb |
| EcoRI | CD3E-3′Probe | 24.9 | kb | 8.5 | kb |

| | | | | |
|---|---|---|---|---|
| SspI | NeoProbe | — | 7.7 | kb |

The following primers were used in PCR:

CD3DG-F1:
(SEQ ID NO: 19)
5′-ACATTGTGCAACATGGTTCCTTTATGA-3′,
CD3DG-R1:
(SEQ ID NO: 20)
5′-GGCACCATTTTAGCTTCCTTTCGCC-3′;
CD3DG-F2:
(SEQ ID NO: 21)
5′-CTCGACTGTGCCTTCTAGTTGCCAG-3′,
CD3DG-R2:
(SEQ ID NO: 22)
5′-ACCCAAATTCCTTGTTTTCAGTTGCCA-3′;
CD3E-F1:
(SEQ ID NO: 23)
5′-GAGCTACACAGTGAGACTACCACAG-3′,
CD3E-R1:
(SEQ ID NO: 24)
5′-CACAGTATCTGACAATAAATGGCAAC-3′;
CD3E-F2:
(SEQ ID NO: 25)
5′-GCTCGACTAGAGCTTGCGGA-3′,
CD3E-R2:
(SEQ ID NO: 26)
5′-CTCTCAGTAATCAACTTCTCGTGTGTC-3′

The following primers were used for probe synthesis in Southern Blot assays:

CD3DG-5′Probe:
CD3DG-5′Probe-F:
(SEQ ID NO: 27)
5′-TACAGAACACATGGAGGCACAG-3′,
CD3DG-5′Probe-R:
(SEQ ID NO: 28)
5′-GTAACAACGCTGCAACAGGATC-3′;
CD3DG-3′Probe:
CD3DG-3′Probe-F:
(SEQ ID NO: 29)
5′-CACACTCTCATATCACTGTACACAC-3′,
CD3DG-3′Probe-R:
(SEQ ID NO: 30)
5′-TGATGGATCCAAGAGTGGGCAAC-3′;
HygR Probe:
HygR Probe-F:
(SEQ ID NO: 31)
5′-TCGATGTAGGAGGGCGTGGATATGT-3′
HygR Probe-R:
(SEQ ID NO: 32)
5′-TGTATTGACCGATTCCTTGCGGTCC-3′;
CD3E-5′Probe:
CD3E-5′Probe-F:
(SEQ ID NO: 33)
5′-GCTTCAAGGATGTTTAACTGCAAAG-3′,
CD3E-5′Probe-R:
(SEQ ID NO: 34)
5′-TGCAACAGATGTGCTGGCGA-3′;
CD3E-3′Probe:
CD3E-3′Probe-F:,
SEQ ID NO: 27
CD3E-3′Probe-R:;
SEQ ID NO: 28
NeoProbe:
NeoProbe-F:
(SEQ ID NO: 37)
5′-GGATCGGCCATTGAACAAGAT-3′,
NeoProbe-R:
(SEQ ID NO: 38)
5′-CAGAAGAACTCGTCAAGAAGGC-3′.

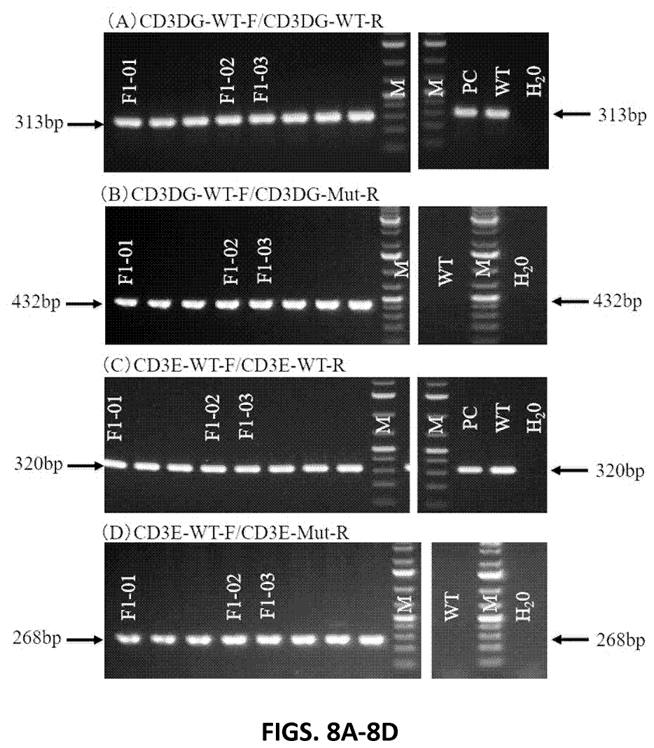

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the F1p transgenic mice (or Cre transgenic mice) to remove the positive selectable marker genes, and then the humanized homozygous mice with humanized CD3E, CD3D, and CD3G genes (hereinafter as humanized CD3EDG mice) were obtained by breeding the heterozygous mice with each other. The genotype of the progeny mice can be identified by PCR using primers shown in the table below. The identification results of exemplary F1 generation mice are shown in FIGS. 8A-8D. Mice numbered F1-01, F1-02, and F1-03 were identified as positive heterozygous clones.

TABLE 8
F1 generation genotype identification primer sequences and target fragment length

| Primer | SEQ ID NO | Sequence (5′-3′) | Fragment size |
|---|---|---|---|
| CD3DG-WT-F | SEQ ID NO: 39 | GATCCACACCATTGCACAAGCCAG | WT: 313 bp |
| CD3DG-WT-R | SEQ ID NO: 40 | TGCTTTGAGTTGTTGGTTGAGTTTC | |
| CD3DG-WT-F | SEQ ID NO: 39 | GATCCACACCATTGCACAAGCCAG | Mut: 432bp |
| CD3DG-Mut-R | SEQ ID NO: 41 | AAATGGGGATCAGAAACCCTTCACC | |
| CD3E-WT-F | SEQ ID NO: 42 | ATGGCAACCAATGATCCAGGGT | WT: 320 bp |
| CD3E-WT-R | SEQ ID NO: 43 | CTGAGTCCCCAGCCCTTGTC | |
| CD3E-WT-F | SEQ ID NO: 42 | ATGGCAACCAATGATCCAGGGT | Mut: 268 bp |
| CD3E-Mut-R | SEQ ID NO: 44 | ATGAGGCTCCTTGGTGCCACT | |
| Frt-F | SEQ ID NO: 45 | GCAGTGCTTCCTGTTAGAGGAGGTG | WT: 252 bp |
| Frt-R | SEQ ID NO: 46 | TTCTCGTCTTCCCTGGCTCTGTTCA | Mut: 339 bp |
| | | | (Neo cassette removed) |
| Lox-F | SEQ ID NO: 47 | TGTTGGGCACTTGTATTTGCATTTGT | Mut: 232 bp |
| Lox-R | SEQ ID NO: 48 | GGGATTGGGCTCAGGTTGTCACATT | (HygR cassette removed) |

The above results indicate that humanized CD3EDG mice can be generated using the method described herein. The mice can be stably passaged without random insertions.

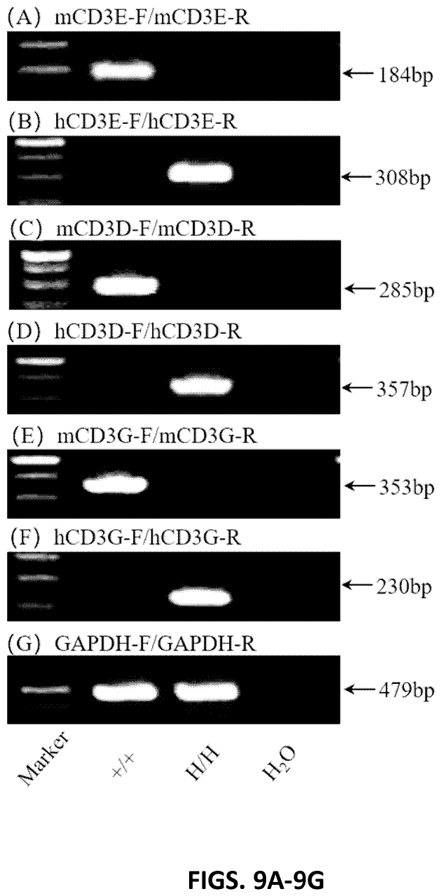

The expression of human CD3E, CD3D and CD3G mRNA in positive mice can be confirmed by RT-PCR. Specifically, one 8-week-old male C57BL/6 wild-type mouse and one 8-week-old male humanized CD3EDG homozygous mouse were selected. Thymus tissues were collected for RT-PCR detection after euthanasia. The primer sequences and fragment sizes used are shown in the table below. RT-PCR detection results are shown in FIGS. 9A-9G. Only mouse CD3E mRNA, CD3D mRNA and CD3G mRNA were detected in the thymus of C57BL/6 wild-type mouse (FIGS. 9A, 9C, and 9E). In contrast, only humanized CD3E mRNA, CD3D mRNA and CD3G mRNA were detected in the thymus of humanized CD3EDG homozygous mouse (FIGS. 9B, 9D, and 9F).

TABLE 9
RT-PCR primers and specific sequences

| Primer | SEQ ID NO | Sequence (5′-3′) | Fragment size |
|---|---|---|---|
| mCD3E-F | SEQ ID NO: 49 | GACGTGCCCTCTAGACAGTGACGAG | WT: 184 bp |
| mCD3E-R | SEQ ID NO: 50 | ACACACTCGAGCTTTCAGGTACAAG | |
| hCD3E-F | SEQ ID NO: 51 | TAGGCAGTGATGAGGATCACCTGTC | Mut: 308 bp |
| hCD3E-R | SEQ ID NO: 52 | CTTGTTTTGTCCCCTTTGCCTGCCG | |
| mCD3D-F | SEQ ID NO: 53 | AACGGTGGAAGGATGGTTTGCAAAG | WT: 285 bp |
| mCD3D-R | SEQ ID NO: 54 | GCTTGAACCTCAGCAGCCCCAGAAG | |
| hCD3D-F | SEQ ID NO: 55 | AATTGCAATACCAGCATCACATGGG | Mut: 357 bp |
| hCD3D-R | SEQ ID NO: 56 | ATCTCGGAGGGGCTGATAGACCTGG | |
| mCD3G-F | SEQ ID NO: 57 | AGCCCAGACAAATAAAGCAAAGAAT | WT: 353 bp |
| mCD3G-R | SEQ ID NO: 58 | GTCCCGCAATGAGATATACACCAAG | |
| hCD3G-F | SEQ ID NO: 59 | GGCCCAGTCAATCAAAGGAAACCAC | Mut: 230 bp |
| hCD3G-R | SEQ ID NO: 60 | GGAGTGGTTTTGACTTGTTCTGTGA | |
| GAPDH-F | SEQ ID NO: 61 | TCACCATCTTCCAGGAGCGAGA | WT: 479 bp |
| GAPDH-R | SEQ ID NO: 62 | GAAGGCCATGCCAGTGAGCTT | |

### Example 2. Phenotypic Identification of Humanized CD3EDG Mice

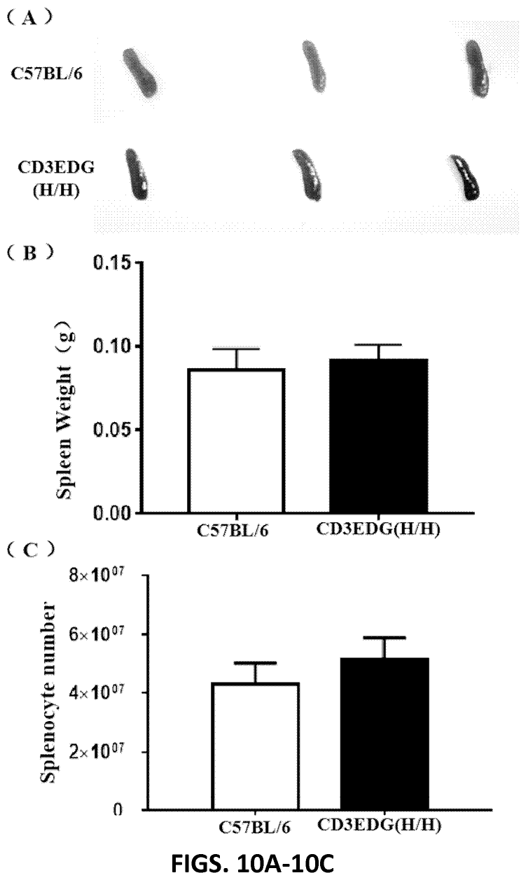

Three 7-week-old C57BL/6 wild-type mice and three humanized CD3EDG homozygous mice prepared in Example 1 were selected. The spleen and thymus tissues were isolated for comparison after euthanasia. FIGS. 10A-10C show the comparison results of spleen size, spleen weight and splenocyte number between C57BL/6 wild-type mice and humanized CD3EDG homozygous mice. As shown in FIG. 10A, the spleen of the humanized CD3EDG homozygous mice (H/H) and the spleen of the C57BL/6 wild-type mice had a similar size with an oblong shape. As shown in FIG. 10B, the spleen weight of humanized CD3EDG homozygous mice was comparable with the spleen weight of C57BL/6 wild-type mice. In addition, the number of splenocytes of humanized CD3EDG homozygous mice was similar to that of the C57BL/6 wild-type mice, with no significant difference (FIG. 10C).

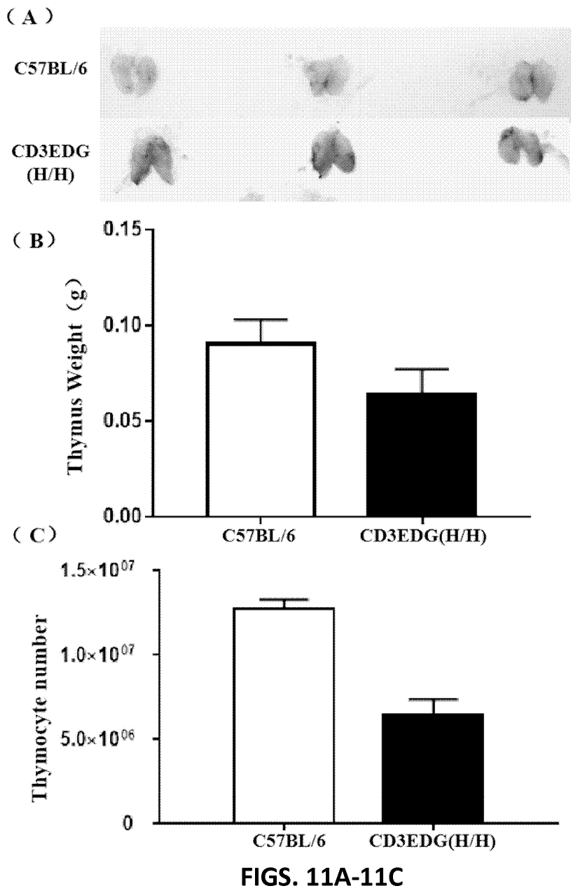

FIGS. 11A-11C show the comparison results of thymus size, thymus weight and thymocyte number between C57BL/6 wild-type mice and humanized CD3EDG homozygous mice. As shown in FIG. 11A, the thymus of the humanized CD3EDG homozygous mice (H/H) and the spleen of the C57BL/6 wild-type mice had a similar size. As shown in FIG. 11B, the thymus weight of humanized CD3EDG homozygous mice was smaller than that of C57BL/6 wild-type mice, but there was no statistically significant difference. The number of thymocytes in humanized CD3EDG homozygous mice was smaller than that of C57BL/6 wild-type mice, but there was no significant difference (FIG. 11C).

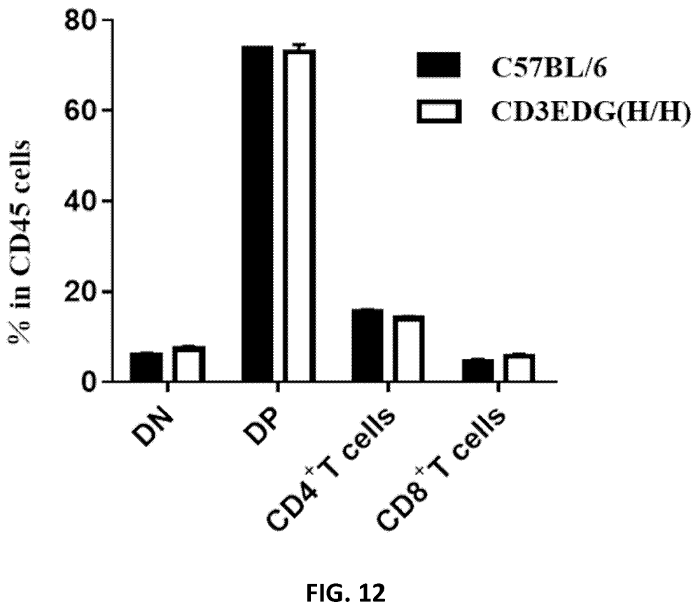

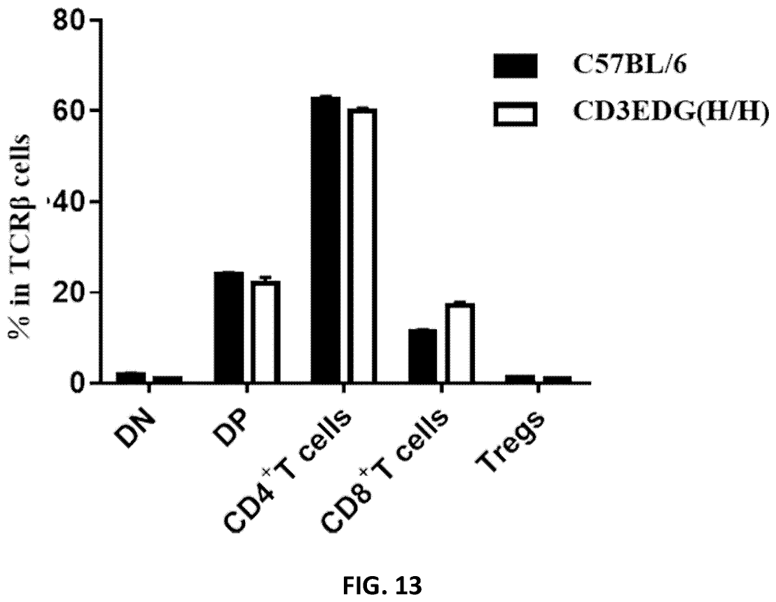

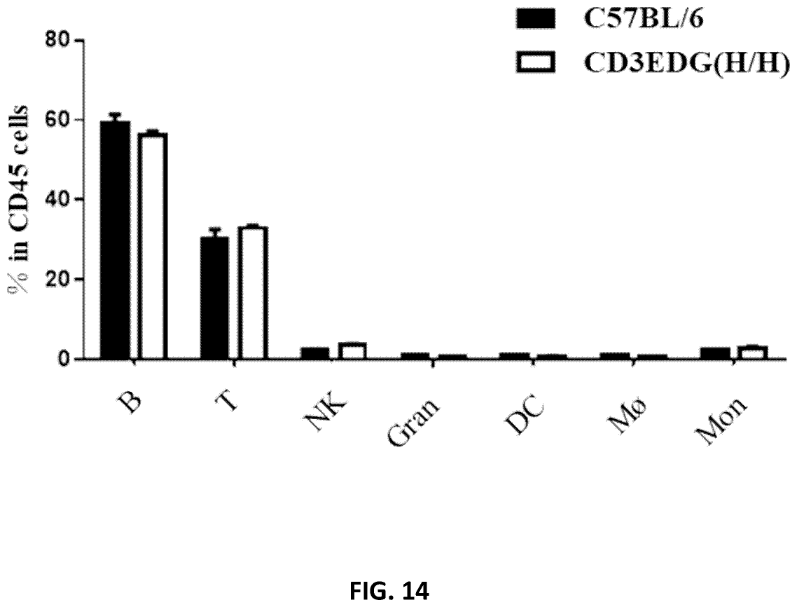

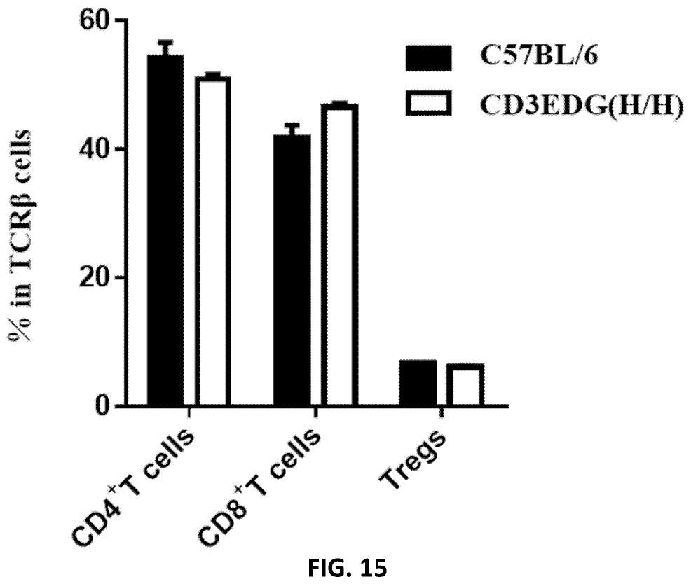

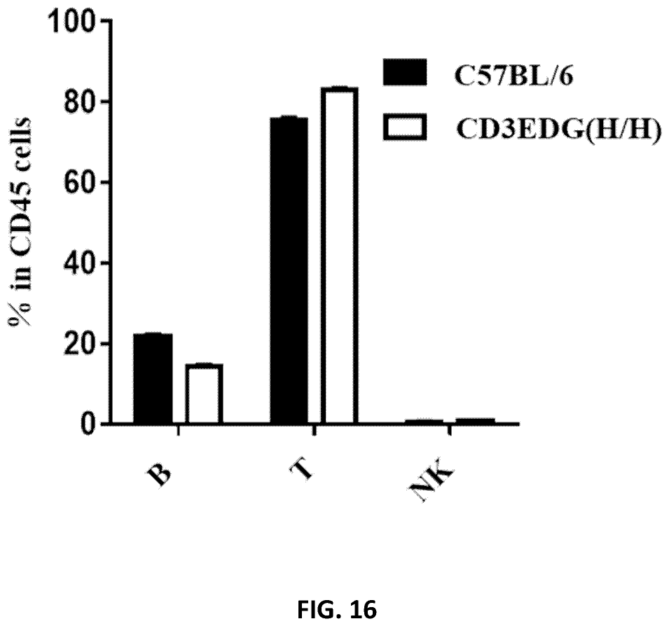

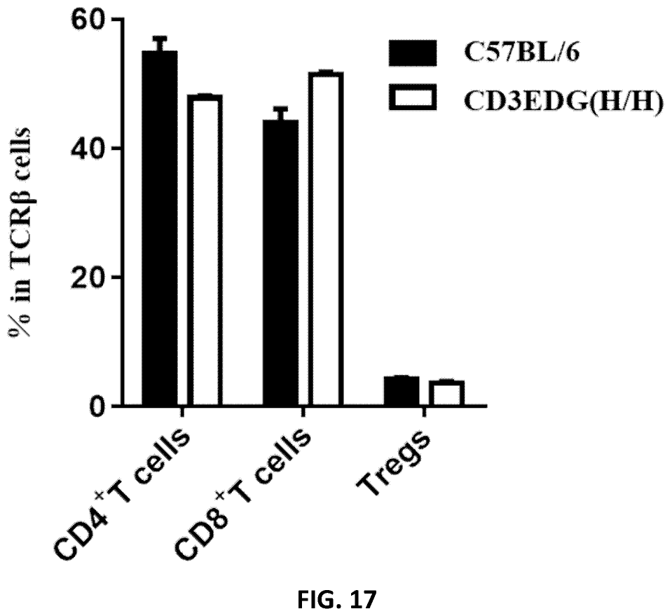

Leukocyte subtypes and T cell subtypes in lymphoid tissues (e.g., thymus, spleen and lymph nodes) of 7-week-old female C57BL/6 wild-type mice and humanized CD3EDG homozygous mice (H/H) were further detected by flow cytometry. The detection results of leukocyte subtypes and T cell subtypes in the thymus are shown in FIG. 12 and FIG. 13, respectively. The detection results of leukocyte subtypes and T cell subtypes in the spleen are shown in FIG. 14 and FIG. 15, respectively. The detection results of leukocyte subtypes and T cell subtype in lymph nodes are shown in FIG. 16 and FIG. 17, respectively. The results show that the percentages of T cells (“T” in FIG. 14 and FIG. 16), B cells (“B” in FIG. 14 and FIG. 16), NK cells (“NK” in FIG. 14 and FIG. 16), DC cells (“DC” in FIG. 14), granulocytes (“Gran” in FIG. 14), monocytes (“Mon” in FIG. 14), macrophages (“Mo” in FIG. 14), and other leukocytes subtypes in the humanized CD3EDG homozygous mice were comparable to those in C57BL/6 wild-type mice. In addition, the percentages of T cells such as CD4+ T cells (in FIG. 12, FIG. 13, FIG. 15, and FIG. 17), CD8+ T cells (in FIG. 12, FIG. 13, FIG. 15, and FIG. 17) and Tregs (in FIG. 13, FIG. 15, and FIG. 17) and other T cell subtypes in the humanized CD3EDG homozygous mice were comparable to those in C57BL/6 wild-type mice. The results indicate that humanization of CD3E, CD3D and CD3G genes did not affect the differentiation, development and distribution of leukocytes in lymphoid tissues.

### Example 3. T Cell Activation Assays in Humanized CD3EDG Mice

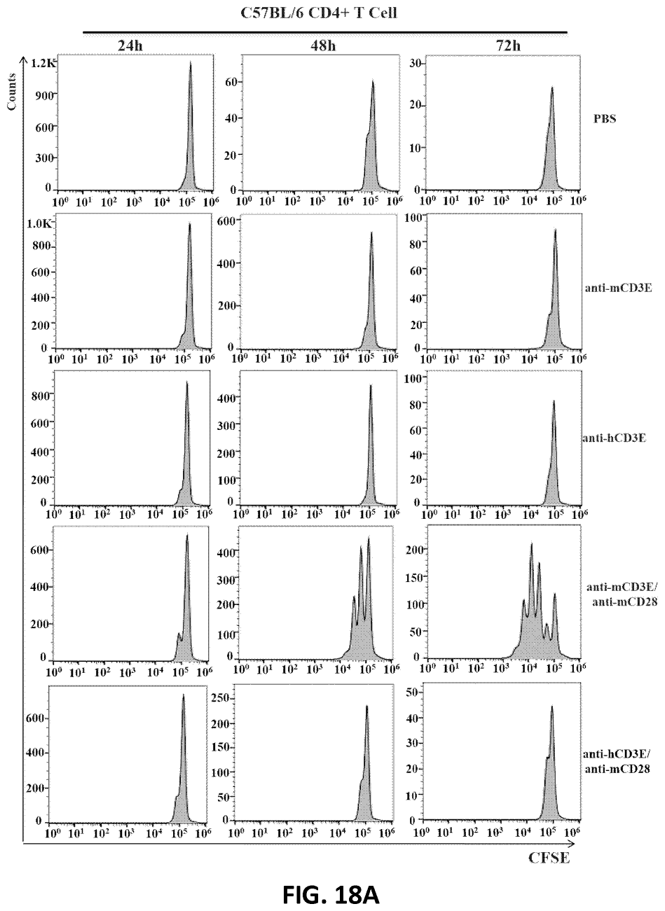

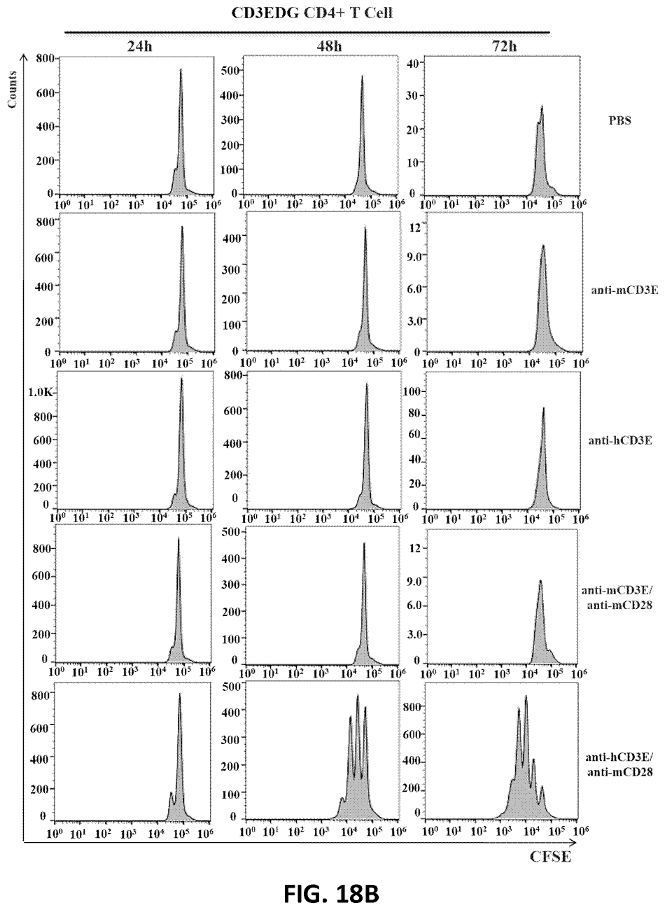

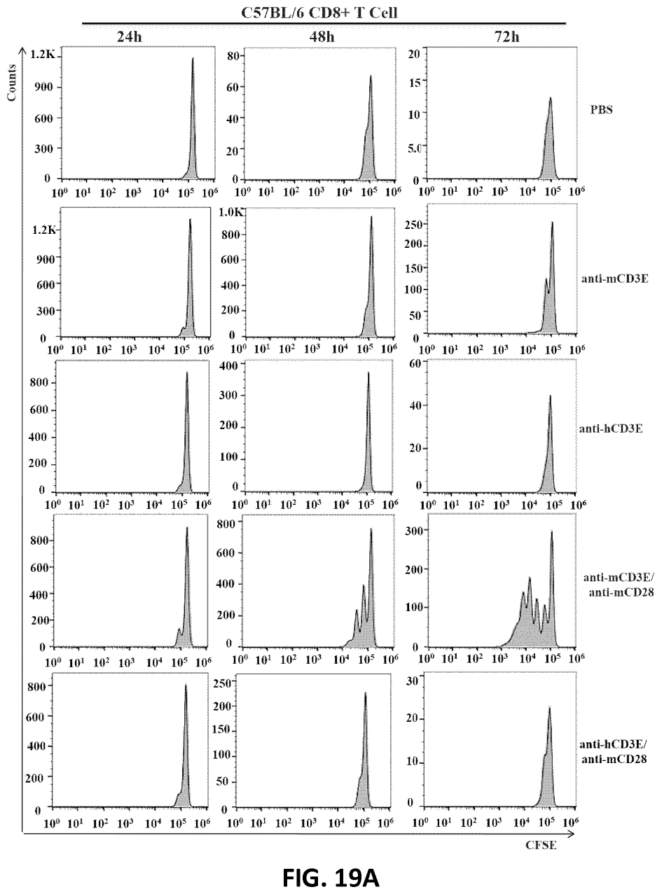

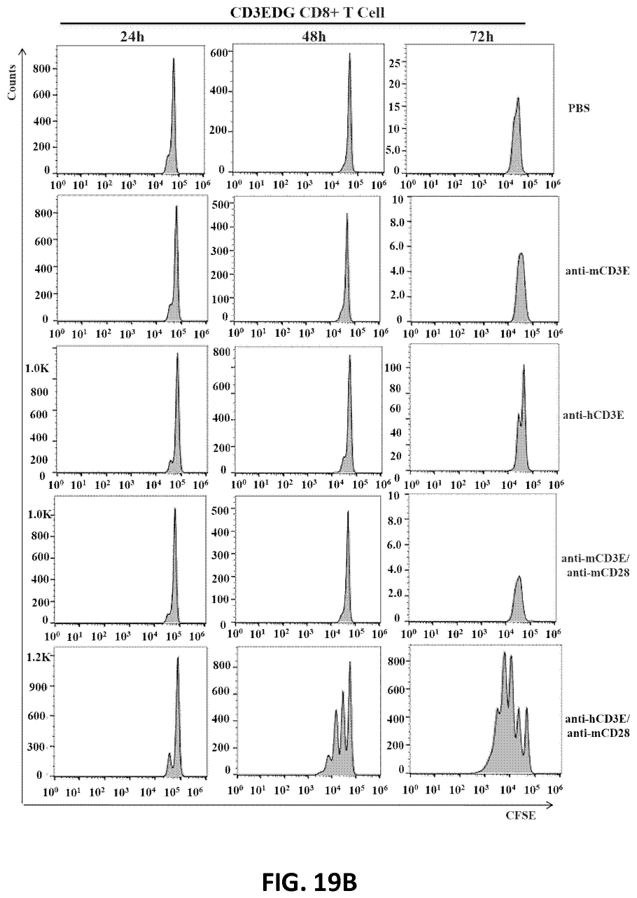

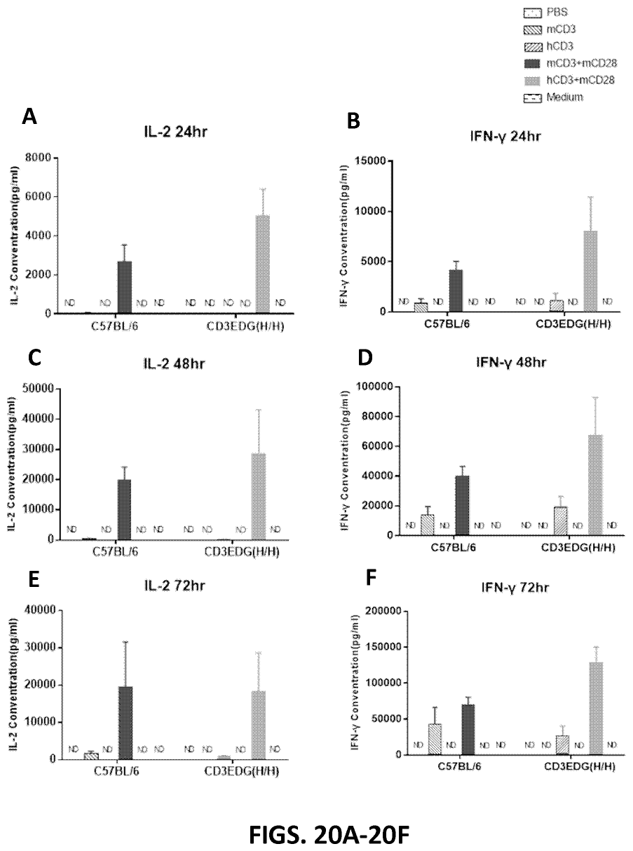

Three C57BL/6 wild-type mice and three humanized CD3EDG homozygous mice (16 weeks old) prepared in Example 1 were selected. The spleen tissues were isolated after euthanasia. T cells (2×10⁵ cells) were collected and incubated with 2 μg/ml of an anti-mouse CD3E antibody (anti-mCD3E), anti-human CD3E antibody (anti-hCD3E), or in combination with 5 μg/ml of an anti-mouse CD28 antibody (anti-mCD28). The proliferation of CD4+ T cells and CD8+ T cells was detected by flow cytometry at 24 hours, 48 hours, and 72 hours, respectively. The flow cytometry results are shown in FIGS. 18A-18B and FIGS. 19A-19B, respectively. ELISA was also used to detect the expression of IFN-γ and IL-2 in the spleen tissues of C57BL/6 wild-type mice and humanized CD3EDG homozygous mice at 24 hours, 48 hours, and 72 hours, respectively, to further verify the activation status of CD4+ T cells and CD8+ T cells. Specific test results are shown in FIGS. 20A-20F.

FIGS. 18A-18B show the detection results of spleen CD4+ T cells of C57BL/6 wild-type mice and humanized CD3EDG homozygous mice, respectively. As shown in FIG. 18A, for C57BL/6 wild-type mice, after treatment of PBS, anti-mCD3E, anti-hCD3E, or a combination of the anti-human CD3E antibody and the anti-mouse CD28 antibody (anti-hCD3E/anti-mCD28), the fluorescence intensity of CD4+ T cells was consistent. No obvious proliferation change was observed, and the results showed a single peak. However, after treatment with the anti-mCD3E antibody and anti-mCD28 antibody (anti-mCD3E/anti-mCD28), CD4+ T cells proliferated significantly, showing a series of sub-peaks with half-decay in CSFE fluorescence intensity. According to the number of sub-peaks, it is contemplated that the cell group underwent approximately 5 divisions at 72 hours. As shown in FIG. 18B, for humanized CD3EDG mice, CD4+ T cells did not undergo significant proliferation changes after treatment with anti-mCD3E/anti-mCD28, and the results showed a single peak. However, after treatment with the anti-hCD3E antibody and anti-mCD28 antibody (anti-hCD3E/anti-mCD28), CD4+ T cells proliferated significantly.

FIGS. 19A-19B show the detection results of spleen CD8+ T cells of C57BL/6 wild-type mice and humanized CD3EDG homozygous mice, respectively. As shown in FIG. 19A, for C57BL/6 wild-type mice, after treatment of PBS, anti-mCD3E, anti-hCD3E, or anti-hCD3E/anti-mCD28, the fluorescence intensity of CD8+ T cells was consistent. No obvious proliferation change was observed, and the results showed a single peak. However, after treatment with anti-mCD3E/anti-mCD28, CD8+ T cells proliferated significantly, showing a series of sub-peaks with half-decay in CSFE fluorescence intensity. According to the number of sub-peaks, it is contemplated that the cell group underwent approximately 5 divisions at 72 hours. As shown in FIG. 19B, for humanized CD3EDG mice, CD8+ T cells did not undergo significant proliferation changes after treatment with anti-mCD3E/anti-mCD28, and the results showed a single peak. However, after treatment with anti-hCD3E/anti-mCD28, CD8+ T cells proliferated significantly.

As shown in FIGS. 20A-20F, only when the C57BL/6 wild-type mice were treated with both an anti-mouse CD3E antibody (anti-mCD3E) and an anti-mouse CD28 antibody (anti-mCD28); or when the humanized CD3EDG homozygous mice were treated with an anti-human CD3E antibody (anti-hCD3E) and anti-mCD28, both IL-2 and IFN-γ could be effectively activated at 24 hours, 48 hours, and 72 hours. No significant difference of expression was observed.

### Example 4. Detection of OVA-Specific Antibody Titers in Humanized CD3EDG Mouse Serum

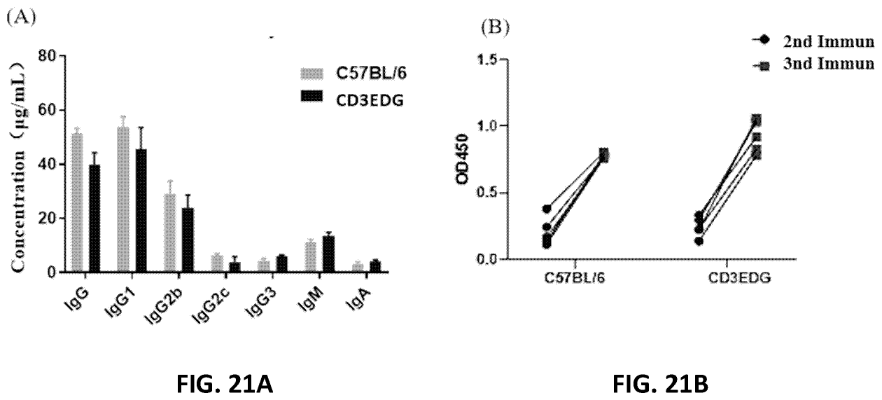

Five 6-week-old C57BL/6 wild-type mice and five humanized CD3EDG mice prepared in Example 1 were selected. Orbital blood was collected (50 μl), and serum was isolated. Mouse total IgG as well as IgA, IgM, IgG1, IgG2b, IgG2c, and IgG3 subtypes were quantitatively detected using Goat Anti-Mouse Ig, Human ads-UNLB; Goat Anti-Mouse IgA-HRP; Goat Anti-Mouse IgM, Human ads-HRP; Goat Anti-Mouse IgG1, Human ads-HRP; Goat Anti-Mouse IgG2b, Human ads-HRP; Goat Anti-Mouse IgG2c, Human ads-HRP; and Goat Anti-Mouse IgG3, Human ads-HRP, respectively, from the SBA Clonotyping System-057BL/6-HRP kit. Afterwards, 50 μg of OVA (ovalbumin) was administered subcutaneously in four locations on the back for immunization. The first subcutaneous injection was emulsified with Complete Freund's Adjuvant (CFA), and subsequent subcutaneous injections were emulsified with Incomplete Freund's Adjuvant (IFA). Blood (serum) was collected one week after the third injection and analyzed for antibody titers using ELISA with a 14-day interval between immunizations. The results are shown in FIGS. 21A-21B.

As shown in FIG. 21A, before immunization, there was no significant difference of the serum levels of antibody subtypes in humanized CD3EDG mice as compared to C57BL/6 wild-type mice. As shown in FIG. 21B, after the third immunization, the titers of OVA-specific antibodies in the serum of both C57BL/6 wild-type mice and humanized CD3EDG mice significantly increased. The results indicate that humanization of CD3E, CD3D and CD3G genes did not affect the humoral immune response in humanized CD3EDG mice.

### Example 5. Pharmacological Validation of Humanized CD3EDG Mice (1)

Fifteen 8-week-old female humanized CD3EDG homozygous mice as prepared in Example 1 were subcutaneously injected with 5×10⁵ mouse colon cancer cell line MC38. When the tumor volume grew to about 100±50 mm³, the mice were randomly placed into a control group (G1) and two treatment groups (G2 and G3) based on tumor size (5 mice per group). The G1 group mice were administered with an anti-human IgG antibody (hIgG Ab) at 2 mg/kg; the G2 group mice were administered with an anti-mouse PD-1 antibody (mPD-1 Ab; obtained by immunizing mice using methods described in Janeway's Immunobiology (9th Edition)) at 10 mg/kg; the G3 group mice were administered with an anti-human CD3E antibody (hCD3E Ab, obtained from Provention Bio, Inc.) at 2 mg/kg. All antibodies were administered via intraperitoneal injection (i.p.). The mice were administered on the grouping day, and the frequency of administration was twice a week (6 times of administrations in total). The tumor volume was measured twice a week and the body weight of the mice was weighed as well. Euthanasia was performed when the tumor volume of the mouse reached 3000 mm³.

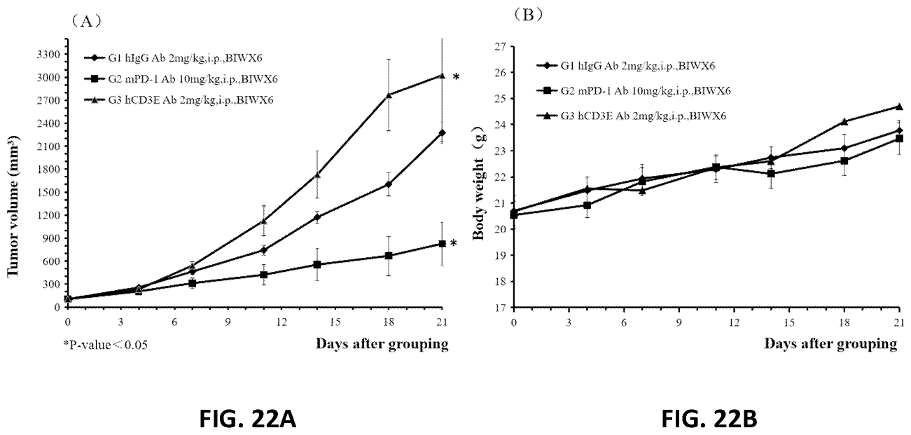

The tumor volume change in mice and the change of mouse body weight are shown in FIGS. 22A-22B, respectively. Overall, the animals in each group were healthy, and the body weights of all the treatment group mice (G2, G3) and control group mice (G1) increased, and were not significantly different from each other during the experimental period (FIG. 22B). According to the results of tumor volume measurement (FIG. 22A), the tumor volume of each mouse in the control group (G1) and treatment groups (G2, G3) continued to grow during the experimental period. Compared with the control group, the tumor volume in the G2 group mice was significantly reduced, while the tumor growth rate in the G3 group mice was significantly higher than that of the control group mice. This may be due to the activation-induced cell death (AICD) effect caused by anti-human CD3E antibody treatment in the G3 group, which exhausted T cells and promoted the growth of tumor tissues. The results indicate that the in vivo function of T cells in humanized CD3EDG mice was normal.

Table 4 shows results for this experiment, including the tumor volumes on the day of grouping (Day 0), 14 days after the grouping (Day 14), and at the end of the experiment (Day 18), the survival rate of the mice, the Tumor Growth Inhibition value (TGI^{TV}%), and the statistical differences (P value) of mouse body weight and tumor volume between the treatment and control groups.

TABLE 10
Tumor volume, survival and Tumor Growth Inhibition value

| | | | | | | | P value | |
|---|---|---|---|---|---|---|---|---|
| | | Tumor volume (mm³) | | | | | Body | Tumor |
| | | Day 0 | Day 14 | Day 18 | Survival | TGI_{TV} % | weight | Volume |
| Control | G1: | 104 ± 4 | 1172 ± 79 | 1603 ± 151 | 5/5 | N/A | N/A | N/A |
| Treatment | G2: | 104 ± 4 | 554 ± 207 | 668 ± 256 | 5/5 | 62.3% | 0.547 | 0.014 |
| | G3: | 104 ± 5 | 1729 ± 310 | 2769 ± 465 | 5/5 | −77.8% | 0.318 | 0.044 |

As shown in the above table, all mice in the control group and the treatment groups survived at the end of the experiment, and there was no significant difference in animal body weight between all the treatment group mice and the control group mice (p>0.05). The results indicate that the anti-mouse PD-1 antibody mPD-1 Ab and the anti-human CD3 antibody hCD3E Ab were well tolerated and not toxic to the mice. Tumors continued to grow in all mice in the control group (G1) during the experiment. At the end of the experiment, the average tumor volume was 1603±151 mm³ in the G1 group, 668±256 mm³ in the G2 group, and 2769±465 mm 3 in the G3 group. The tumor volume of mice in all treatment groups was significantly different from that of the control group (P≤0.05). The TGI_{TV}% value for the G2 and G3 group mice were 62.3% and −77.8%, respectively. However, the tumor volume of the G3 group mice was significantly higher than that of the control group mice, which may be due to the AICD effect caused by the anti-human CD3E antibody treatment in the G3 group, which exhausted T cells and promoted tumor tissue growth.

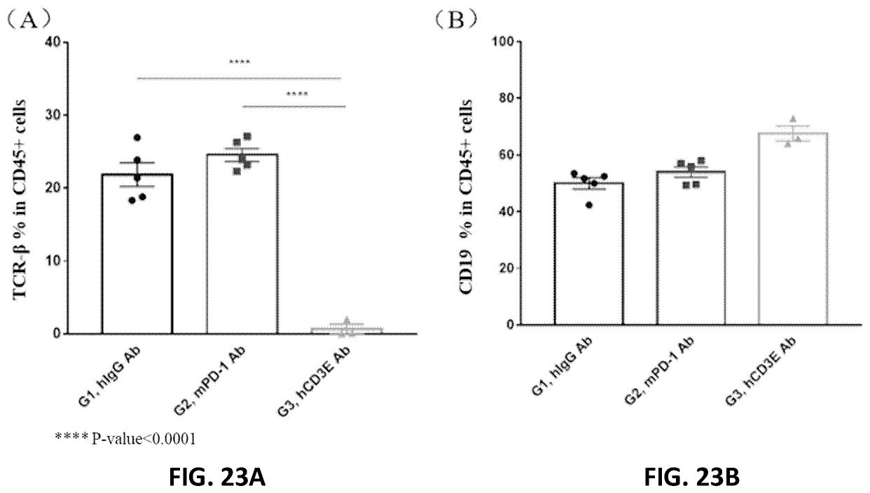

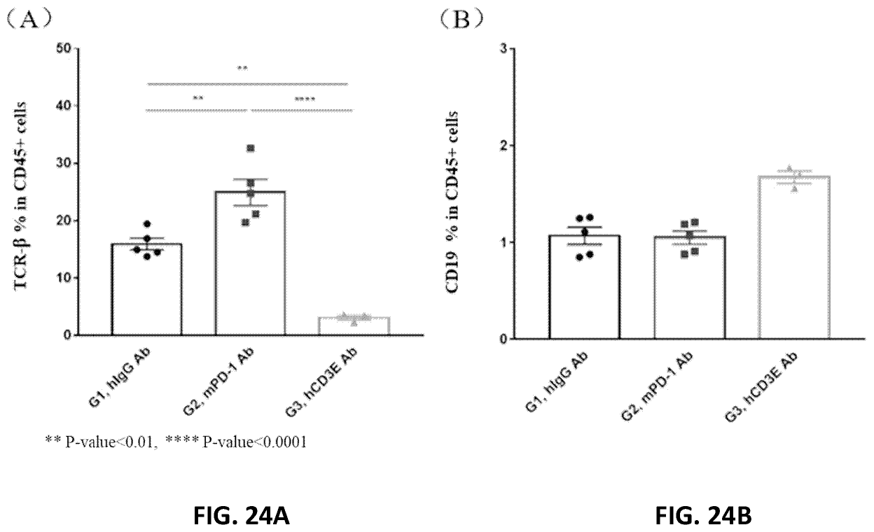

The ratio of B cells and T cells in peripheral blood and tumor tissue of mice after administration was further detected by flow cytometry. After 48 hours of administration, lymphocytes were separated from peripheral blood and tumor tissue of each mouse, and the changes of T cells and B cells were detected. FIGS. 23A-23B show the detection results in peripheral blood, and FIGS. 24A-24B show the detection results in tumor tissue.

As shown in FIG. 23A, compared with the control group mice (G1), the percentage of T cells in the peripheral blood of the G3 group mice was significantly reduced, which may be due to the AICD effect caused by anti-human CD3E antibody treatment. The percentage of T cells in the anti-mouse PD-1 antibody treatment group mice (G2) increased slightly, but the difference did not reach statistical significance. As shown in FIG. 23B, compared with the control group mice (G1), the percentage of B cells in the peripheral blood of the anti-mouse PD-L1 antibody treatment group mice (G2) and the anti-human CD3E antibody treatment group mice (G3) slightly increased, but there was no statistical difference.

As shown in FIG. 24A, compared with the control group mice (G1), the percentage of T cells in the tumor tissue of the G3 group mice was significantly reduced. The percentage of T cells in the anti-mouse PD-1 antibody treatment group mice (G2) significantly increased because the PD-1 antibody relieved the immunosuppressive effect. As shown in FIG. 24B, compared with the control group mice (G1), there was no significant change in the percentage of B cells in the anti-mouse PD-L1 antibody treatment group mice (G2) and the anti-human CD3E antibody treatment group mice (G3).

The above experiments demonstrate that the humanized CD3EDG mice can be used as a favorable tool for the in vivo pharmacological and pharmacodynamic study of anti-CD3 antibody.

### Example 6. In Vitro Killing Assay of CD3EDG Humanized Mice

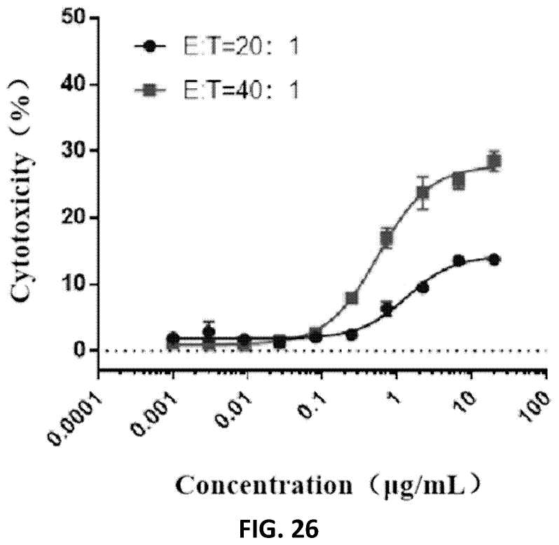

MC38 cells with high expression of human BCMA protein were seeded into a 96-well plate as target cells. The 6-8 week-old female humanized CD3EDG mice prepared in Example 1 were euthanized to obtain splenocytes as effector cells. The experimental group was added with an anti-human CD3/BCMA bispecific antibody Abl at concentrations of 20.000, 6.667, 2.222, 0.741, 0.247, 0.082, 0.027, 0.009, 0.003, and 0.001 μg/mL, respectively. The control group was added with an equal amount of the dilution solution of Abl. The plate was incubated in a 37° C., 5% CO 2 incubator for 48 hours, and supernatant was collected for LDH (lactate dehydrogenase) detection. FIG. 26 shows the detection results when the effector to target cell count ratio (E:T) was 20:1 and 40:1, respectively. The results showed that after the 48-hour incubation, the killing effect of Abl on target cells was detected in both E:T=20:1 and E:T=40:1 groups. In particular, the killing effect was more obviously observed in the E:T=40:1 group.

### Example 7. Pharmacological Validation of Humanized CD3EDG Mice (2)

Twenty 6-week-old female humanized CD3EDG homozygous mice as prepared in Example 1 were subcutaneously injected with 5×10⁵ transfected MC38 cells with high expression of human BCMA protein. When the tumor volume grew to about 100±50 mm³, the mice were randomly placed into a control group (G1) and three treatment groups (G2, G3, and G4) based on tumor size (5 mice per group). The G1 group mice were administered with saline (0.1 mg/kg), the G2-G4 group mice were administered with an anti-human CD3/BCMA bispecific antibody (Abl; obtained by immunizing mice using methods described in Janeway's Immunobiology (9th Edition)). All antibodies were administered by tail vein injection. The mice were administered on the grouping day, and the frequency of administration was once every 5 days (4 times of administrations in total). The first three doses of G2, G3, and G4 groups were 0.001 mg/kg, 0.01 mg/kg, and 0.1 mg/kg, respectively, and the fourth dose was 0.01 mg/kg, 0.1 mg/kg, and 0.1 mg/kg, respectively. The tumor volume was measured twice a week and the body weight of the mice was weighed as well. Euthanasia was performed when the tumor volume of the mouse reached 3000 mm³.

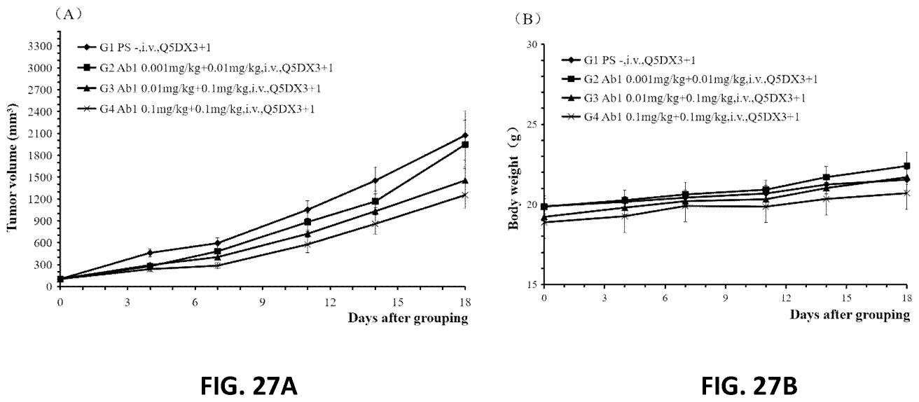

The tumor volume change in mice and the change of mouse body weight are shown in FIGS. 27A-27B, respectively. Overall, the animals in each group were healthy. At the end of the experiment, the body weight of animals in each group increased to a certain extent (FIG. 27B). According to the results of tumor volume measurement (FIG. 27A), the tumor volume of each mouse in the control group (GI) and treatment groups (G2, G3, and G4) continued to grow during the experimental period. However, compared with the control group mice, the tumor volume growth of the treatment group mice slowed down. The results indicate that humanized CD3EDG mice can be used to evaluate the efficacy of antibody drugs targeting human CD3 protein, and it is a favorable tool for the study of the in vivo pharmacological efficacy of antibodies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric CD3D.

2. The animal of claim 1, wherein the sequence encodes a human CD3D.

3. The animal of claim 1 or 2, wherein the sequence encoding the human or chimeric CD3D is operably linked to a human regulatory element (e.g., 5′ UTR and/or 3′ UTR) at an endogenous CD3D gene locus in the at least one chromosome.

4. The animal of any one of claims 1-3, wherein the human or chimeric CD3D comprises an amino acid sequence that is at least 70%, 75%, 80%, 85%, 90%, 95%, 96%, 97%, 98%, 99%, or 100% identical to human CD3D (NP_000723.1 (SEQ ID NO: 4)).

5. The animal of any one of claims 1-4, wherein the animal is a mammal, e.g., a monkey, a rodent, or a mouse.

6. The animal of any one of claims 1-5, wherein the animal is a mouse.

7. The animal of any one of claims 1-6, wherein the animal does not express endogenous CD3D.

8. The animal of any one of claims 1-7, wherein the animal has one or more cells expressing human or chimeric CD3D.

9. The animal of claim 8, wherein the animal has one or more cells expressing human CD3D, wherein the expressed human CD3D can form a CD3 complex with human or humanized CD3E, human or humanized CD3G, and endogenous CD3-zeta polypeptides.

10. A genetically-modified, non-human animal, wherein the genome of the animal comprises a replacement of a sequence encoding a region of endogenous CD3D with a sequence encoding a corresponding region of human CD3D at an endogenous CD3D gene locus.

11. The animal of claim 10, wherein the sequence encoding the corresponding region of human CD3D is operably linked to a human regulatory element (e.g., 5′ UTR and/or 3′ UTR) at the endogenous CD3D locus, and one or more cells of the animal expresses human or chimeric CD3D.

12. The animal of claim 10 or 11, wherein the animal does not express endogenous CD3D.

13. The animal of any one of claims 10-12, wherein the sequence that is replaced encodes full-length endogenous CD3D.

14. The animal of any one of claims 10-12, wherein the animal is a mouse, and the sequence that is replaced comprises exon 1, exon 2, exon 3, exon 4, and/or exon 5 of the endogenous mouse CD3D gene.

15. The animal of claim 14, wherein the sequence that is replaced further comprises 5′ UTR and/or 3′ UTR of the endogenous mouse CD3D gene.

16. The animal of any one of claims 10-15, wherein the animal is heterozygous with respect to the replacement at the endogenous CD3D gene locus.

17. The animal of any one of claims 10-15, wherein the animal is homozygous with respect to the replacement at the endogenous CD3D gene locus.

18. A non-human animal comprising a nucleotide sequence encoding a chimeric CD3D polypeptide, wherein the chimeric CD3D polypeptide comprises a human CD3D signal peptide, and at least 50 contiguous amino acid residues that are identical to the corresponding contiguous amino acid sequence of a human CD3D, wherein the animal expresses the chimeric CD3D polypeptide.

19. The animal of claim 18, wherein the chimeric CD3D polypeptide has at least 50, at least 60, at least 70, at least 80, at least 90, at least 100, at least 110, at least 120, at least 130, at least 140, at least 150, at least 160, or at least 170 contiguous amino acid residues that are identical to the corresponding contiguous amino acid sequence of a human CD3D.

20. The animal of claim 18 or 19, wherein the chimeric CD3D polypeptide comprises a sequence that is at least 90%, 95%, 96%, 97%, 98%, 99%, or 100% identical to SEQ ID NO: 4.

21. The animal of any one of claims 18-20, wherein the nucleotide sequence is integrated to an endogenous CD3D gene locus of the animal.

22. The animal of anyone of claims 1-21, wherein the animal further comprises a sequence encoding an additional human or chimeric protein.

23. The animal of claim 22, wherein the additional human or chimeric protein is CD3E, CD3G programmed cell death protein 1 (PD-1), Programmed Cell Death 1 Ligand 1 (PD-L1), cytotoxic T-lymphocyte-associated protein 4 (CTLA-4), Lymphocyte Activating 3 (LAG-3), B And T Lymphocyte Associated (BTLA), tumor necrosis factor receptor superfamily member 9 (4-1BB), T-Cell Immunoreceptor With Ig And ITIM Domains (TIGIT), B7 Homolog 3 (B7-H3), T-cell Immunoglobulin and Mucin-Domain Containing-3 (TIM-3), Glucocorticoid-Induced TNFR-Related Protein (GITR), TNF Receptor Superfamily Member 4 (OX40), CD27, CD28, CD38, CD47, CD154, CD40, CD278, or CLDN18.2.

24. A method for making a genetically-modified, non-human animal, comprising:
replacing in at least one cell of the animal, at an endogenous CD3D gene locus, a sequence encoding a region of an endogenous CD3D with a sequence encoding a corresponding region of human CD3D.

25. The method of claim 24, wherein the sequence encoding the corresponding region of human CD3D comprises exon 1, exon 2, exon 3, exon 4, and/or exon 5, or a part thereof, of a human CD3D gene.

26. The method of claim 24 or 25, wherein the sequence encoding the corresponding region of human CD3D encodes SEQ ID NO: 4.

27. The method of any one of claims 24-26, wherein the animal is a mouse, and the endogenous CD3D locus comprises exon 1, exon 2, exon 3, exon 4, and/or exon 5, or a part thereof, of the mouse CD3D gene.

28. A method of making a genetically-modified non-human animal (e.g., mouse) cell that expresses a human or chimeric CD3D, the method comprising:
replacing at an endogenous CD3D gene locus, a nucleotide sequence encoding a region of endogenous CD3D with a nucleotide sequence encoding a corresponding region of human CD3D, thereby generating a genetically-modified non-human animal cell that includes a nucleotide sequence that encodes the human or chimeric CD3D, wherein the non-human animal cell expresses the human or chimeric CD3D.

29. The method of claim 28, wherein the nucleotide sequence encoding the human or chimeric CD3D is operably linked to a human CD3D regulatory region, e.g., promoter.

30. The method of claim 28, wherein the nucleotide sequence encoding the human or chimeric CD3D is operably linked to an endogenous CD3D regulatory region, e.g., promoter.

31. The method of anyone of claims 24-30, wherein the animal further comprises a sequence encoding an additional human or chimeric protein.

32. The method of claim 31, wherein the additional human or chimeric protein is CD3E, CD3G PD-1, PD-L1, CTLA-4, LAG-3, BTLA, 4-1BB, TIGIT, B7-H3, TIM-3, GITR, OX40, CD27, CD28, CD38, CD47, CD154, CD40, CD278, and/or CLDN18.2.

33. A genetically-modified, non-human animal whose genome comprises at least one chromosome comprising a sequence encoding a human or chimeric CD3G.

34. The animal of claim 33, wherein the sequence encodes a human CD3G.

35. The animal of claim 33 or 34, wherein the sequence encoding the human or chimeric CD3G is operably linked to a human regulatory element (e.g., 5′ UTR and/or 3′ UTR) at an endogenous CD3G gene locus in the at least one chromosome.

36. The animal of any one of claims 33-35, wherein the human or chimeric CD3G comprises an amino acid sequence that is at least 70%, 75%, 80%, 85%, 90%, 95%, 96%, 97%, 98%, 99%, or 100% identical to human CD3G (NP_000064.1 (SEQ ID NO: 6)).

37. The animal of any one of claims 33-36, wherein the animal is a mammal, e.g., a monkey, a rodent, or a mouse.

38. The animal of any one of claims 33-37, wherein the animal is a mouse.

39. The animal of any one of claims 33-38, wherein the animal does not express endogenous CD3G.

40. The animal of any one of claims 33-39, wherein the animal has one or more cells expressing human or chimeric CD3G.

41. The animal of claim 40, wherein the animal has one or more cells expressing human CD3G wherein the expressed human CD3G can form a CD3 complex with human or humanized CD3E, human or humanized CD3D, and endogenous CD3-zeta polypeptides.

42. A genetically-modified, non-human animal, wherein the genome of the animal comprises a replacement of a sequence encoding a region of endogenous CD3G with a sequence encoding a corresponding region of human CD3G at an endogenous CD3G gene locus.

43. The animal of claim 42, wherein the sequence encoding the corresponding region of human CD3G is operably linked to a human regulatory element (e.g., 5′ UTR and/or 3′ UTR) at the endogenous CD3G locus, and one or more cells of the animal expresses human or chimeric CD3G.

44. The animal of claim 42 or 43, wherein the animal does not express endogenous CD3G.

45. The animal of any one of claims 42-44, wherein the sequence that is replaced encodes full-length endogenous CD3G.

46. The animal of any one of claims 42-45, wherein the animal is a mouse, and the sequence that is replaced comprises exon 1, exon 2, exon 3, exon 4, exon 5, exon 6, and/or exon 7 of the endogenous mouse CD3G gene.

47. The animal of claim 46, wherein the sequence that is replaced further comprises 5′ UTR and/or 3′ UTR of the endogenous mouse CD3G gene.

48. The animal of any one of claims 42-47, wherein the animal is heterozygous with respect to the replacement at the endogenous CD3G gene locus.

49. The animal of any one of claims 42-47, wherein the animal is homozygous with respect to the replacement at the endogenous CD3G gene locus.

50. A non-human animal comprising a nucleotide sequence encoding a chimeric CD3G polypeptide, wherein the chimeric CD3G polypeptide comprises a human CD3G signal peptide, and at least 50 contiguous amino acid residues that are identical to the corresponding contiguous amino acid sequence of a human CD3G wherein the animal expresses the chimeric CD3G polypeptide.

51. The animal of claim 50, wherein the chimeric CD3G polypeptide has at least 50, at least 60, at least 70, at least 80, at least 90, at least 100, at least 110, at least 120, at least 130, at least 140, at least 150, at least 160, at least 170, or at least 180 contiguous amino acid residues that are identical to the corresponding contiguous amino acid sequence of a human CD3G.

52. The animal of claim 50 or 51, wherein the chimeric CD3G polypeptide comprises a sequence that is at least 90%, 95%, 96%, 97%, 98%, 99%, or 100% identical to SEQ ID NO: 6.

53. The animal of any one of claims 50-52, wherein the nucleotide sequence is integrated to an endogenous CD3G gene locus of the animal.

54. The animal of anyone of claims 33-53, wherein the animal further comprises a sequence encoding an additional human or chimeric protein.

55. The animal of claim 54, wherein the additional human or chimeric protein is CD3E, CD3D, programmed cell death protein 1 (PD-1), Programmed Cell Death 1 Ligand 1 (PD-L1), cytotoxic T-lymphocyte-associated protein 4 (CTLA-4), Lymphocyte Activating 3 (LAG-3), B And T Lymphocyte Associated (BTLA), tumor necrosis factor receptor superfamily member 9 (4-1BB), T-Cell Immunoreceptor With Ig And ITIM Domains (TIGIT), B7 Homolog 3 (B7-H3), T-cell Immunoglobulin and Mucin-Domain Containing-3 (TIM-3), Glucocorticoid-Induced TNFR-Related Protein (GITR), TNF Receptor Superfamily Member 4 (OX40), CD27, CD28, CD38, CD47, CD154, CD40, CD278, or CLDN18.2.

56. A method for making a genetically-modified, non-human animal, comprising:
replacing in at least one cell of the animal, at an endogenous CD3G gene locus, a sequence encoding a region of an endogenous CD3G with a sequence encoding a corresponding region of human CD3G.

57. The method of claim 56, wherein the sequence encoding the corresponding region of human CD3G comprises exon 1, exon 2, exon 3, exon 4, exon 5, exon 6, and/or exon 7, or a part thereof, of a human CD3G gene.

58. The method of claim 56 or 57, wherein the sequence encoding the corresponding region of human CD3G encodes SEQ ID NO: 6.

59. The method of any one of claims 56-58, wherein the animal is a mouse, and the endogenous CD3G locus comprises exon 1, exon 2, exon 3, exon 4, exon 5, exon 6, and/or exon 7, or a part thereof, of the mouse CD3G gene.

60. A method of making a genetically-modified non-human animal (e.g., mouse) cell that expresses a human or chimeric CD3G the method comprising:
replacing at an endogenous CD3G gene locus, a nucleotide sequence encoding a region of mouse CD3G with a nucleotide sequence encoding a corresponding region of human CD3G thereby generating a genetically-modified non-human animal cell that includes a nucleotide sequence that encodes the human or chimeric CD3G wherein the non-human animal cell expresses the human or chimeric CD3G.

61. The method of claim 60, wherein the nucleotide sequence encoding the human or chimeric CD3G is operably linked to a human CD3G regulatory region, e.g., promoter.

62. The method of claim 60, wherein the nucleotide sequence encoding the human or chimeric CD3G is operably linked to an endogenous CD3G regulatory region, e.g., promoter.

63. The method of anyone of claims 56-62, wherein the animal further comprises a sequence encoding an additional human or chimeric protein.

64. The method of claim 63, wherein the additional human or chimeric protein is CD3E, CD3D, PD-1, PD-L1, CTLA-4, LAG-3, BTLA, 4-1BB, TIGIT, B7-H3, TIM-3, GITR, OX40, CD27, CD28, CD38, CD47, CD154, CD40, CD278, and/or CLDN18.2.

65. A genetically-modified, non-human animal whose genome comprises at least one chromosome comprising:
(a) a sequence encoding a human or chimeric CD3E;
(b) a sequence encoding a human or chimeric CD3D; and/or
(c) a sequence encoding a human or chimeric CD3G.

66. The animal of claim 65, wherein the sequence encodes a human CD3E, a human CD3D, and a human CD3G.

67. The animal of claim 66, wherein the human CD3E comprises an amino acid sequence that is at least 70%, 75%, 80%, 85%, 90%, 95%, 96%, 97%, 98%, 99%, or 100% identical to SEQ ID NO: 2; the human CD3D comprises an amino acid sequence that is at least 70%, 75%, 80%, 85%, 90%, 95%, 96%, 97%, 98%, 99%, or 100% identical to SEQ ID NO: 4; and the human CD3G comprises an amino acid sequence that is at least 70%, 75%, 80%, 85%, 90%, 95%, 96%, 97%, 98%, 99%, or 100% identical to SEQ ID NO: 6.

68. The animal of claim 65, wherein the sequence encodes a chimeric CD3E, a human CD3D, and a human CD3G.

69. The animal of claim 68, wherein the chimeric CD3E comprises an amino acid sequence that is at least 70%, 75%, 80%, 85%, 90%, 95%, 96%, 97%, 98%, 99%, or 100% identical to SEQ ID NO: 66; the human CD3D comprises an amino acid sequence that is at least 70%, 75%, 80%, 85%, 90%, 95%, 96%, 97%, 98%, 99%, or 100% identical to SEQ ID NO: 4; and the human CD3G comprises an amino acid sequence that is at least 70%, 75%, 80%, 85%, 90%, 95%, 96%, 97%, 98%, 99%, or 100% identical to SEQ ID NO: 6.

70. A method of making a genetically-modified non-human animal comprising:
(a) replacing in at least one cell of the animal, at an endogenous CD3E gene locus, a sequence encoding a region of an endogenous CD3E with a sequence encoding a corresponding region of human CD3E;
(b) replacing in at least one cell of the animal, at an endogenous CD3D gene locus, a sequence encoding a region of an endogenous CD3D with a sequence encoding a corresponding region of human CD3D; and/or
(c) replacing in at least one cell of the animal, at an endogenous CD3G gene locus, a sequence encoding a region of an endogenous CD3G with a sequence encoding a corresponding region of human CD3G.

71. The method of claim 70, wherein step (b) and step (c) are performed at substantially the same time.

72. The method of claim 70 or 71, wherein step (a) is performed before or after step (b) and/or step (c).

73. A method of determining effectiveness of an anti-CD3E antibody, an anti-CD3D antibody, or an anti-CD3G antibody for the treatment of cancer, comprising:
administering the anti-CD3E antibody, the anti-CD3D antibody, or the anti-CD3G antibody to the animal of any one of claims 1-23, 33-55, and 65-69, wherein the animal has a tumor; and
determining the inhibitory effects of the anti-CD3E antibody, the anti-CD3D antibody, or the anti-CD3G antibody to the tumor.

74. The method of claim 73, wherein the tumor comprises one or more cancer cells that are injected into the animal.

75. The method of claim 73 or 74, wherein determining the inhibitory effects of the anti-CD3E antibody, the anti-CD3D antibody, or the anti-CD3G antibody to the tumor involves measuring the tumor volume in the animal.

76. The method of any one of claims 73-75, wherein the tumor cells are melanoma cells, pancreatic carcinoma cells, mesothelioma cells, or solid tumor cells.

77. A method of determining effectiveness of an anti-CD3E antibody, an anti-CD3D antibody, or an anti-CD3G antibody in inhibiting an immune response, comprising:
administering the anti-CD3E antibody, the anti-CD3D antibody, or the anti-CD3G antibody to the animal of any one of claims 1-23, 33-55, and 65-69, wherein the animal has a tumor; and
determining the effects of the anti-CD3E antibody, the anti-CD3D antibody, or the anti-CD3G antibody on the tumor.

78. The method of claim 77, wherein the tumor comprises one or more cancer cells that are injected into the animal.

79. The method of claim 77 or 78, wherein determining the effects of the anti-CD3E antibody, the anti-CD3D, or the anti-CD3G antibody on the tumor involves measuring the tumor volume in the animal.

80. A method of determining effectiveness of an anti-CD3E antibody, an anti-CD3D antibody, or an anti-CD3G antibody in inhibiting an immune response, comprising:
administering the anti-CD3E antibody, the anti-CD3D antibody, or the anti-CD3G antibody to the animal of any one of claims 1-23, 33-55, and 65-69, wherein the animal has a tumor; and
determining the percentage of T cells in peripheral blood and/or tumor tissue.

81. A method of determining effectiveness of a bispecific antibody targeting CD3 and a tumor associated antigen (TAA) for the treatment of cancer, comprising:
injecting cancer cells expressing the TAA to the animal of any one of claims 1-23, 33-55, and 65-69;
administering the bispecific antibody to the animal; and
determining the inhibitory effects of the bispecific antibody to the tumor.

82. The method of claim 81, wherein determining the inhibitory effects of the bispecific antibody to the tumor involves measuring the tumor volume in the animal.

83. The method of claim 81 or 82, wherein the TAA is BCMA.

84. A protein comprising an amino acid sequence, wherein the amino acid sequence is one of the following:
(a) an amino acid sequence set forth in SEQ ID NO: 2, 4, 6, or 66;
(b) an amino acid sequence that is at least 90% identical to SEQ ID NO: 2, 4, 6, or 66;
(c) an amino acid sequence that is at least 91%, 92%, 93%, 94%, 95%, 96%, 97%, 98%, or 99% identical to SEQ ID NO: 2, 4, 6, or 66;
(d) an amino acid sequence that is different from the amino acid sequence set forth in SEQ ID NO: 2, 4, 6, or 66 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2, 4, 6, or 66.

85. A nucleic acid comprising a nucleotide sequence, wherein the nucleotide sequence is one of the following:
(a) a sequence that encodes the protein of claim 84;
(b) SEQ ID NO: 7, 8, 9, 10, 11, 12, 13, 14, 15, 16, 17, 18, 35, 36, 63, 64, or 65;
(c) a sequence that is at least 90% identical to SEQ ID NO: 7, 8, 9, 10, 11, 12, 13, 14, 15, 16, 17, 18, 35, 36, 63, 64, or 65; and
(d) a sequence that is at least 91%, 92%, 93%, 94%, 95%, 96%, 97%, 98%, or 99% identical to SEQ ID NO: 7, 8, 9, 10, 11, 12, 13, 14, 15, 16, 17, 18, 35, 36, 63, 64, or 65.

86. A cell comprising the protein of claim 84 and/or the nucleic acid of claim 85.

87. An animal comprising the protein of claim 84 and/or the nucleic acid of claim 85.
